(19) United States
(12) Patent Application Publication
(10) Pub. No.: US 2026/0193671 A1
(43) Pub. Date: Jul. 9, 2026

(54) RECOMBINANT RSV VACCINE: METHODS OF MAKING AND USING THE SAME

(71) Applicant: BLUE LAKE BIOTECHNOLOGY, INC., Athens, GA (US)

(72) Inventors: Biao HE, Athens, GA (US); Hong JIN, Athens, GA (US); Maria Cristina GINGERICH, Athens, GA (US)

(73) Assignee: BLUE LAKE BIOTECHNOLOGY, INC., Athens, GA (US)

(21) Appl. No.: 19/125,921

(22) PCT Filed: Nov. 3, 2023
(86) PCT No.: PCT/US2023/078661

§ 371 Date: Apr. 30, 2025

Related U.S. Application Data
(60) Provisional application No. 63/382,453, filed on Nov. 4, 2022.

(51) Int. Cl. C12N 15/86; A61K 39/00; A61K 39/155; A61P 37/04; C07K 14/005

(52) U.S. Cl. CPC C12N 15/86; A61K 39/155; A61P 37/04; C07K 14/005; A61K 2039/5256; A61K 2039/543; A61K 2039/545; C12N 2760/18043; C12N 2760/18522; C12N 2760/18534

(57) ABSTRACT

Compositions and methods of inducing an immune response in a subject having RSV comprising administering a pharmaceutical composition comprising a prophylactic vaccine against RSV infection, wherein the vaccine comprises a live recombinant canine parainfluenza (CPI) vector backbone engineered to express a RSV F protein.

### REFERENCE TO RELATED APPLICATIONS

This application claims benefit of and priority to U.S. Provisional Application No. 63/382,453 filed on Nov. 4, 2022, which is incorporated by reference in its entirety.

### REFERENCE TO A SEQUENCE LISTING

The application contains a Sequence Listing which has been submitted electronically in .XML format and is hereby incorporated by reference in its entirety. Said .XML copy, created on Nov. 2, 2023, is named “065095.004PCT.xml” and is 42,181 bytes in size. The sequence listing contained in this .XML file is part of the specification and is hereby incorporated by reference herein in its entirety.

### FIELD

This invention relates generally to field of immunomodulation, and more particularly to compositions and methods for modulating immune responses against diseases from a respiratory syncytial virus (RSV) infection.

### BACKGROUND

Human RSV is the leading viral cause of lower respiratory illness and hospitalization in young children. The vast majority of children infected with RSV suffer from a mild upper respiratory tract infection; however, a small subset experience severe RSV-induced lower respiratory infection (LRI) and bronchiolitis that often requires hospitalization and can be life-threatening (Collins et al., Respiratory syncytial virus, In: Fields Virology. Knipe and Howley. eds., Lippincott Williams & Wilcins, New York (1996), pp. 1313-1351). Since nearly every child eventually is infected with RSV, and significant LRI develops in 20-30% of RSV-infected children, RSV causes more than 130,000 pediatric hospitalizations annually in the United States (Shay et al., JAMA, 282(5): 1440-1446 (1999), and World Health Organization, Initiative for Vaccine Research (IVR), Respiratory syncytial virus (RSV).

Some risk factors for the development of severe RSV-induced illness have been clearly identified, including premature birth (Navas et al., J. Pediatr., 121(3): 348-54 (1992)), bronchopulmonary dysplasia (Groothuis et al., Pediatrics, 82(2): 199-203 (1988)), congenital heart disease (MacDonald et al., N. Engl. J. Med, 307(1): 397-400 (1982)), and T cell immune deficiency (Mcintosh et al., J. Pediatr., 82(4): 578-90 (1973)). However, more than half of the children hospitalized with severe RSV-induced illness do not have an identified risk factor (Boyce et al., J. Pediatr., 137(6): 865-70 (2000)), which means that approximately 1-2% of otherwise healthy children without any identifiable risk factors suffer the potentially life-threatening consequences of RSV-induced illness (Collins et al., supra).

RSV-induced severe illness in children also has been correlated with the development of asthma (see, e.g., Sigurs et al., Pediatrics, 95(4): 500-505 (1995); Welliver et al., Pediatr. Pulmonol, 15(1): 19-27 (1993): Cifiientes et al., Pediatr. Pulmonol, 36(4): 316-321 (2003); Schauer et al., Eur. Respir. J., 20(5): 1277-1283 (2002); Sigurs et al., Am. J. Respir. Crit. Care Med., 161(5): 1501-1507 (2000); and Stein et al., Lancet, 354(9178): 541-545 (1999)). The basis for this association is unknown, but may be due to underlying genetic factors, immune dysfunction, antigen-specific responses, or structural lesions caused by lung remodeling after severe RSV disease.

Although RSV infection is almost universal by age three, reinfection occurs throughout life because natural RSV infection does not provide complete immunity (Hall et al., J. Infect. Dis., 163(4): 693-698 (1991), and Muelenaer et al., J. Infect. Dis., 164: 15-21 (1991)). In the elderly, RSV is an important cause of morbidity and mortality. In a retrospective cohort study, RSV was responsible for an annual average of IS hospitalizations and 17 deaths per 1.000 nursing home residents, whereas influenza accounted for an average of 28 hospitalizations and 15 deaths in the same setting (Garofalo et al., Pediatr. Allergy Immunol., 5(2): 111-117 (1994). Thus, RSV was isolated as frequently as influenza A in this population and was associated with comparable mortality as influenza A (Ellis et al., J. Am. Geriatr. Soc, 51(6): 761-72003; and Falsey et al., J. Infect. Dis., 772(2): 389-394 (1995)).

Currently there are no FDA-approved vaccines for the prevention of RSV infection or treatment of RSV-induced disease in children. The only FDA-approved medication for prophylaxis of RSV infection is SYNAGIS® (palivizumab) (MedImmune, Gaithersburg, MD), which is a humanized monoclonal antibody directed to an epitope in the A antigenic site of the RSV F protein administered to high-risk infants. Although SYNAGIS® represents a significant advance in the prevention of lower respiratory tract acute RSV disease and mitigation of lower respiratory tract infection, it has not been shown to be effective against RSV infection in the upper respiratory tract at permissible doses. In 2023, FDA approved half-life extended long-acting RSV monoclonal antibody Beyfortus (nirsevimab), which was recommended for all infants aged <8 months who are born during or entering their first RSV season and for infants and children aged 8-19 months who are at increased risk for severe RSV disease and are entering their second RSV season.

RSV vaccine development has suffered from a legacy of vaccine-enhanced disease in children after natural RSV infection (Kim et al., Am. J. Epidemiol, 89(4): 422-434 (1969); and Kapikian et al., Am. J. Epidemiol, 89(4): 405-421 (1969)). For example, in the early 1960s a formalin-inactivated alum-precipitated vaccine candidate (FI-RSV) was administered to RSV-naïve infants in the early 1960s, and although immunogenic, it did not protect the children against natural infection. In addition, vaccinees subsequently infected with RSV had increased hospitalization rates and more severe illness, including two deaths, relative to control children immunized with formalin-inactivated parainfluenza virus (Kapikian et al., supra, Chin et al., Am. J. Epidemiol., 89(4): 449-463 (1969); and Polack et al., J. Exp. Med, 196(6): 859-65 (2002)). Other approaches to RSV immunization have included live attenuated RSV, RSV subunit proteins, and parainfluenza virus chimeras. Live attenuated RSV vaccines have been tested in clinical trials of RSV-naïve infants, but have not been shown to achieve genetic stability of mutations, the optimal balance between attenuation for safety in infants, or a protective immune response (Karron et al., J. Infect. Dis., 191(7): 1093-1104 (2005); and Bukreyev et al., J. Virol. 79(15): 9515-9526 (2005)). Protein subunit vaccines based on RSV G and F proteins have been safely administered to adults and RSV-seropositive children, but are modestly immunogenic (Tristram et al., Vaccine, 12(6): 551-556 (1994)).

Therefore, there remains a need for compositions and methods to effectively and safely prevent or treat RSV infection.

Thus, the invention provides such compositions and methods for effectively and safely preventing or treating RSV infection in a mammal, preferably a human.

### SUMMARY

In accordance with the purpose(s) of this invention, as embodied and broadly described herein, this invention, in one aspect, relates to a viral expression vector comprising a parainfluenza virus 5 (PIV5) genome having a heterologous nucleic acid sequence with at least 95% sequence identity to SEQ ID NO: 1, wherein the viral expression vector expresses a heterologous polypeptide comprising a live recombinant canine parainfluenza (CPI) vector backbone engineered to express a RSV F protein as target antigen. In one embodiment, the RSV F protein is encoded by a wildtype or mutated RSV F protein gene. In another embodiment, the RSV F protein gene is codon optimized for expression in a human subject. In yet another embodiment, the RSV F protein gene is inserted between a SH and HN junction of a CPI antigenomic cDNA, wherein the CPI antigenomic cDNA is sequenced with primers having a nucleic acid sequence with at least 95% sequence identity to SEQ ID NO: 2, 3, 4, 5, 6, 7, 8, 9, 10, 11, 12, 13, 14, 15, 16, 17, 19, or 19. In another embodiment, the parainfluenza (CPI) vector backbone engineered to express a RSV F protein comprises a 22 amino acid extension as part of its cytoplasmic tail.

In another aspect, the invention relates to a pharmaceutical composition comprising a parainfluenza virus 5 (PIV5) viral expression vector having a heterologous nucleic acid sequence with at least 95% sequence identity to SEQ ID NO: 1, wherein the viral expression vector expresses a heterologous polypeptide comprising a live recombinant canine parainfluenza (CPI) vector backbone engineered to express a RSV F protein as a target antigen. In one embodiment, the RSV F protein is encoded by a wildtype or mutated RSV F protein gene, wherein the RSV F protein gene is codon optimized for expression in a human subject. In another embodiment, the RSV F protein gene is inserted between a SH and HN junction of a CPI antigenomic cDNA, wherein the CPI antigenomic cDNA is sequenced with primers having a nucleic acid sequence with at least 95% sequence identity to SEQ ID NO: 2, 3, 4, 5, 6, 7, 8, 9, 10, 11, 12, 13, 14, 15, 16, 17, 19, or 19. In yet another embodiment, the parainfluenza (CPI) vector backbone engineered to express the RSV F protein comprises a 22 amino acid extension as part of its cytoplasmic tail. In one other embodiment, the live recombinant canine parainfluenza (CPI) vector backbone engineered to express a RSV F protein is a prophylactic vaccine against RSV infection.

In yet another aspect, the invention relates to a method of inducing an immune response in a subject having RSV comprising administering a prophylactic vaccine against RSV infection, wherein the vaccine comprises a live recombinant canine parainfluenza (CPI) vector backbone engineered to express a RSV F protein as described above. In another embodiment, the vaccine induces RSV F-protein specific serum antibodies and cell mediated responses. In another embodiment, the RSV-F specific serum antibodies and cell mediated responses are associated with a reduced incidence of an RSV-induced pathological lung response compared to an immune response obtained by administering a formalin-inactivated RSV (FI-RSV). In one other embodiment, the pathological lung response is selected from a group consisting of: peribronchiolitis, perivasculitis, interstitial pneumonia, and alveolitis. In another embodiment, the vaccine is administered intranasally, intramuscularly, topically, or orally. In another embodiment, the vaccine is administered in a single or multiple dose regimen.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIGS. 1A-1B show RSV F-specific immune responses in immunized AGMs. RSV F-specific cellular immune response in immunized African green monkeys (AGMs). PBMCs were isolated from immunized AGMs one day prior to vaccination, and at 14 and 28 days post-immunization. The PBMCs were stimulated with an RSV F peptide pool, and different cytokine levels in CD4⁺ (FIG. 1A) and CD8⁺ (FIG. 1B) cells were quantified by ICS and expressed at % sum of all cytokines. Group 1=control. Group 6=CPI-RSV-F.

FIGS. 2A-2B show a plasmid map of pSP28 (FIG. 2A) and pAB94 (FIG. 2B).

FIG. 3 shows the CPI-RSV-F vaccine vector plasmid and map.

FIG. 4 shows an illustrative overview of virus rescue.

FIGS. 5A-5C show representative images of RSV F-protein percent expression assay CPI-RSV-F pre-MVS.

FIGS. 6A-6C show representative images of RSV F-protein percent expression assay CPI-RSV-F MVS.

FIG. 7 shows an illustration of the CHD03 experimental tinmeline.

FIG. 8 shows an illustration of the CHD04 experimental timeline.

FIGS. 9A-9B show CHD03 (FIG. 9A) and CHD04 (FIG. 9B) RSV-F serum antibody titers. Naïve BALB/c mice were treated with PBS or immunized intranasally with 10⁵ PFU of PIV5-RSV-F, PIV5ΔSH-RSV-F, CPI-RSV-F, CPIΔSH-RSV-F, or RSV_rA2. Sera were collected at 4 weeks post-immunization. RSV-F-specific IgG antibody endpoint titers were determined by ELISA (N=5). Horizontal lines represent the geometric mean antibody titer of each group. Statistical significance was determined by ANOVA and Dunnett's multiple comparison tests. ****P<0.0001, significance between PBS and vaccine groups. LOD, limit of detection.

FIGS. 10A-10B show CHD03 and CHD04 RSV-F ELISPOT titers. Naïve BALB/c mice were treated with PBS or immunized intranasally with 10⁵ PFU of PIV5-RSV-F, PIV5ΔSH-RSV-F, CPI-RSV-F, CPIΔSH-RSV-F, or RSV_rA2. Splenocytes were harvested and stimulated with an RSV F peptide. Results are presented as number of IFN-7-secreting cells per 10⁶ splenocytes (N=5). Horizontal lines in represent the geometric mean ELISPOT titer. Statistical significance was determined by ANOVA and Dunnett's multiple comparison tests. *P<0.05, **P<0.01. ***P<0.001, significance between PBS and vaccine groups.

FIGS. 11A-11B show CHD03 (FIG. 11A) and CHD04 (FIG. 11B) lung titers.

FIG. 12 shows a lung RSV challenge virus titer following RSV challenge.

FIG. 13 shows a nasal RSV challenge virus titer following RSV challenge in nasal wash.

FIG. 14 shows RSV neutralizing antibody responses.

FIG. 15 shows anti-RSV F protein IgG antibody responses by ELISA.

FIGS. 16A-16D are bar graphs showing the expression of RSV NS1, IL-4, IL-2, and IFN-γ mRNA.

FIGS. 17A-17F are bar graphs showing serum RSV-specific antibody (Ab) titers before and after vaccination with BLB201 (CPI-RSV-F) for younger adults (Group 1) and older adults (Group 2). FIGS. 17A-17B show RSV neutralizing Abs by microneutralization assay; FIGS. 17C-17D show F-specific RSV IgA Abs and FIGS. 17E-17F show F-specific IgG Abs by ELISA. Individual log 2 titers, and mean log 2 titers with the respective standard deviations (error bars) at Days 1, 15 and 29 by age group are shown in the graphs in the left column, and geometric mean fold rises from baseline (GMFRs) on Day 15 and Day 29 by age group are shown in the graphs in the right column. *P<0.05; **P<0.01; ***P<0.001 by Wilcoxon test.

FIGS. 18A-18D are line graphs showing individual subject's serum RSV neutralizing (nAb) titers of Group 1 (FIG. 18A) and Group 2 (FIG. 18B) and nasal F-specific IgA antibody (Ab) titers before and after vaccination with BLB201 (CPI-RSV-F) (at Days 1, 15 and 29) (FIG. 18C-18D) and by age group 1 and 2.

FIGS. 19A-19D are bar graphs showing serum PIV5 antibody (Ab) titers before and after vaccination with BLB201 (CPI-RSV-F) for two vaccination groups. FIGS. 19A-19B show PIV5 neutralizing Abs by microneutralization assay; and FIGS. 19C-19D show PIV5-specific RSV IgG Abs by ELISA. Individual log 2 titers, and mean log 2 titers with the respective standard deviations (error bars) at Days 1, 15 and 29 by age group are shown in the graphs in the left column, and geometric mean fold rises from baseline (GMFRs) on Day 15 and Day 29 by age group are shown in the graphs in the right column. *P<0.05; **P<0.01; ***P<0.001 by Wilcoxon test.

FIGS. 20A-20C are bar graphs showing the relationships between response rates to PIV5-RSV vaccination based on PIV5 neutralizing antibody (nAb) levels at baseline (pre-vaccination). Percentage of subjects with (FIG. 20A) PIV5 neutralizing (nAb) seroresponses to the PIV5-RSV vaccine (≥1.5-fold rise in titer) and (FIG. 20B) RSV neutralizing (nAb) seroresponses to the PIV5-RSV vaccine (≥1.5-fold rise in titer) by subgroups based on individual subject's baseline PIV5 nAb titer being below (L) or above (H) the baseline geometric mean titer for PIV5 nAbs for each age group (Group 1 and Group 2). FIG. 20C shows percentage of subjects with nasal F-specific IgA responses to the PIV5-RSV vaccine (≥2-fold rise in titer) by subgroups based on individual baseline nasal F-specific IgA titer being below (L) or above (H) the baseline geometric mean titer for nasal F-specific IgA for each age group (Group 1 and Group 2).

FIGS. 21A-21B are bar graphs showing nasal IgA antibody titers before and after vaccination for Groups 1 and 2. F-specific RSV IgA Abs by ELISA. Individual log 2 titers, and mean log₂ titers with the respective standard deviations (error bars) at Days 1, 15 and 29 by age group are shown in the left graph (FIG. 21A), and geometric mean fold rises from baseline (GMFRs) on Day 15 and Day 29 by age group are shown in the graph (FIG. 21B).

FIGS. 22A-22J show RSV F-specific CD4⁺ and CD8⁺ T cell responses before and after vaccination of BLB201 (CPI-RSV-F). Percentages of CD4⁺ T cells (upper graphs) and CD8⁺ T cells (22A-D) by age group on Days 1, 15 and 29 for each individual (represented by symbols and contiguous line) by expression of the indicated immune marker. FIGS. 22E & 22F show mean percentages of CD4⁺ T cells (left graphs) and CD8⁺ T cells (right graphs) by age group on Days 1, 15 and 29 by expression of the indicated Th1/cytotoxic marker combinations (IFN-γ, TNF-α. CD107a) represented in stacked histograms. FIGS. 22G & 22H show geometric mean fold rises from baseline (GMFRs) on Day 15 and Day 29 by age group for CD4⁺ and CD8⁺ T cells. FIGS. 22G and 22H show fold change of CD4 and CD8 T cell responses at Days 15 and 29 postvaccination. FIGS. 22I & 22J are pie-chart representations of the proportions of F-specific CD4⁺ and CD8⁺ T cells expressing 1, 2 or 3 Th1/cytotoxic markers (IFN-γ. TNF-α, CD107a) at Days 15 and 29 in Groups 1 and 2. * P<0.05; **P<0.01; ***P<0.001, ns: not significant.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The words “preferred” and “preferably” refer to embodiments of the invention that may afford certain benefits, under certain cirmumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

As described herein, the term “vaccinating” designates typically the sequential administration of one or more antigens to a subject, to produce and/or enhance an immune response against the antigen(s). The sequential administration includes a priming immunization followed by one or several boosting immunizations.

Within the context of the present invention, the term “pathogen” refers to any agent that can cause a pathological condition. Examples of “pathogens” include, without limitation, cells (e.g., bacteria cells, diseased mammal cells, cancer mammal cells), fungus, parasites, viruses, prions or toxins. Preferred pathogens are infectious pathogens. In a particular embodiment, the infectious pathogen is a virus, such as the coronaviruses.

An antigen, as used therein, designates any molecule which can cause a T-cell or B-cell immune response in a subject. An antigen specific for a pathogen is, typically, an element obtained or derived from said pathogen, which contains an epitope, and which can cause an immune response against the pathogen. Depending on the pathogenic agent, the antigen may be of various nature, such as a (poly)peptide, protein, nucleic acid, lipid, cell, etc. Live weakened forms of pathogens (e.g., bacteria, viruses), or killed or inactivated forms thereof may be used as well, or purified material therefrom such as proteins, peptides, lipids, etc. The antigen may be naturally-occurring or artificially created. It may be exogenous to the treated mammal, or endogenous (e.g., tumor antigens). The antigen may be produced by techniques known per se in the art, such as for instance synthetic or recombinant technologies, or enzymatic approaches.

In a particular embodiment, the antigen is a protein, polypeptide and/or peptide. The terms “polypeptide”, “peptide” and “protein” are used interchangeably herein to refer to a polymer of amino acid residues. The terms also apply to amino acid polymers in which one or more amino acid residues may be modified or non-naturally occurring residues, such as an artificial chemical mimetic of a corresponding naturally occurring amino acid. It should be understood that the term “protein” also includes fragments or variants of different antigens, such as epitope-containing fragments, or proteins obtained from a pathogen and subsequently enzymatically, chemically, mechanically or thermally modified.

A “therapeutically effective amount” means the amount of a compound (e.g., a PIV5-based composition as described herein) that, when administered to a subject for treating a state, disorder or condition, is sufficient to effect such treatment. The “therapeutically effective amount” will vary depending on the compound or bacteria administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “pharmaceutically acceptable composition” as used herein refers to a composition comprising at least one compound as disclosed herein formulated together with one or more pharmaceutically acceptable carriers.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “dose” means a single amount of a compound or an agent that is being administered thereto; and/or “regimen: which means a plurality of pre-determined doses that can be different in amounts or similar, given at various time intervals, which can be different or similar in terms of duration. In some embodiments, a regimen also encompasses a time of a delivery period (e.g., agent administration period, or treatment period). Alternatively, a regimen is a plurality of predetermined plurality pre-determined vaporized amounts given at pre-determined time intervals.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

Within the context of the present invention, the term “pathogen” refers to any agent that can cause a pathological condition. Examples of “pathogens” include, without limitation, cells (e.g., bacteria cells, diseased mammal cells, cancer mammal cells), fungus, parasites, viruses, prions or toxins. Preferred pathogens are infectious pathogens. In a particular embodiment, the infectious pathogen is a virus, such as the coronaviruses.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As used herein, the term “parainfluenza virus 5” (PIV5) includes, for example and not limitation, strains KNU-11, CC-14, D277, 1168-1, and 08-1990. Non-limiting examples of PIV5 genomes are listed in GenBank Accession Nos. NC_006430.1, AF052755.1, KC852177.1, KP893891.1, KC237065.1. KC237064.1 and KC237063.1, which are hereby incorporated by reference.

As used herein, the term “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA. In the context of the present invention, the term also encompasses the yield of RSV F gene mRNA and RSV F proteins achieved following expression thereof.

As used herein, the term “F protein” or “Fusion protein” or “F protein polypeptide” or “Fusion protein polypeptide” refers to a polypeptide or protein having all or part of an amino acid sequence of an RSV Fusion protein polypeptide. Numerous RSV Fusion and Attachment proteins have been described and are known to those of skill in the art. WO/2008/114149, which is herein incorporated by reference in its entirety, sets out exemplary F and G protein variants (for example, naturally occurring variants).

The materials described as making up the various elements of the disclosure are intended to be illustrative and not restrictive. Many suitable materials that would perform the same or a similar function as the materials described herein are intended to be embraced within the scope of the disclosure. Such other materials not described herein can include, but are not limited to, for example, materials that are developed after the time of the development of the disclosure.

### II. CPI-RSV-F Compositions

Respiratory syncytial virus (RSV) is a member of the genus Pneumovirus of the family Paramyxoviridae. Human RSV (HRSV) is the leading cause of severe lower respiratory tract disease in young children and is responsible for considerable morbidity and mortality in humans. RSV is also recognized as an important agent of disease in immunocompromised adults and in the elderly. Due to incomplete protection to RSV in the infected host after a natural infection. RSV may infect multiple times during childhood and adult life.

This virus has a genome comprised of a single strand negative-sense RNA, which is tightly associated with viral protein to form the nucleocapsid. The viral envelope is composed of a plasma membrane derived lipid bilayer that contains virally encoded structural proteins. A viral polymerase is packaged with the virion and transcribes genomic RNA into mRNA. The RSV genome encodes three transmembrane structural proteins, F, G, and SH, two matrix proteins, M and M2, three nucleocapsid proteins N, P, and L, and two nonstructural proteins, NS1 and NS2.

Fusion of HRSV with infected cell membranes is thought to occur at the cell surface and is a necessary step for the transfer of viral ribonucleoprotein into the cell cytoplasm during the early stages of infection. This process is mediated by the fusion (F) protein, which also promotes fusion of the membrane of infected cells with that of adjacent cells to form a characteristic syncytia, which is both a prominent cytopathic effect and an additional mechanism of viral spread. Accordingly, neutralization of fusion activity is important to block virus infection and is important in host immunity. Indeed, monoclonal antibodies developed against the F protein have been shown to neutralize virus infectivity and inhibit membrane fusion (Calder et al., 2000, Virology 271: 122-131).

The F protein of RSV shares structural features and limited, but significant amino acid sequence identity with F glycoproteins of other paramyxoviruses. It is synthesized as an inactive precursor of 574 amino acids (F0) that is cotranslationally glycosylated on asparagines in the endoplasmic reticulum, where it assembles into homo-oligomers. Before reaching the cell surface, the F) precursor is cleaved by a protease into F2 from the N terminus and F1 from the C terminus. The F2 and F1 chains remain covalently linked by one or more disulfide bonds.

CPI-RSV-F is a parainfluenza virus (PIV5) based RSV vaccine expressing the RSV F protein, is provided herein as prophylactic intranasal vaccines to prevent RSV infection and serious complications associated with RSV infection. CPI-RSV-F was designed to induce immune responses to the F protein of RSV, which is the main antigenic protein that is highly conserved between the RSV subgroups A and B. Anti-F antibodies inhibit virus entry into host cells and RSV F is a proven vaccine target based on the efficacy data from the commercial product palivizumab (RSV F monoclonal antibody).

The disclosure provides CPI-RSV-F compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

### A. Pharmacology Summary of RSV Studies

The disclosure provides CPI-RSV-F compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

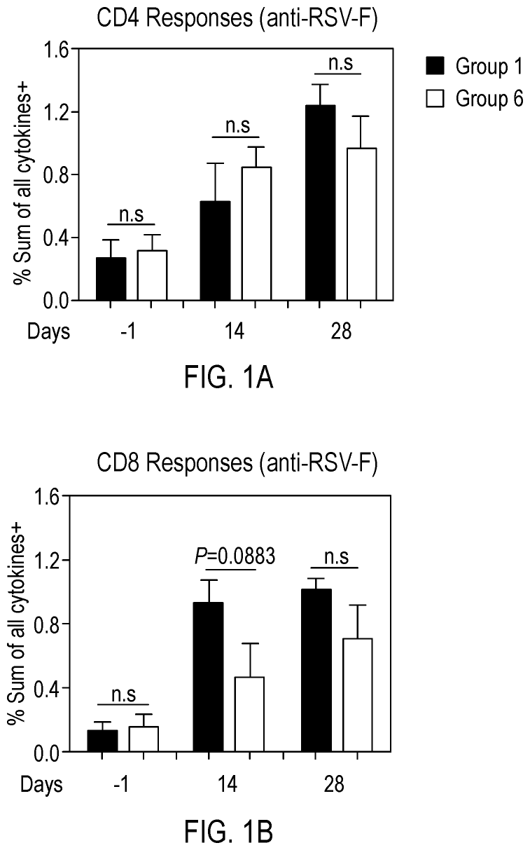

Previously published studies based on W3A strain of PIV5 construct engineered to express RSV-F protein included immunogenicity and challenge studies in mice, cotton rats and African green monkeys (1,2,3). As part of these studies, permissiveness for PIV5 replication was confirmed in cotton rats and African monkeys (1, 2). In addition, the following studies were performed: 1) compared vector constructs based on CPI vs. W3A PIV5 strain; 2) compared vaccine constructs expressing RSV pre-fusion versus wt F-protein in place of PIV5 SH gene (ΔSH) or inserted between PIV5 SH and NH (SH-NH) or between HN and L (HN-L). These studies led to the selection of CPI-RSV-F (CPI-RSV-F), containing the full length of RSV F-protein inserted between SH and HN gene (FIG. 1) for use in the initial human studies.

In addition, the protective efficacy of CPI-RSV-F vaccine has been evaluated in RSV challenge studies conducted in mice and cotton rats. Immunization with a single intranasal dose protected animals from RSV infection based on significantly reduced RSV viral titers observed in lung and nasal washes of immunized animals compared to controls.

More recent preclinical proof of concept studies conducted with the vaccine vector construct CPI-RSV-F included immunogenicity and challenge studies conducted by Blue Lake Biotechnology Inc. in mice and African green monkeys. In addition, preclinical data from a NIH sponsored study of the CPI-RSV-F construct in a cotton rat challenge study are summarized for which Blue Lake Biotechnology Inc. provided the vaccine product. The CPI-RSV-F vaccine used in these more recent non-clinical studies used a prior vaccine vector construct (rescued from BHK cells) that is the same as the vector construct used for clinical trial material (rescued from 293Nero cells), and similarly produced using serum-free Vero cells as the substrate and formulated with sucrose phosphate glutamate (SPG) buffer. These non-clinical studies included W3AΔSH-RSV-F (PIV5 W3A strain engineered to express RSV F protein) as an active comparator.

Phase 1 clinical trial of CIP-RSV-F demonstrated RSV vaccine safety in young adults (Group 1) and older adults (Group 2). The vaccine elicited serum RSV-specific and nasal IgA antibody responses as well as potent cellular immune Reponses (Spearman et al., Sci. Adv. 9, eadj7611 (2023)).

In summary, the non-clinical studies in various animal models have demonstrated the ability of CPI-RSV-F to induce RSV F specific immune responses, as observed by F-specific antibody and cell mediated responses following a single intranasal dose. CPI-RSV-F was well tolerated in these animal models and no indication of sensitization following vaccination with CPI-RSV-F was observed. Recent non-clinical studies conducted in mice, cotton rats and African green monkeys with the CPI-RSV-F vaccine construct (vaccine based on CPI PIV5 strain) are summarized below. These studies included an active comparator (vaccine vector based on PIV5 W3A strain) with RSV F protein replacing SH gene (ΔSH). See FIG. 1 for further detail on vaccine vector constructs. For an overview of non-clinical studies conducted with various vaccine vector constructs, see Table 1.

TABLE 1
Non-clinical study overview of prior vaccine constructs
studies and recent studies with CPI-RSV-F construct.

| Study | | | Vaccine | Reference |
|---|---|---|---|---|
| type | Species | Vaccine vector | route | report |
| Immunogenicity/challenge | Mice | W3A strain with wt RSV F | i.n. | Phan et al. |
| study | | protein inserted into PIV5 | | 2014 |
| | | HN-L region | | |
| Immunogenicity/challenge/safety | Mice and | W3A strain with wt RSV or | i.n. and | Phan et al |
| study | Cotton rats | prefusion RSV F protein | s.c. | 2017/ |
| | | inserted at deleted SH | | Sigmovir |
| | | region or between SH and | | Protocol |
| | | HN | | #XV-131 |
| | | | | Study report |
| Immunogenicity/challenge/safety | Cotton rats | W3A strain with RSV F | i.n | Wang et al |
| study | and | protein inserted into the | | 2017 |
| | African | intergenic regions of PIV5 | | |
| | Green | HN and L | | |
| | monkeys | | | |

Recent studies with control (construct to be used in clinical study and used in toxicology study)

| | | | | |
|---|---|---|---|---|
| Immunogenicity/challenge | Mice | CPI strain with RSV F | i.n. | Report 1 |
| study | | protein between SH and HN | | |
| | | or replacing HN (CPI-RSV- | | |
| | | F, CPIΔSH-RSV-F) or | | |
| | | controls W3A-RSV-F and | | |
| | | W3AΔSH-RSV-F | | |
| | | construct rescued from | | |
| | | BHK cells | | |
| Immunogenicity | African | CPI strain with RSV F | i.n. | No report. |
| study | green | protein between SH and NH | | |
| | Monkeys | (CPI-RSV-F) or control | | |
| | | W3AΔSH-RSV-F construct | | |
| | | rescued from BHK cells | | |
| Immunogenicity/challenge/safety | Cotton rats | CPI strain with RSV F | | Sigmovir |
| study | | protein between SH and NH | | Study XV- |
| | | (CPI-RSV-F, CPI-RSV-F) | | 238 report |
| | | or control | | (NIH |
| | | W3AΔSH-RSV-F | | sponsored) |
| | | Construct rescued from | | |
| | | BHK cells | | |

i. Studies in Mice, Cotton Rats, and Monkeys with Related W3A PIV5 Vector Expressing RSV F Protein

PIV5 (W3A)-RSV-F and RSV-G protein study in Balb/c mice (3, Phan et al. 2014): In this study Balb/c mice received a single intranasal dose of PIV5-RSV-F (10⁶ PFU dose in 50 μl) followed by RSV challenge. The PIV5 W3A vaccine vector construct in this study consisted of wild type RSV F protein inserted into the PIV5 HN and L intergenic regions. A single intranasal dose resulted in IgG2a/IgG1 RSV responses similar to that observed after wild-type RSV A2 infection at Day 21 after immunization.

PIV5 (W3A) expressing wild-type or Prefusion RSV F protein challenge study in mice and cotton rats (1, Phan et al 2017): This study evaluated PIV5 vectored vaccines that were improved by changing location of F-protein insertion (inserted at SH-HN junction of PIV5 or replacing the SH with the RSV F protein gene). In addition, this study evaluated both the wild type (wt) F-protein or a prefusion conformation F-protein (pF).

Mice were immunized intranasally with single dose of W3AΔSH-RSV-F (RSV F protein gene inserted at deleted SH region of PIV5) expression wild type F protein or prefusion stabilized RSV F mutant (W3AΔSH-RSV-pF) or improved vector W3A-RSV-F SH-HN or W3A-RSV-pF SH-HN (the F-protein gene inserted at the SH-HN junction) at 1×10⁶ PFU. The study groups were as follows (Table 2).

TABLE 2
Study groups in mice challenge study

| | Number of | |
|---|---|---|
| Vaccine/route | animals | Challenge virus |
| PBS | 10 | 1 × 10⁶ PFU RSV A/A2 |
| RSV A2 | 5 | 1 × 10⁶ PFU RSV A/A2 |
| W3A(HN-L)-RSV-F, | 10 | 1 × 10⁶ PFU RSV A/A2 |
| 10⁶ PFU, i.n. | | |
| W3A(SH-HN)-RSV- | 10 | 1 × 10⁶ PFU RSV A/A2 |
| F,, 10⁶ PFU, i.n. | | |
| W3A(SH-HN)-RSV- | 10 | 1 × 10⁶ PFU RSV A/A2 |
| pF, 10⁶ PFU, i.n. | | |
| W3AΔSH-RSV-F, | 10 | 1 × 10⁶ PFU RSV A/A2 |
| 10⁶ PFU, i.n. | | |
| W3AΔSH-RSV-pF, | 10 | 1 × 10⁶ PFU RSV A/A2 |
| 10⁶ PFU, i.n. | | |

Following immunization, both humoral and cell mediated immune responses were observed. The highest neutralizing antibody responses were detected with vaccine construct using the wt F protein. Also, vaccine constructs containing the wt F inserted prior to the HN junction with ΔSH seemed to be most immunogenic. The level of cell-mediated immune responses (based on IFN-gamma using ELISPOT) was similar between the various vaccine constructs.

Mice were challenged 28 days after immunization with RSV A2 to determine protective efficacy. Challenge virus was only obtained from one of five mice at day 4 post challenge in the W3AΔSH-RSV-F group with none of the mice in the other vaccination groups. In the PBS control group challenge virus was recovered from all mice.

A similar study was conducted in cotton rats which are more permissive to RSV infection. Rats were immunized with low dose of 10³ PFU of the modified vectors (W3A-RSV-F (SH-HN), W3A-RSV-pF, W3AΔSH-RSV-F) containing wild type or perfusion stabilized F-protein (pF) or 10² PFU of W3ΔASH-RSV-pF.

Immune responses were observed in all groups including neutralizing antibody responses against RSV A Tracy strain (97% identical to RSV A/A2 strain). Similar to the mouse study, the groups immunized with vaccine constructs containing the wt F protein had higher antibody levels compared to the pF groups, with F insertion prior to the HN gene junction with ΔSH having the highest values (approx. titer of 128). The neutralization antibody titers to RSV/B/18537 were significantly lower than to RSV/A Tracy strain, only the W3A(SH-HN)—RSV-F and W3AΔSH-RSV-F groups had significant antibody levels (approx. titer of 8) detected. After RSV challenge on Day 28 with 1.21×10⁵ PFU of RSV/A/Tracy, reduction in RSV viral load in nasal washes (1.4-1.66 Log₁₀ reduction) and lung lavage fluid (2-3 Log₁₀ reduction) was observed for all vaccine dose groups. The RSV challenge virus was recovered from all mice in the PBS control group (approx. 10⁵ PFU per nasal wash, or 10⁵ PFU/g lung wash).

Overall, these initial studies in the mouse and cotton rat models did not show evidence of the prefusion F-protein being more immunogenic and protective then the wild type F protein. Also, no difference was observed in the vector performance between ΔSH and the SH-HN insertion vector construct for the intranasal route.

Sigmovir Protocol #XV-131 Study report (Phan et al. 2017) challenge studies in cotton rat using intranasal and subcutaneous route: In a follow-on study (1. Phan et al. 2017) the efficacy, immunogenicity and safety of higher doses 10⁵ and 10⁶ PFU of W3A(SH-NH)-RSV-F and control were tested in a cotton rat challenge study evaluating two different routes intranasal (i.n.) and subcutaneous (s.c). This study also included a positive control for enhancement of disease (animals immunized with FI-RSV followed by RSV challenge and a positive control group consisting of animals that were pre-infected with RSV followed by RSV challenge. Animals in this study were challenged with RSV A2 on Day 49 followed by necropsy and histology 5 days later (Day 54). The study groups in this study were as follows (Table 3).

TABLE 3
Study groups in Protocol #XV-131.

| | N = number | |
|---|---|---|
| Vaccine/route | of animals | RSV/A2 Challenge |
| PBS i.m. | 5 | PBS |
| PBS i.m | 5 | 5Log₁₀ PFU |
| FI-RSV i.m | 5 | 5Log₁₀ PFU |
| RSV/A2 Live, i.n. (positive | 5 | 5Log₁₀ PFU |
| control) | | |
| W3A(SH-HN)-RSV-F, 10⁵ | 5 | 5Log₁₀ PFU |
| PFU i.n. | | |
| W3A(SH-HN)-RSV-F, 10⁵ | 5 | 5Log₁₀ PFU |
| PFU s.c | | |
| W3A(SH-HN)-RSV-F, 10⁶ | 5 | 5Log₁₀ PFU |
| PFU s.c | | |
| W3AΔSH-RSV-F, 10⁵ PFU | 5 | 5Log₁₀ PFU |
| i.n. | | |
| W3AΔSH-RSV-F, 10⁵ PFU | 5 | 5Log₁₀ PFU |
| s.c. | | |
| W3AΔSH-RSV-F, 10⁶ s.c. | 5 | 5Log₁₀ PFU |

Immunogenicity: W3A(SH-HN)—RSV-F resulted in similar neutralizing antibody titer in 10⁵ and 10⁶ dose between i.n and s.c group. W3AΔSH-RSV-F vaccination induced slightly higher neutralizing antibody titers in i.n group compared to s.c group, with 10⁶ dose resulting in a slightly higher titer, although it was not statistically significant.

Efficacy: W3A(SH-HN)—RSV-F resulted in complete protection in the lower respiratory tract by either i.n or s.c administration. Animals also had significantly lower viral loads in the upper respiratory tract.

W3A(SH-HN)-RSV-F vaccination resulted in complete protection in the lower respiratory tract by s.c administration and almost complete protection by i.n administration based on reduced titers (average titer of 10³ PFU) observed in lung and nasal washes compared to control animals sham immunized with PBS (average titer of 10⁵ PFU).

Safety: Lung sections from the different groups were examined for hallmarks of pulmonary inflammation: peribronchiolitis, perivasculitis, interstitial pneumonia, and alveolitis and scored for severity. The largest lesions were observed in the FI-RSV-immunized, RSV-challenged group (positive control group). Pulmonary changes were moderate in the groups immunized with W3A-RSV-F (SH-HN) or W3AΔSH-RSV-F (intranasally or subcutaneously), with levels below those observed in the RSV-inununized, RSV-challenged positive control group and similar to those in the PBS sham-immunized, RSV-challenged group.

Cytokine levels measured in lung tissues at 5 days post-challenge by quantitative real-time PCR (qPCR) were not indicative of potential of enhanced infection. The IL4 mRNA level was significantly elevated only in the FI-RSV-vaccinated group, consistent with the histopathology results and the enhanced disease phenotype. IFNγ-mRNA levels were the highest in the sham-immunized and FI-RSV-immunized groups. IFN-mRNA levels were similarly low between groups immunized with the PIV5-based candidates and the RSV-immunized group. IL-2 mRNA levels were similar in all groups, but the average IL-2 level in the FI-RSV immunized group was significantly higher than that in the other groups.

After intranasal challenge of immunized mice with RSV A2 (10⁶ PFU in 50 μl) at Day 28 after immunization, lung sections obtained at 4 days after challenge showed no exacerbation of lung lesions relative to RSV-A2 immunized mice. Protective immunity was observed as assessed by viral load in lung tissues (n=5 mice per group).

PIV5 (W3A) expressing RSV F or G protein challenge study in cotton rats and African green monkeys (2, Wang et al 2017): This study evaluated PIV5 vectored RSV F protein in cotton rats and African green monkeys for replication, immunogenicity and efficacy of protection against RSV challenge. In this study, the F protein was inserted into the intergenic regions of PIV5 HN and L gene.

PIV5 replication permissiveness in cotton rats and African green monkeys: Cotton rats (n=4 per group) were inoculated intranasally with 1×10⁵ PFU of PIV5 at volumes of 10 μl or 10 μl. Viral titers were observed in nose homogenates (up 1×10⁴ PFU) at day 4 which cleared mostly by day 6. The larger inoculum volume (100 μl) resulted in observation of vaccine virus in the lungs of all the animals at day 6, which was only observed for one animal inoculated with the smaller volume of 10 μl.

To evaluate PIV5 permissiveness in African green monkeys, 60 animals were screened for anti-PIV5 antibodies and all were found to be negative. Animals (n=3 per group) were infected intranasally with 1×10² up to 1×10⁸ PFU of PIV5 in 0.25 mL dose volume. Nasal wash and bronchoalveolar washes were assessed for virus shedding on days 3, 5, 7, 10 and 14. Virus was shed from both nose and lungs for up to 10-day period with peak replication at Day 5 for the dose as low as 1×10² PFU. This data demonstrates permissiveness of African green monkeys for PIV5.

Immunogenicity in cotton rats and African green monkeys after single dose control: Cotton rats were immunized intranasally with 1×10³, 1×10⁴, 1×10⁵ and 1×10⁶ PFU of W3A-RSV-F (SH-HN). At all dose levels, IgG antibody responses were noted at Day 28 with comparable responses per dose group. Neutralizing antibodies were observed in all dose group ranging from titer of 64 to 256 (NT50%). In addition, IgA responses in lung homogenates were observed in all dose groups at Day 21 post inoculation.

African green monkeys (PIV5 and RSV serum negative) received a single intranasal immunization with 1×10⁴ or 1×10⁶ PFU W3A-RSV-F (SH-HN). Sera obtained at Day 21 post inoculation showed high titers of F-specific antibody responses. In addition, neutralizing antibody responses were observed at Day 21 at low levels (NT50%, 52 in 1×10⁶ PFU dose group). Nasal swabs obtained at 21 days post immunization showed significant levels of F-protein IgA responses. Cell mediated responses as assessed by gamma interferon showed low level responses in the 1×10⁶ PFU dose group.

Protection in cotton rats and African green monkeys from RSV challenge: Cotton rats immunized intranasally with single dose ranging from 1×10³ to 1×10⁶ PFU W3A-RSV-F (SH-HN) were challenged with RSV A2 strain at Day 28 after immunization. Protection was assessed by measuring of viral loads in nose and lung tissues. Reduced viral loads were observed in a dose-dependent manner except for the 1×10⁶ PFU group which showed higher levels relative to 1×10³ PFU group but still reduced compared to control group. In the 1×10⁵ dose group, no virus was detected in the lung and reduced titers (order of 3 log₁₀) were observed in the nose.

African green monkeys immunized with 1×10⁴ or 1×10⁶ PFU W3A-RSV-F (SH-HN) were challenged 28 days after immunization with RSV A2. Nasal and BAL samples were assessed for RSV viral load Day 3-14 after challenge. Immunization with control did not shorten virus shedding, however, peak viral RSV loads were reduced 10 to 100-fold in both dose groups, with the highest reduction observed in the 1×10⁶ PFU dose group.

Responses to control in RSV exposed African green monkeys: Prior exposure to RSV did not interfere with ability to boost RSV neutralization antibody titers (50-fold increase) in African green monkeys (seroconverted by intranasal infection with RSV A2).

Lung pathology in W3A-RSV-F (SH-HN) immunized cotton rats after RSV challenge: In animals immunized at Day 0 with dose 1×10⁶ PFU W3A-RSV-F and challenged at Day 49 with RSV A2, lungs obtained five days after challenge were blindly examined by histopathology for alveolitis, interstitial pneumonitis, perivasculitis, and peribroncholitis and compared to lungs obtained from control animals inoculated with Formalin-inactivated RSV. The scores of positive control animals inoculated with formalin-inactivated RSV were significantly higher than PBS control animals but not the scores of W3A-RSV-F (SH-HN) animals (ANOVA paired t-test).

### B. Parainfluenza Virus 5 (PIV5)

Parainfluenza virus 5 (PIV5), a negative-stranded RNA virus, is a member of the Rubulavirus genus of the family Paramyxoviridae which includes many important human and animal pathogens such as mumps virus, human parainfluenza virus type 2 and type 4, Newcastle disease virus, Sendai virus, HPIV3, measles virus, canine distemper virus, rinderpest virus and respiratory syncytial virus. PIV5 was previously known as Simian Virus-5 (SV5). Although PIV5 is a virus that infects many animals and humans, no known symptoms or diseases in humans have been associated with PIV5. Unlike most paramyxoviruses, PIV5 infect normal cells with little cytopathic effect. As a negative stranded RNA virus, the genome of PIV5 is very stable. As PIV5 does not have a DNA phase in its life cycle and it replicates solely in cytoplasm, PIV5 is unable to integrate into the host genome. Therefore, using PIV5 as a vector avoids possible unintended consequences from genetic modifications of host cell DNAs. PIV5 can grow to high titers in cells, including Vero cells which have been approved for vaccine production by WHO and FDA. Thus. PIV5 presents many advantages as a vaccine vector.

A PIV5-based vaccine vector of the present invention may be based on any of a variety of wild type, mutant, or recombinant (rPIV5) strains. Wild type strains include, but are not limited to, the PIV5 strains W3A, WR (ATCC® Number VR-288™), canine parainfluenza virus strain 78-238 (ATCC number VR-1573) (Evermann et al., 1980, J Am Vet Med Assoc; 177:1132-1134; and Evermann et al., 1981, Arch Virol; 68:165-172), canine parainfluenza virus strain D008 (ATCC number VR-399) (Binn et al., 1967, Proc Soc Exp Biol Med; 126:140-145), MIL, DEN, LN, MEL, cryptovirus, CPI+, CPI−, H221, 78524, T1 and SER. See, for example, Chatziandreou et al., 2004, J Gen Virol; 85(Pt 10):3007-16; Choppin, 1964, Virology: 23:224-233; and Baumgartner et al., 1987, Intervirology; 27:218-223. Additionally, PIV5 strains used in commercial kennel cough vaccines, such as, for example, BI, F). Merck, and Merial vaccines, may be used.

### C. PIV5 CPI Strain Vector Backbone

The PIV5 CPI strain vector backbone differs from that of PIV5 W3A strain vector as follows: the most notable difference is in the PIV5 F protein of the CPI strain that consists of an additional 22 amino acid extension as part of its cytoplasmic tail. The extension of the F protein is thought to result in inhibition of the fusogenic properties of the virus (5,6). CPI based viruses are more lytic and produce more progeny virus in infected cells compared to the W3A based viruses that does not have the extended PIV5 F protein tail and possess additional amino acid difference compared with CPI (4).

i. PIV5 CPI Vectored RSV Constructs

A PIV5 vaccine vector of the present invention may be constructed using any of a variety of methods, including, but not limited to, the reverse genetics system described in more detail in He et al. (Virology; 237(2):249-60, 1997). PIV5 encodes eight viral proteins. Nucleocapsid protein (NP), phosphoprotein (P) and large RNA polymerase (L) protein are important for transcription and replication of the viral RNA genome. The V protein plays important roles in viral pathogenesis as well as viral RNA synthesis. The fusion (F) protein, a glycoprotein, mediates both cell-to-cell and virus-to-cell fusion in a pH-independent manner that is essential for virus entry into cells. The structures of the F protein have been determined and critical amino acid residues for efficient fusion have been identified. The hemagglutinin-neuraminidase (HN) glycoprotein is also involved in virus entry and release from the host cells. The matrix (M) protein plays an important role in virus assembly and budding. The hydrophobic (SH) protein is a 44-residue hydrophobic integral membrane protein and is oriented in membranes with its N terminus in the cytoplasm. For reviews of the molecular biology of paramyxoviruses see, for example, Whelan et al., 2004, Cuff Top Microbiol Immunol; 283:61-119; and Lamb & Parks, (2006). Paramyxoviridae: the viruses and their replication. In Fields Virology, 5th edn, pp. 1449-1496. Edited by D. M. Knipe & P. M. Howley. Philadelphia, PA: Lippincott Williams & Wilkins.

Previously, recombinant PIV5 viruses expressing foreign genes from numerous pathogens, including Influenza, Rabies, Respiratory Syncytial Virus, Tuberculosis, Burkholderia, and MERS-CoV have been generated and tested as vaccine candidates (Li, Z., et al., J Virol. 87(1):354 (2013); Chen, Z., et al., J Virol, 87(6): 2986 (2013); Wang, D., et al., J Virol, 91(11) (2017); Chen, Z., et al., Vaccine, 33(51):7217 (2015): Lafontaine, E. R., et al., Vaccine X., 1:100002 (2018); Li. K., et al., mBio. 11(2) (2020)). Because it actively replicates in the respiratory tract following intranasal immunization, PIV5-vectored vaccines can generate mucosal immunity that includes antigen-specific IgA antibodies and long-lived IgA plasma cells (Wang, D., et al., J Virol, 91(11) (2017). Xiao, P., et al., Front Immunol, 12:623996 (2021)).

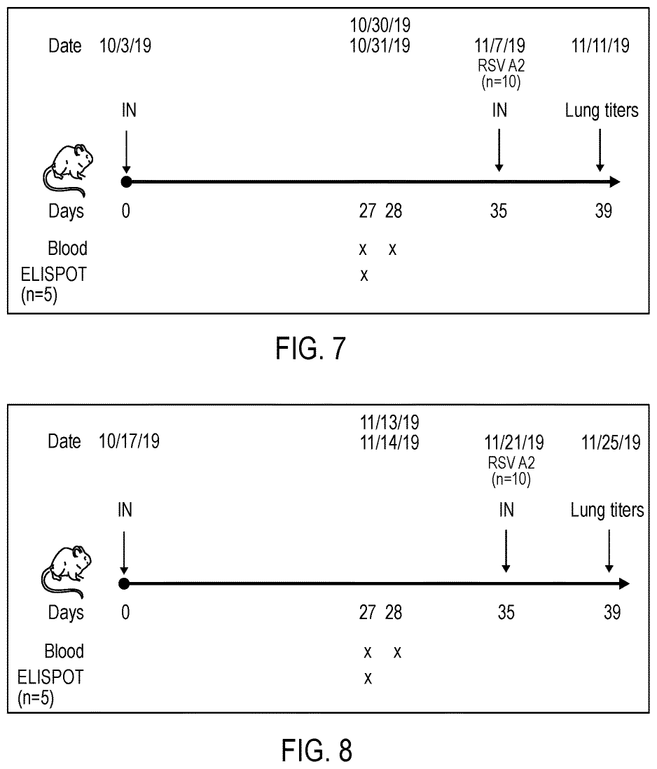

In one embodiment, The CPI-RSV-F vaccine drug substance (CPI-RSV-F) presented herein comprises a live recombinant PIV5 vectored virus based on the CPI strain of PIV5 expressing the wild type F-protein of RSV (F protein sequence based on GenBank Accession FJ614814J. SV A2 strain) codon-optimized for expression in human (FIG. 7). The wild type RSV F protein is inserted between the PIV5 SH and NH region (FIG. 1).

ii. Immunogenicity and Challenge Studies with CPI-RSV-F Vaccine Construct

In the non-clinical studies, a single dose of CPI-RSV-F was found to be immunogenic in mice, cotton rats, and AGMs following a single i.n. dose at dose levels ranging from 10⁴ to 10⁶ PFU. Both F-specific serum immune responses and cell-mediated responses were detected. The ability of the PIV5 vector to replicate in cotton rats and AGM was confirmed.

A single i.n. dose of CPI-RSV-F or related vaccine constructs was capable of protection from infection in various animal challenge models (mice, cotton rats and AGMs), using RSV A2 as challenge. Efficacy was based on reduced viral load observed in lung and nasal washes following challenge compared to viral load observed in control animals.

No observations of enhanced disease/lung pathology were observed in the cotton rat challenge model with CPI-RSV-F vector construct intended for use in the proposed Phase 1 study CPI-RSV-F. This conclusion was based on observed cytokine profile in lung tissue (no increase in IL-4 response) compared to positive control animals that had been vaccinated with formalin inactivated RSV (FI-RSV) and challenged with RSV. Similarly, no signs of enhanced disease/lung pathology were observed in the earlier published challenge studies in mice, cotton rats and AGM evaluating various W3A based vector constructs with F protein inserted at different locations within PIV5 W3A.

CPI-RSV-F was chosen over W3A for initial clinical evaluation given the similarity in being able to induce protective immune responses at similar dose levels as the W3A construct (albeit that immune responses to W3A appeared slightly higher). In addition, this same CPI PIV5 backbone engineered to express S-protein of SARS-CoV-2 is also being evaluated clinically under IND 027418 which is being cross-referenced. Preclinical data and limited clinical data obtained to date with this related vector construct show a favorable safety profile when administered as a single intranasal dose at 10⁶ PFU to healthy adults 18-55.

### III. BLB Genome Sequences

Provided herein are the CPI-RSV-F genomic sequences.

A. CPI-RSV-F Genomic sequence
i. CPI-RSV-F 5′ to 3′
The CPI-RSV-F 5′-3′ nucleic acid sequence is
provided herein.
(SEQ ID NO: 1)
ACCAGGGGGAAAACGAAGTGGTGACTCAAATCATCGAAGACCCTCGAGATT
ACATAGGTCCGGAACCTATGGCCTTCGTGACCGACCTCGAGTCAGAGTAG
TTCAATAAGGACCTATCAAGTTTGGGCAATTTTTCGTCCCCGACACAAAA
ATGTCATCCGTGCTTAAAGCATATGAGCGATTCACACTCACTCAAGAACT
GCAAGATCAGAGTGAGGAAGGTACAATCCCACCTACAACACTAAAACCGG
TAATCAGGGTATTTATACTAACCTCTAATAACCCAGAGCTAAGATCCCGG
CTTCTTCTATTTTGCCTACGGATTGTTCTCAGTAATGGTGCAAGGGATTC
CCATCGCTTTGGAGCATTACTTACAATGTTTTCGCTACCATCAGCCACAA
TGCTCAATCATGTCAAATTAGCTGACCAGTCACCAGAAGCTGATATCGAA
AGGGTAGAGATCGATGGCTTTGAGGAGGGATCATTCCGCTTAATCCCCAA
TGCTCGTTCAGGTATGAGCCGTGGAGAGATCAATGCCTATGCTGCACTTG
CAGAAGATCTACCTGACACACTAAACCATGCAACACCTTTTGTTGATTCC
GAAGTCGAGGGAACTGCATGGGATGAGATTGAGACTTTCTTAGATATGTG
TTACAGTGTCCTAATGCAGGCATGGATAGTGACTTGCAAGTGCATGACTG
CGCCAGACCAACCTGCTGCTTCTATTGAGAAACGCCTGCAAAAATATCGT
CAGCAAGGCAGGATCAACCCGAGATATCTCCTGCAACCGGAGGCTCGAAG
AATAATCCAGAATGTAATCCGAAAGGGAATGGTGGTCAGACATTTCCTCA
CCTTTGAACTGCAGCTTGCCCGAGCACAAAGCCTTGTATCAAATAGGTAT
TATGCTATGGTAGGGGATGTTGGAAAGTATATAGAGAATTGTGGAATGGG
AGGCTTCTTTTTGACACTAAAATATGCATTAGGAACCAGATGGCCCACAC
TTGCTTTAGCTGCATTTTCAGGAGAGCTAACAAAGCTAAAGTCCCTCATG
GCATTATACCAGACCCTTGGTGAGCAGGCCCGATATTTGGCCCTATTGGA
GTCACCACATTTGATGGATTTTGCTGCAGCAAACTACCCACTGCTATATA
GCTATGCTATGGGAATAGGCTATGTGTTAGATGTCAACATGAGGAACTAC
GCTTTCTCCAGATCATACATGAATAAGACATATTTCCAATTGGGAATGGA
AACTGCAAGAAAACAACAGGGTGCAGTTGACATGAGGATGGCAGAAGATC
TCGGTCTAACTCAAGCCGAACGCACCGAGATGGCAAATACACTTGCCAAA
TTGACCACAGCAAATCGAGGGGCAGACACCAGGGGAGGAGTCAACCCGTT
CTCATCTATCACTGGGACAACTCAGGTGCCCGCTGCAGCAACAGGTGACA
CATTCGAGAGTTACATGGCAGCGGATCGACTGAGGCAGAGATATGCTGAT
GCAGGCACCCACGATGATGAGATGCCACCATTGGAAGAGGAGGAAGAGGA
CGACACATCTGCAGGTCCACGCACTGGACTAACTCTTGAACAAGTGGCCT
TGGACATCCAGAACGCAGCAGTTGGAGCTCCCATCCATACAGATGACCTG
AATGCCGCACTGGGTGATCTTGACATCTAGACAATTCAGATCCCAATCTT
AAATCGACACACCTAATTGACCAGTTAGATGGAACTACAGTGGATTCCAT
AAGGTTCCTGCCTACCATCGGCTTTTAAGAAAAAAATAGGCCCGGACGGG
TTAGCAACAAGCGACTGCCGATGCCAATAACACAATCCACAATCTACAAT
GGATCCCACTGATCTGAGCTTCTCCCCAGATGAGATCAATAAGCTCATAG
AGACAGGCCTGAATACTGTGGAGTATTTTACTTCCCAACAAGTCACAGGA
ACATCCTCTCTTGGAAAGAATACAATACCACCAGGGGTCACAGGACTACT
AACCAATGCTGCAGAGGCAAAGATCCAAGAGTCAACCAACCATCAGAAGG
GTTCAGTTGGTGGGGGCGCAAAACCAAAGAAACCGCGACCAAAAATTGCC
ATTGTGCCAGCAGATGACAAAACGGTGCCCGGAAAGCCGATCCCAAACCC
TCTATTAGGTCTGGACTCCACCCCGAGCACCCAAACTGTGCTTGATCTAA
GTGGGAAAACATTACCATCAGGATCCTATAAGGGGGTTAAACTTGCGAAA
TTTGGAAAAGAAAATCTGATGACACGGTTCATCGAGGAACCCAGAGAGAA
TCCTATCGCAACCAATTCCCCCATCGATTTTAAGAGGGGCAGGGATACCG
GCGGGTTCCATAGAAGGGAGTACTCAATCGGATGGGTGGGAGATGAAGTC
AAGGTCACTGAGTGGTGCAATCCATCCTGTTCTCCAATCACCGCTGCAGC
AAGGCGATTTGAATGCACTTGTCACCAGTGTCCAGTCACTTGCTCTGAAT
GTGAACGAGATACTTAATACAGTGAGAAATTTGGACTCTCGGATGAATCA
ACTGGAGACAAAAGTAGATCGCATTCTCTCATCTCAGTCTCTAATCCAGA
CCATCAAGAATGACATAGTTGGACTTAAAGCAGGGATGGCTACTTTAGAA
GGAATGATTACAACTGTGAAAATCATGGACCCGGGAGTTCCCAGTAATGT
TACTGTGGAAGATGTACGCAAGAAACTAAGTAACCATGCTGTTGTTGTGC
CAGAATCATTCAATGATAGTTTCTTGACTCAATCTGAAGATGTAATTTCA
CTTGATGAGTTGGCTCGACCAACTGCAACAAGTGTTAAGAAGATTGTCAG
GAAGGTTCCTCCTCAGAAGGATCTGACTGGATTGAAGATCACACTAGAGC
AATTGGCAAAGGATTGCATCAGCAAACCGAAGATGAGGGAAGAGTATCTC
CTCAAAATCAACCAGGCTTCCAGTGAGGCTCAGCTAATTGACCTCAAGAA
AGCAATCATCCGCAGTGCAATTTGATCAAGAAACACCCAATTACACTACA
CTGGTATGACACTGTACTAACCCTGAGGGTTTTAGAAAAAACGATTAACG
ATAAATAAGCCCGAACACTACACACCACCCGAGGCAGCCATGCCATCCAT
CAGCATCCCCGCAGACCCCACCAATCCACGTCAATCAATAAAAGCGTTCC
CAATTGTGATCAACCGTGATGGGGGTGAGAAAGGTCGCTTGGTTAAACAA
CTACGCACAACCTACTTGAATGACCTAGATACTCATGAGCCACTGGTGAC
ATTCGTAAATACTTATGGATTCATCTACGAACAGGATCGGGGAAATACCA
TTGTCGGAGAGGATCAACATGGGAAGAAAAGAGAGGCTGTGACTGCTGCA
ATGGTTACCCTTGGATGTGGGCCTAATCTACCATCATTAGGGAATGTCCT
GGGACAACTGAGTGAATTCCAGGTCATTGTTAGGAAGACATCCAGCAAAG
CGGAAGAGATGGTCTTTGAAATTGTTAAGTATCCGAGAATATTTCGGGGT
CATACATTAATCCAGAAAGGACTAGTCTGTGTCTCCGCAGAAAAATTTGT
TAAGTCACCAGGGAAAGTACAATCTGGAATGGACTATCTCTTCATTCCGA
CATTTCTGTCAGTGACTTACTGTCCAGCTGCAATCAAATTTCAGGTACCT
GGCCCCATGTTGAAAATGAGATCAAGATACACTCAGAGCTTACAACTTGA
ACTAATGATAAGAATCCTGTGTAAGCCCGATTCGCCACTTATGAAGGTCC
ATATCCCTGACAAGGAAGGAAGAGGATGTCTTGTATCAGTATGGTTGCAT
GTATGCAATATCTTCAAATCAGGAAACAAGAATGGCAGTGAGTGGCAGGA
ATACTGGATGAGAAAGTGTGCTAACATGCAACTTGAAGTGTCGATTGCAG
ATATGTGGGGACCAACTATCATAATTCATGCCAGAGGTCACATTCCCAAA
AGTGCTAAGTTGTTTTTTGGAAAGGGTGGATGGAGCTGCCATCCACTTCA
CGAAGTTGTTCCAAGTGTCACTAAAACACTATGGTCCGTGGGCTGTGAGA
TTACAAAGGCGAAGGCAATAATACAAGAGAGTAGCATCTCTCTTCTCGTG
GAGACTACTGACATCATAAGTCCAAAAGTCAAAATTTCATCTAAGCATCG
CCGCTTTGGGAAATCAAATTGGGGTCTGTTCAAGAAAACCAAATCACTGC
CCAACCTGACGGAGCTGGAATGACTGACCCCCAATCGAGACTACACCACC
TCAAACTATAGGTGGGTGGTACCTCAGTGATTAATCTCGTAAGCACTGAT
CGTAGGCTACAACACACTAATATTATCCAGATTAGAGAGCTTAATTAGCT
CTGTATTAATAATAACACTACTATTCCAATAACTGGAATCACCAGCTTGA
TTTATCTCCAAAATGATTCAAAGAAAACAAATCATATTAAGACTATCCTA
AGCACGAACCCATATCGTCCTTCAAATCATGGGTACTATAATTCAATTTC
TGGTGGTCTCCTGTCTATTGGCAGGAGCAGGCAGTCTTGATCCAGCAGCC
CTCATGCAAATCGGTGTCATTCCAACAAATGTCCGGCAACTTATGTATTA
TACTGAGGCCTCATCAGCATTCATTGTTGTGAAGTTAATGCCTACAATTG
ACTCGCCGATTAGTGGATGTAATATAACATCAATTTCAAGCTATAATGCA
ACAGTGACAAAACTCCTACAGCCGATCGGTGAGAATTTGGAGACGATTAG
GAACCAGTTGATTCCAACTCGGAGGAGACGCCGGTTTGCAGGGGTGGTGA
TTGGATTAGCTGCATTAGGAGTAGCTACTGCCGCACAGGTCACTGCCGCA
GTAGCACTAGTAAAGGCAAATGAAAATGCTGCGGCTATACTCAATCTCAA
AAATGCAATCCAAAAAACAAATGCAGCAGTTGCAGATGTGGTCCAGGCCA
CACAATCACTAGGAACaGCAGTTCAAGCAGTTCAAGATCACATAAACAGT
GTGGTAAGTCCAGCAATTACAGCAGCCAATTGTAAAGCCCAAGATGCTAT
CATTGGCTCAATCCTCAATCTCTATTTGACCGAGTTGACAACTATCTTCC
ACAATCAAATTACAAACCCTGCATTGAGTCCTATTACAATTCAAGCTTTA
AGGATCCTACTAGGGAGTACCTTGCCGACTGTGGTCGAAAAATCTTTCAA
TACCCAGATAAGTGCGGCTGAGCTTCTCTCATCAGGGTTATTGACAGGCC
AGATTGTGGGATTAGATTTGACCTATATGCAGATGGTCATAAAAATTGAG
CTGCCAACTTTAACTGTACAACCTGCAACCCAGATCATAGATCTGGCCAC
CATTICTGCATTCATTAACAATCAAGAAGTCATGGCCCAATTACCAACAC
GTGTTATTGTGACTGGCAGCTTGATCCAAGCCTATCCCGCATCGCAATGC
ACTATTACACCCAACACTGTGTACTGTAGGTATAATGATGCCCAAGTACT
CTCAGATGATACGATGGCTTGCCTCCAAGGTAACTTGACAAGATGCACCT
TCTCTCCAGTGGTTGGGAGCTTTCTCACTCGATTCGTGCTGTTCGATGGA
ATAGTTTATGCAAATTGCAGGTCGATGTTGTGCAAGTGCATGCAGCCTGC
TGCTGTGATCCTACAGCCGAGTTCATCCCCTGTAACTGTCATTGACATGT
ACAAATGTGTGAGTCTGCAGCTTGACAATCTCAGATTCACCATCACTCAA
TTGGCCAATGTAACCTACAATAGCACCATCAAGCTTGAAACATCCCAGAT
CTTGCCTATTGATCCGTTGGATATATCCCAGAATCTAGCTGCGGTGAATA
AGAGTCTAAGTGATGCACTACAACACTTAGCACAAAGTGACACATACCTT
TCTGCAATCACATCAGCTACGACTACAAGTGTATTATCCATAATAGCAAT
CTGTCTTGGATCGTTAGGTTTAATATTAATAATCTTGCTCAGTGTAGTTG
TGTGGAAGTTATTGACCATTGTCGCTGCTAATCGAAATAGAATGGAGAAT
TTTGTTTATCATAATTCAGCATTCCACCACTCACGATCTGATCTCAGTGA
GAAAAATCAACCTGCAACTCTTGGAACAAGATAAGACAGTCATCCATTAG
TAATTTTTAAGAAAAAAACGATAGGACCGAAACTAGTATTGAAAGAACCG
TCTCGGTCAATCTAGGTAATCGAGCTGcTACCGTCTCGGAAAGCTCAAAT
CATGCTGCCTGATCCGGAAGATCCGGAAAGCAAAAAAGCTACAAGGAGAA
CAGGAAACCTAATTATCTGCTTCCTATTCATCTTCTTTCCGTTTGTAAAC
TTCATTGTTCCAACTCTAAGACACTTGCTGTCCTAACACCTGCTATAGGC
TATCCACTGCATCATCTCTTTTTAAGAAAAAAATAGGCCCGGACGGGTTA
GCAACAAGCGACTGCCGGTGCCAACAACACAATCCACAATCTACAGTCGA
CGCGGCCGCCACCATGGAGCTGCTGATCCTGAAAGCCAACGCTATTACCA
CAATCCTGACCGCCGTGACATTCTGCTTTGCTTCCGGACAGAATATCACC
GAGGAGTTCTACCAGTCAACATGTAGCGCCGTGAGCAAGGGGTACCTGAG
TGCACTGCGAACCGGATGGTATACATCCGTGATTACTATCGAGCTGTCTA
ACATCAAGAAAAACAAATGCAATGGCACTGACGCCAAGATCAAGCTGATC
AAGCAGGAGCTGGATAAGTACAAAAATGCTGTGACCGAACTGCAGCTGCT
GATGCAGAGCACTCCAGCTACCAACAATCGCGCACGGAGAGAACTGCCCC
GGTTCATGAACTATACCCTGAACAATGCAAAGAAAACAAATGTGACTCTG
TCCAAGAAACGCAAGAGGCGCTTCCTGGGATTTCTGCTGGGCGTGGGGTC
TGCAATCGCCAGTGGAGTGGCCGTCTCTAAAGTCCTGCACCTGGAGGGCG
AAGTGAACAAGATCAAATCAGCACTGCTGAGCACAAACAAGGCCGTGGTC
AGTCTGTCAAATGGCGTGTCCGTCCTGACTTCTAAGGTGCTGGACCTGAA
AAATTATATTGATAAGCAGCTGCTGCCTATCGTCAACAAACAGAGCTGTT
CCATTTCTAATATCGAGACCGTGATCGAGTTCCAGCAGAAGAACAATAGA
CTTCTAGAGATTACAAGGGAATTTTCTGTGAACGCTGGCGTCACTACCCC
CGTGAGTACCTACATGCTGACAAATAGTGAGCTGCTGTCACTGATTAACG
ACATGCCTATCACCAATGATCAGAAGAAACTGATGAGTAACAATGTGCAG
ATCGTCAGACAGCAGAGTTACTCAATCATGTCAATCATTAAGGAGGAAGT
CCTGGCCTACGTGGTCCAGCTGCCACTGTATGGCGTGATCGACACTCCCT
GCTGGAAACTGCATACCTCACCTCTGTGCACAACTAACACCAAGGAGGGG
AGCAATATCTGCCTGACACGAACTGACCGGGGATGGTACTGTGATAACGC
CGGCAGCGTGTCCTTCTTTCCCCAGGCTGAGACCTGCAAGGTCCAGTCCA
ACAGGGTGTTCTGTGACACTATGAATAGCCTGACCCTGCCTTCCGAAGTC
AACCTGTGCAATGTGGACATCTTTAATCCAAAGTACGATTGTAAGATCAT
GACCAGCAAGACAGATGTCAGCTCGAGTGTGATTACATCACTGGGGGCTA
TCGTGAGCTGCTACGGAAAGACTAAATGTACCGCAAGCAACAAAAATCGC
GGCATCATTAAGACCTTCAGTAACGGGTGCGACTATGTCTCAAACAAGGG
CGTGGATACAGTGAGCGTCGGGAACACTCTGTACTATGTCAATAAGCAGG
AGGGAAAATCCCTGTACGTGAAGGGCGAACCAATCATTAACTTCTATGAC
CCCCTGGTGTTCCCTTCCGACGAGTTTGATGCCTCTATTAGTCAGGTGAA
CGAAAAAATCAATCAGAGCCTGGCTTTTATTCGGAAGTCCGATGAGCTGC
TGCACAACGTGAATGCAGTCAAGTCTACCACAAACATTATGATCACTACC
ATCATTATCGTGATTATCGTCATTCTGCTGTCCCTGATCGCAGTGGGGCT
GCTGCTGTACTGTAAAGCCAGATCTACACCCGTGACTCTGTCTAAGGATC
AGCTGAGTGGAATTAACAATATCGCCTTTAGCAACTGATAGGCTAGCCTC
CTGCCATACTTCCTACTCACATCATATCTATTTTAAAGAAAAAATAGGCC
CGAACACTAATCGTGCCGGCAGTGCCACTGCACACACAACACTACACATA
CAATACACTACAATGGTTGCAGAAGATGCCCCTGTTAGGGGCACTTGCCG
AGTATTATTTCGAACAACAACTTTAATTTTTCTATGCACACTATTAGCAT
TAAGCATCTCTATCCTTTATGAGAGTTTAATAACCCAAAAGCAAATCATG
AGCCAAGCAGGCTCAACTGGATCTAATTCTAGATTAGGAAGTATCACTGA
TCTTCTTAATAATATTCTCTCTGTCGCAAATCAGATTATATATAACTCTG
CAGTCGCTCTACCTCTACAATTGGACACTCTTGAATCAACACTCCTTACA
GCCATTAAGTCTCTTCAAACAAGTGACAAGCTAGAACAGAACTGCTCGTG
GGGTGCTGCACTGATTAATGATAATAGATACATTAATGGCATCAATCAGT
TCTATTTCTCAATTGCTGAGGGTCGCAAGCTGACACTTGGCCCACTTCTT
AATATACCTAGTTTCATTCCAACTGCCACGACACCAGAGGGCTGCACCAG
GATCCCATCATTCTCGCTCACCAAGACACACTGGTGTTATACACACAATG
TTATCCTGAATGGATGCCAGGATCATGTATCCTCAAATCAATTTGTTTCC
ATGGGAATCATTGAACCCACTTCTGCCGGGTTTCCATCCTTTCGAACCCT
AAAGACTCTATATCTCAGCGATGGGGTCAATCGTAAGAGCTGCTCTATCA
GTACAGTTCCGGGGGGTTGTATGATGTACTGTTTCGTCTCTACTCAACCA
GAGAGGGATGACTACTTTTCTACCGCTCCTCCAGAACAACGAATTATTAT
AATGTACTATAATGATACAATCGTGGAGCGCATAATTAATCCACCCGGGG
TACTAGATGTATGGGCAACATTGAACCCAGGAACAGGAAGCGGGGTATAT
TATTTAGGTTGGGTACTCTTTCCAATATATGGCGGCGTGATTAAAGATAC
GAGTTTATGGAATAATCAAGCAAATAAATACTTTATCCCCCAGATGGTTG
CTGCTCTCTGCTCACAAAACCAGGCAACTCAAGTCCAAAATGCTAAGTCA
TCATACTATAGCAGCTGGTTTGGCAATCGAATGATTCAGTCTGGGATCCT
GGCATGTCCTCTTCAACAGGATCTAACCAATGAGTGTTTAGTTCTGCCCT
TTTCTAATGATCAGGTGCTTATGGGTGCTGAAGGGAGATTATACATGTAT
GGTGACTCGGTGTATTACTACCAAAGAAGCAATAGTTGGTGGCCTATGAC
CATGCTGTATAAGGTAACCATAACATTCACTAATGGTCAGCCATCTGCTA
TATCAGCTCAGAATGTGCCCACACAGCAGGTCCCTAGACCTGGGACAGGA
AGCTGCTCTGCAACAAATAGATGTCCCGGTTTTTGCTTGAAAGGAGTGTA
TGCTGATGCCTGGTTACTGACCAACCCTTCGTCTACCAGTACATTTGGAT
CAGAAGCAACCTTCACTGGTTCTTATCTCAACGCAGCAACTCAGCGTATC
AATCCGACGATGTATATCGCGAACAACACACAGATCATAAGCTCACAGCA
ATTTGGATCAAGCGGTCAAGAAGCAGCATATGGCCACACAACTTGTTTTA
GGGACACAGGCTCTGTTATGGTATACTGTATCTATATTATTGAATTGTCC
TCATCTCTCTTAGGACAATTTCAGATTGTCCCATTTATCCGTCAGGTGAC
ACTATCCTAAAGGCAGAAGCCTCCAGGTCTGACCCAGCCAATCAAAGCAT
TATACCAGACCATGGAATGCATACCAAACATTATTGACACTAATGACACA
CAAAATTGGTTTTAAGAAAAACCAAGAGAACAATAGGCCAGAATGGCTGG
GTCTCGGGAGATATTACTCCCTGAAGTCCATCTCAATTCACCAATTGTAA
AGCATAAGCTATACTATTACATTCTACTTGGAAACCTCCCAAATGAGATC
GACATTGACGATTTAGGTCCATTACATAATCAAAATTGGAATCAAATAGC
ACATGAAGAGTCTAACTTAGCCCAACGCTTGGTAAATGTAAGAAATTTTC
TAATTACCCACATCCCTGATCTTAGAAAGGGCCATTGGCAAGAGTATGTC
AATGTAATACTGTGGCCGCGAATTCTTCCCTTGATCCCGGATTTTAAAAT
CAATGACCAATTGCCTCTACTCAAAAATTGGGACAAGTTAGTTAAAGAAT
CATGTTCAGTAATCAATGCGGGTACTTCCCAGTGCATTCAGAATCTCAGC
TATGGACTGACAGGTCGTGGGAACCTCTTTACACGATCACGTGAACTCTC
TGGTGACCGCAGGGATATTGATCTTAAGACGGTTGTGGCAGCATGGCATG
ACTCAGACTGGAAAAGAATAAGTGATTTTTGGATTATGATCAAATTCCAG
ATGAGACAATTAATTGTTAGGCAAACAGATCATAATGATCCTGATTTAAT
CACGTATATCGAAAATAGAGAAGGCATAATCATCATAACCCCTGAACTGG
TAGCATTATTTAACACTGAGAATCATACACTAACATACATGACCTTTGAA
ATTGTACTGATGGTTTCAGATATGTACGAAGGTCGTCACAACATTTTATC
ACTATGCACAGTTAGCACTTACCTGAATCCTCTGAAGAAAAGAATAACAT
ATTTATTGAGCCTTGTAGATAACTTAGCTTTTCAGATAGGTGATGCTGTA
TATAACATAATTGCTTTGCTAGAATCCTTTGTATATGCACAGTTGCAAAT
GTCAGATCCCATCCCAGAACTCAGAGGACAATTCCATGCATTCGTATGTT
CTGAGATTCTTGATGCACTAAGGGGAACTAATAGTTTCACCCAGGATGAA
TCAAGAACTGTGACAACCAATTTGATATCCCCATTCCAAGATCTGACCCC
AGATCTTACGGCTGAATTGCTCTGTATAATGAGGCTTTGGGGACACCCCA
TGCTCACCGCCAGTCAAGCTGCGGGAAAGGTACGCGAGTCCATGTGTGCT
GGAAAAGTATTAGACTTTCCCACCATTATGAAAACACTAGCCTTTTTCCA
TACTATTCTGATCAATGGATACAGGAGGAAGCATCATGGAGTATGGCCAC
CCTTAAACTTACCGGGTAATGCTTCAAAGGGTCTCACGGAACTTATGAAT
GACAATACTGAGATAAGCTATGAATTCACACTTAAGCATTGGAAGGAAAT
CTCTCTTATAAAATTCAAGAAATGTTTTGATGCAGACGCAGGTGAGGAAC
TCAGTATATTTATGAAAGATAAAGCAATTAGTGCCCCAAAACAAGATTGG
ATGAGTGTGTTTAGAAGAAGCCTAATCAAACAGCGCCATCAGCATCATCA
GGTCCCCCTACCAAATCCATTCAATCGACGGCTATTGCTAAACTTTCTCG
GAGATGACAAATTCGACCCGAATGTGGAGCTACAGTATGTAACATCAGGT
GAGTATCTACATGATGACACGTTTTGTGCATCATATTCACTAAAAGAGAA
GGAAATTAAACCTGATGGTCGAATTTTTGCAAAGTTGACTAAGAGAATGA
GATCATGTCAAGTTATAGCAGAATCTCTTTTAGCGAACCATGCTGGGAAG
TTAATGAAAGAGAATGGTGTTGTGATGAATCAGCTATCATTAACAAAATC
ACTATTAACAATGAGTCAGATTGGAATAATATCCGAGAAAGCTAGAAAAT
CGACTCGAGATAACATAAATCAACCTGGTTTCCAGAATATCCAGAGAAAT
AAATCACATCACTCCAAGCAAGTCAATCAGCGAGATCCAAGTGATGACTT
TGAATTGGCAGCATCTTTTTTAACTACTGATCTCAAAAAATATTGTTTAC
AATGGAGGTACCAGACAATTATCCCATTTGCTCAATCATTAAACAGAATG
TATGGTTATCCTCATCTCTTTGAGTGGATTCACTTACGGCTAATGCGTAG
TACACTTTACGTGGGGGATCCCTTCAACCCACCAGCAGATACCAGTCAAT
TTGATCTAGATAAAGTAATTAATGGAGATATCTTCATTGTATCACCCAGA
GGTGGAATTGAAGGGCTATGTCAAAAGGCTTGGACAATGATATCTATCTC
TGTGATAATTCTATCTGCCACAGAGTCTGGCACACGAGTAATGAGTATGG
TGCAGGGAGATAATCAAGCAATTGCTGTCACCACACGAGTACCAAGGAGC
CTGCCGACTCTTGAGAAAAAGACTATTGCTTTTAGATCTTGTAATCTATT
CTTTGAGAGGTTAAAATGTAATAATTTTGGATTAGGTCACCATTTGAAAG
AACAAGAGACTATCATTAGTTCTCACTTCTTTGTTTATAGCAAGAGAATA
TTCTATCAGGGGAGGATTCTAACGCAAGCCTTAAAAAATGCTAGTAAGCT
CTGCTTGACAGCTGATGTCCTAGGAGAATGTACCCAATCATCATGTTCTA
ATCTTGCAACTACTGTCATGAGGTTAACTGAGAATGGTGTTGAAAAAGAT
ATCTGTTTCTACTTGAATATCTATATGACCATCAAACAGCTCTCCTATGA
TATCATCTTCCCTCAAGTGTCAATTCCTGGAGATCAGATCACATTAGAAT
ACATAAATAATCCACACCTGGTATCACGATTGGCTCTTCTGCCATCCCAG
CTAGGAGGTCTAAACTACCTGTCATGCAGTAGGCTGTTCAATCGAAACAT
AGGCGACCCGGTGGTTTCCGCAGTTGCAGATCTTAAGAGATTAATTAAAT
CAGGATGTATGGATTACTGGATCCTTTATAACTTATTAGGGAGAAAACCG
GGAAACGGCTCATGGGCTACTTTAGCAGCTGACCCGTACTCAATCAATAT
AGAGTATCAATACCCCCCAACTACAGCTCTTAAGAGGCACACCCAACAAG
CTCTGATGGAACTCAGTACGAATCCAATGTTACGTGGCATATTCTCTGAC
AATGCACAGGCAGAAGAAAATAATCTTGCTAGATTTCTCCTGGATAGGGA
GGTGATCTTTCCGCGTGTAGCTCACATCATCATTGAGCAAACCAGTGTCG
GGAGGAGAAAACAGATTCAAGGATATTTGGATTCAACTAGATCGATAATG
AGTAAATCACTAGAAATTAAGCCCTTGTCCAATAGGAAGCTTAATGAAAT
ACTGGATTACAACATCAATTACCTAGCTTACAATTTGGCATTACTCAAGA
ATGCTATTGAACCTCCGACTTATTTGAAAGCAATGACTCTTGAAACATGT
AGCATCGACATTGCAAGGAGCCTCCGGAAGCTCTCCTGGGCCCCACTCTT
GGGTGGGAGAAATCTTGAAGGATTAGAGACGCCAGATCCCATTGAAATTA
CTGCAGGAGCATTAATTGTTGGATCGGGCTACTGTGAACAGTGTGCTGCA
GGAGACAATCGATTCACATGGTTTTTCTTGCCATCTGGTATCGAGATAGG
AGGGGATCCCCGTGATAATCCTCCTATCCGTGTACCGTACATTGGCTCCA
GGACTGATGAGAGGAGGGTAGCCTCAATGGCATACATCAGGGGTGCCTCG
AGTAGCCTAAAAGCAGTTCTTAGACTGGCGGGAGTGTACATCTGGGCATT
CGGAGATACTCTGGAGAATTGGATAGATGCACTGGATTTGTCTCACACTA
GAGTTAACATCACACTTGAACAGCTGCAATCCCTCACCCCACTTCCAACC
TCTGCCAATCTAACCCATCGGTTGGATGATGGCACAACTACCCTAAAGTT
TACTCCTGCGAGCTCTTACACCTTTTCAAGTTTCACTCATATATCAAATG
ATGAGCAATACCTGACAATTAATGACAAAACTGCAGATTCAAATATAATC
TACCAACAGTTAATGATCACTGGACTCGGAATTTTAGAAACATGGAATAA
TCCCCCAATCAATAGAACATTCGAAGAATCTACCCTACATTTGCACACTG
GTGCATCATGTTGTGTCCGACCTGTGGACTCCTGCATCATCTCAGAAGCA
TTAACAGTCAAGCCACATATTACAGTACCGTACAGCAATAAATTTGTATT
TGATGAAGACCCGCTATCTGAATATGAGACTGCAAAACTGGAATCGTTAT
CATTTCAAGCCCAATTAGGCAACATTGATGCTGTAGATATGACAGGTAAA
TTAACATTATTGTCCCAATTCACTGCAAGGCAGATTATTAATGCAATCAC
TGGACTCGATGAGTCTGTCTCTCTTACTAATGATGCCATTGTTGCATCAG
ACTATGTCTCCAATTGGATTAGTGAATGCATGTATACCAAATTAGATGAA
TTATTTATGTATTGTGGGTGGGAACTACTATTGGAACTATCCTATCAAAT
GTATTATCTGAGGGTAGTTGGGTGGAGTAATATAGTGGATTATTCTTACA
TGATCTTGAGAAGAATCCCGGGTGCAGCATTAAACAATCTGGCATCTACA
TTAAGTCATCCAAAACTTTTCCGACGAGCTATCAACCTAGATATAGTTGC
CCCCTTAAATGCTCCTCATTTTGCATCTCTGGACTACATCAAGATGAGTG
TGGATGCAATACTCTGGGGCTGTAAAAGAGTCATCAATGTGCTCTCCAAT
GGAGGGGACTTAGAATTAGTTGTGACATCTGAAGATAGCCTTATTCTCAG
TGACCGATCCATGAATCTCATTGCAAGGAAATTAACTTTATTATCACTGA
TTCACCATAATGGTTTGGAACTACCAAAGATTAAGGGGTTCTCTCCTGAT
GAGAAGTGTTTCGCTTTGACAGAATTTTTGAGGAAAGTGGTGAACTCAGG
GTTGAGTTCAATAGAGAACCTATCAAATTTTATGTACAATGTGGAGAACC
CACGGCTTGCAGCATTCGCCAGCAACAATTACTACCTGACCAGAAAATTA
TTGAATTCAATACGAGATACTGAGTCGGGTCAAGTAGCAGTCACCTCATA
TTATGAATCATTAGAATATATTGATAGTCTTAAGCTAACCCCACATGTGC
CTGGTACCTCATGCATTGAGGATGATAGTCTATGTACAAATGATTACATA
ATCTGGATCATAGAGTCTAATGCAAACTTGGAGAAGTATCCAATTCCAAA
TAGCCCTGAGGATGATTCCAATTTCCATAACTTTAAGTTGAATGCTCCAT
CGCACCATACCTTACGCCCATTAGGGTTGTCATCAACTGCTTGGTATAAG
GGTATAAGCTGTTGCAGGTACCTTGAGCGATTAAAGCTACCACAAGGTGA
TCATTTATATATTGCAGAAGGTAGTGGTGCCAGTATGACAATCATAGAAT
ACCTATTCCCAGGAAGAAAGATATATTACAATTCTTTATTTAGTAGTGGT
GACAATCCCCCACAAAGAAATTATGCACCAATGCCTACTCAGTTCATTGA
GAGTGTCCCATACAAGCTCTGGCAAGCACACACAGATCAATATCCCGAGA
TTTTTGAGGACTTCATCCCTCTATGGAACGGAAATGCCGCCATGACTGAC
ATAGGAATGACAGCTTGTGTAGAATTCATCATCAATCGAGTCGGCCCAAG
GACTTGCAGTTTAGTACATGTAGATTTGGAATCAAGTGCAAGCTTAAATC
AACAATGCCTGTCAAAGCCGATAATTAATGCTATCATCACTGCTACAACT
GTTTTGTGCCCTCATGGGGTGCTTATTCTGAAATATAGTTGGTTGCCATT
TACTAGATTTAGTACTTTGATCACTTTCTTATGGTGCTACTTTGAGAGAA
TCACTGTTCTTAGGAGCACATATTCTGGTCCAGCTAATCATGAGGTTTAT
TTAATTTGTATCCTTGCCAACAACTTTGCATTCCAGACTGTCTCGCAGGC
AACAGGAATGGCGATGACTTTAACCGATCAAGGGTTTACTTTGATATCAC
CTGAAAGAATAAATCAGTATTGGGATGGTCACTTGAAGCAAGAACGTATC
GTAGCAGAAGCAATTGATAAGGTGGTTCTAGGAGAAGATGCTCTATTCAA
TTCGAGTGATAATGAATTAATTCTCAAATGTGGAGGGACACCAAATGCAC
GGAATCTTATCGATATCGAGCCAGTCGCAACTTTCATAGAATTTGAACAA
CTGATCTGCACAATGTTGACAACCCACTTGAAGGAAATAATTGATATAAC
AAGGTCTGGAACCCAGGATTATGAAAGTTTATTACTCACTCCTTACAATT
TAGGTCTTCTTGGTAAAATCAGTACGATAGTGAGATTATTAACAGAAAGG
ATTCTAAATCATACTATCAGGAATTGGTTGATCCTCCCACCTTCGCTCCG
GATGATCGTGAAGCAGGACTTGGAATTCGGCATATTCAGGATTACTTCCA
TCCTCAATTCTGATCGGTTCCTGAAGCTTTCTCCAAATAGGAAATACTTG
ATTACACAATTAACTGCAGGCTACATTAGGAAATTGATTGAGGGGGATTG
TAATATCGATCTAACCAGACCTATCCAAAAACAAATCTGGAAAGCATTAG
GTTGTGTAGTCTATTGTCACGATCCAGTAGATCAAAGGGAATCAACAGAG
TTTATTGATATAAATATTAATGAAGAAATAGACCGCGGGATCGATGGCGA
GGAAATCTAAATATATCAAGAATCAGAATTAGTTTAAGAAAAAAGAAGAG
GATTAATCTTGGTTTTCCCCTTGGT

### IV. Methods of Treatment

The disclosure can be used in gene therapy and/or therapeutic approaches for the treatment of disease which involve the increase or decrease of a nucleotide sequence of interest in a host-cell. In these embodiments, the expressible heterologous nucleotide sequence may be derived from a mammalian genome. It may be particularly useful in some embodiments to have the expressible heterologous nucleotide sequence derived from a human genome, wherein expression of the wild-type RNA and/or protein can produce therapeutic effects in a patient. For example, the expressible heterologous nucleotide sequence can encode CFTR, NeuroD1, Cas9 and Guide RNAs, or any other such sequence. In other embodiments, the heterologous nucleotide sequence encodes a secreted protein.

In other embodiments, the expressible heterologous nucleotide sequence responds to positive selection stimuli. In other embodiments, the expressible heterologous nucleotide sequence also responds to negative selection stimuli. In further embodiments, it may be useful for the polynucleotide sequences to further comprise a reporter gene. For example, the report gene can be a luciferase or green fluorescent protein.

### V. Methods of Administration

### A. Administration by Vaccination

The present invention includes methods of vaccinating a subject by administering a viral expression vector, viral particle, or composition as described herein to the subject.

The invention provides the use of a live recombinant PIV5 based vaccine consisting of canine parainfluenza (CPI) engineered to express the RSV F protein as target antigen to develop a CPI-RSV-F vaccine as a novel prophylactic vaccine against RSV infection.

i. Intranasal Vaccination

In one embodiment, the disclosed CPI-RSV-F compositions are formulated to allow intranasal administration. A number of non-clinical studies have been conducted to evaluate the immunogenicity and efficacy of CPI-RSV-F or very close related vaccine construct (e.g. W3A PIV5 based constructs) expressing RSV F protein in various animal models using the intranasal (i.n.) route. This included earlier developmental studies in mice, cotton rats and African green monkeys (AGM) evaluating variations of the vector construct (location of F protein in vector) and of the RSV F protein insert (wild type or prefusion F protein). The findings from these studies are summarized and the publications are hereby included by incorporation.

Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 μm. More particularly, the salt form of the therapeutic agent can have a particle size distribution for intranasal administration between about 5μ to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 μm, greater than about 38 μm, less than about 53 μm, less than about 70 μm, greater than about 100 μm, or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 μm, or about 2 μm and about 5 μm. In other embodiments, the salt form of the therapeutic agent has a mean particle size of at least 1 μm, at least 2 μm, at least 3 μm, at least 4 μm, at least 5 μm, at least 10 μm, at least 20 μm, at least 25 μm, at least 30 μm, at least 40 μm, at least 50 μm, at least 60 μm, at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

a. Vaccine Immunogenicity and Protection in Lower Respiratory Tract of Mice

One embodiment provides candidate vaccine viruses administered intranasally which elicited high levels of anti-F serum antibodies in both CHD03 and CHD04 studies. In W3A,SH—RSV-F vaccinated animals, the geometric mean titer of RSV F antibody titer was 3.1 Log₁₀/mL in CHD03 and 3.4 Log₁₀/mL in CHD04. In CPI-RSV-F vaccinated animals, the geometric mean titer was 3.0 Log₁₀/mL in CHDO3 and 3.2 Log₁₀/mL in CHD04. These values are not statistically significant from each other, and they are similar to the values from RSV_rA2 positive control group. Both vaccine viruses also elicited RSV F protein specific cellular responses as measured by IFN-γ secreting cells. In CHD03, W3AΔSH-RSV-F had a geometric mean value of 28 IFN-γ secreting cells while CPI-RSV-F had a geometric mean of 23 IFN-γ secreting cells per 10⁶ splenocytes. In CHD04, W3AΔSH-RSV-F and CPI-RSV-F had geometric means of 35 IFN-γ secreting cells and 48 IFN-γ secreting cells per 10⁶ splenocytes, respectively. These values were statistically significant from the PBS control group, but not different between the vaccinated groups or the RSV_rA2 positive control group. IFN-γ secreting cells in CPI-RSV-F group in CHD04 was significantly higher than positive control. Both W3AΔSH-RSV-F and CPI-RSV-F had significantly lower RSV challenge viral titers compared to PBS control group in both CHD03 and CHD04. The values were 1.35 Log₁₀ PFU/g and 1.37 Log₁₀ PFU/g for W3AΔSH-RSV-F and 1.48 Log₁₀ PFU/g and 1.40 Log₁₀ PFU/g CPI-RSV-F, respectively for CHD03 and CHD04 compared to 3.21 Log₁₀ PFU/g and 3.33 Log₁₀ PFU/g in the PBS control groups. The RSV_rA2 positive control group had values of 1.39 Log₁₀ PFU/g in CHD03 and 1.32 Log₁₀ PFU/g in CHD04, which is similar to the vaccine groups of interest.

Overall, the results presented herein demonstrate that W3AΔSH-RSV-F and CPI-RSV-F, as well as the other two candidates (W3A-RSV-F (SH-HN) and CPIΔSH-RSV-F), administered intranasally at a dose level of 10⁵ PFU, induced comparable RSV-specific immune responses and significant protection against RSV challenge virus replication in the lower respiratory tract of BALB/c mice.

b. Preclinical Testing of PIV5ΔSH-RSV-F Vaccine Based on CPI Strain of PIV5 in the Cotton Rat Virus Challenge Model Using RSV A/A2 Virus Strain. Comparison to PIV5 W3A-Based PIV5ΔSH-RSV-F Vaccine.

Efficacy and safety of RSV F protein vaccine candidates based on the CPI strain or the W3A strain of PIV5 (CPI-RSV-F and W3AΔSH-RSV-F, respectively) were evaluated in the cotton rat Sigmodon hispidus model of RSV A/A2 challenge. Animals were first immunized with 100 μl of the PIV5-based vaccines containing 10⁴, 10⁵, or 10⁶ PFU of virus intranasally and then challenged with 10⁵ PFU RSV A/A2 four weeks later. The primary infection control group was mock-immunized with PBS and then infected with RSV A/A2. The secondary infection control group was infected with RSV A/A2 and re-infected seven weeks later. The vaccine-enhanced disease control group was immunized with FI-RSV twice with an interval of four weeks and infected with RSV three weeks after the second immunization. Animals were sacrificed five days after RSV challenge for sample collection. RSV replication in the lung and nose, pulmonary histopathology, pulmonary cytokine and RSV NS1 mRNA expression, and the RSV serum neutralizing antibody (NA) and the anti-F protein binding antibody titers were measured. To confirm replication of the PIV5 vaccines in the respiratory tract of cotton rats, groups of 3 animals were inoculated with 10⁶ PFU of CPI—RSV-F or W3AΔSH-RSV-F intranasally and sacrificed four days later. These samples were submitted for plaque assay evaluation. In brief, it was found that both candidate vaccine viruses replicated efficiently in the upper and lower respiratory track of cotton rats. W3AΔSH-RSV-F vaccine virus replicated to higher levels in the upper respiratory tract compared to CPI-RSV-F, but it replicated to lower levels in the lower respiratory tract compared to CPI-RSV-F.

Both CPI-RSV-F and W3AΔSH-RSV-F vaccines were highly efficacious at protecting the lung of cotton rats, inducing near sterilizing immunity at all vaccine doses tested, as indicated by the plaque assay and qPCR. All doses of both vaccines also induced statistically significant protection of the nose that was stronger for W3AΔSH-RSV-F compared to CPI-RSV-F immunization.

W3AΔSH-RSV-F reduced nasal viral load to almost undetectable levels at all three vaccine doses tested. For CPI-RSV-F, the 10⁶ PFU dose induced the strongest protection, followed by the 10⁵ PFU and 10⁴ PFU doses. Both CPI-RSV-F and control vaccines induced a strong NA response, with control, in all three doses tested, inducing higher titers of NA compared to CPI-RSV-F administered at the highest dose (10⁶ PFU). Lower doses of CPI-RSV-F induced a weaker NA response. Both CPI-RSV-F and W3A ΔSH—RSV-F immunizations induced high levels of binding IgG that was slightly higher for W3AΔSH-RSV-F vaccination. None of the vaccines induced the level of pulmonary histopathology or IL-4 mRNA expression that was seen in F1-RSV-immunized animals, with the exception of one animals immunized with the intermediate dose (10, PFU) of W3AΔSH—RSV-F. Overall, efficacy of W3AΔSH-RSV-F vaccine appears to exceed that of CPI-RSV-F in regards to nasal protection and neutralizing antibody response in all doses tested. Increasing the dose of W3AΔSH-RSV-F from 10⁴ to 10⁶ PFU provided no apparent advantage in terms of improving vaccine efficacy.

ii. Pulmonary Compositions

One embodiment provides a formulation and method for treating SARS-CoV-2 in the pulmonary system by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and of administration dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

Particle size for the aerosolized drug used in the present examples was measured at about 1.0-5.0 μm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

An agent of the present disclosure may be administered at once or may be divided into a number of multiple doses to be administered at intervals of time. For example, agents of the invention may be administered repeatedly, e.g., at least 2, 3, 4, 5, 6, 7, 8, or more times, or may be administered by continuous infusion. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that any concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

In some aspects, any of the PIV5-based constructs and methods described in WO 2013/112690 and WO 2013/112720 (which is hereby incorporated by reference herein in its entirety) may be used in the present invention.

As used herein, the term “subject” represents an organism, including, for example, a mammal. A mammal includes, but is not limited to, a human, a non-human primate, and other non-human vertebrates. A subject may be an “individual,” “patient,” or “host.” Non-human vertebrates include livestock animals (such as, but not limited to, a cow, a horse, a goat, and a pig), a domestic pet or companion animal, such as, but not limited to, a dog or a cat, and laboratory animals. Non-human subjects also include non-human primates as well as rodents, such as, but not limited to, a rat or a mouse. Non-human subjects also include, without limitation, poultry, horses, cows, pigs, goats, dogs, cats, guinea pigs, hamsters, mink, and rabbits.

As used herein “in vitro” is in cell culture and “in vivo” is within the body of a subject. As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

### EXAMPLES

### Example 1: Immunogenicity of CPI-RSV-F in African Green Monkeys (AGM) (Non-GLP Study)

Objective: To compare inunuogenicity of CPI-RSV-F and W3AΔSH-RSV-F administered intranasally as a single dose in the African green monkey model.

### Methods

Four monkeys in each dose group were immunized at Day 0 with 10⁶ PFU intranasally of either CPI-RSV-F (Group 6) or control (Group 1).

### Results

The antibody responses to RSV F-protein and PIV5 vector at Day 28 were determined by ELISA assays (plates coated with recombinant RSV F protein, source Sino Biologicals: 11049-V088 LC120C 2910, or PIV5 wild type virus) as shown in Table 4. In summary, all animals developed antibodies against the F protein expressed by the CPI or W3AΔSH vector with comparable RSV F ELISA titers between the two groups.

TABLE 4
Serum antibody response to RSV F and PIV5 in the AGM study.

| | Animal | RSV F ELISA titer | | PIV5 ELISA titer | |
|---|---|---|---|---|---|
| Group | ID | Day 0 | Day 28 | Day 0 | Day 28 |
| Group 1 | #1 _09818 | 120 | 3240 | <40 | 3240 |
| W3AΔSH- | #2_09836 | 120 | 3240 | 40 | 3240 |
| RSV-F | #3_90071 | 40 | 1080 | 40 | 9720 |
| | #4_58181 | 40 | 1080 | 120 | 9720 |
| Group 6 | #1_09669 | 360 | 1080 | <40 | 1080 |
| CPI-RSV-F | #2_09839 | 360 | 1080 | 40 | 1080 |
| | #3_09854 | 40 | 3240 | <40 | 360 |
| | #4_09861 | 120 | 3240 | <40 | 9720 |

Cell Mediated Responses: RSV F protein-specific cellular responses were evaluated by intracellular cytokine staining (ICS) assay. Following immunization, blood was collected at days −1, 14, and 28 to quantify the RSV F protein-specific CD4 and CD8 cellular responses. INFγ, TNFα, MIP-1b, IL-13, and CD107a positive cells were quantitated (see FIGS. 1A-1B). PBMCs collected at day −1 prior to immunization failed to respond to RSV F peptide stimulation as expected. Following immunization, AGMs immunized with W3AΔSH-RSV-F (Group 1) and CPI-RSV-F (Group 6) generated CD4 and CD8 cellular responses that are RSV F protein-specific as assessed on Day 14 and Day 28. No F-specific cellular response was detected in the control animals (data not shown), and the cellular responses from animals immunized with W3AΔSH-RSV-F and CPI-RSV-F increased from day 14 to day 28 post-immunization (FIGS. 1A-1B). W3AΔSH-RSV-F elicited lower CD4 responses than CPI-RSV-F at day 14, but the difference was not statistically significant. At day 28 post-immunization. W3AΔSH-RSV-F induced higher CD4 responses than CPI-RSV—F, but again this difference was not statistically significant. The CD8 specific responses elicited by W3AΔSH-RSV-F were higher than CPI-RSV-F, however the difference was not statistically significant on day 14 (P=0.0883) or day 28.

Both CPI-RSV-F and W3AΔSH-RSV-F elicited immune responses against RSV F protein. The antibody responses against RSV F protein elicited by either CPI-RSV-F or W3AΔSH-RSV-F were comparable following a single intranasal dose at 10⁶ PFU. The CD4 and CD8 cellular responses elicited by CPI-RSV-F were slightly lower than W3AΔSH-RSV-F, but the difference was not statistically significant.

### Example 2: Manufacturing Process and Process Controls

### Step 1: Vaccine Vector Construction

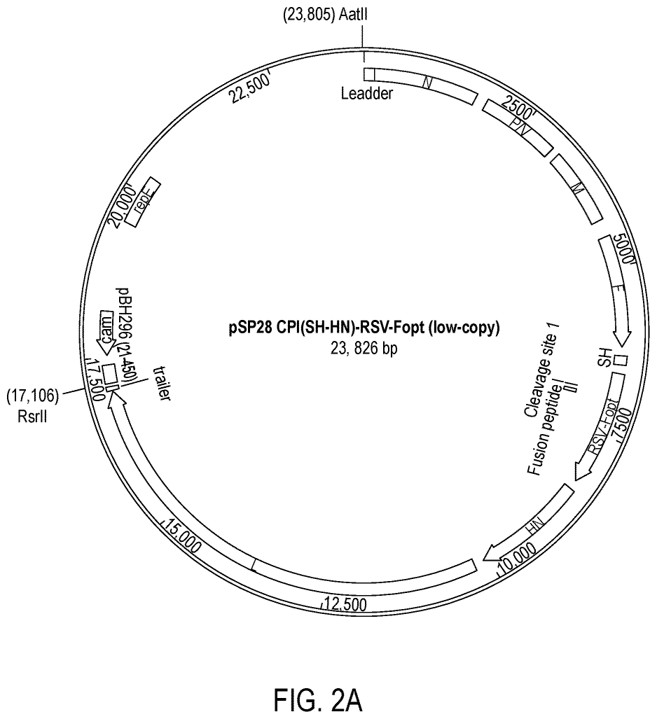

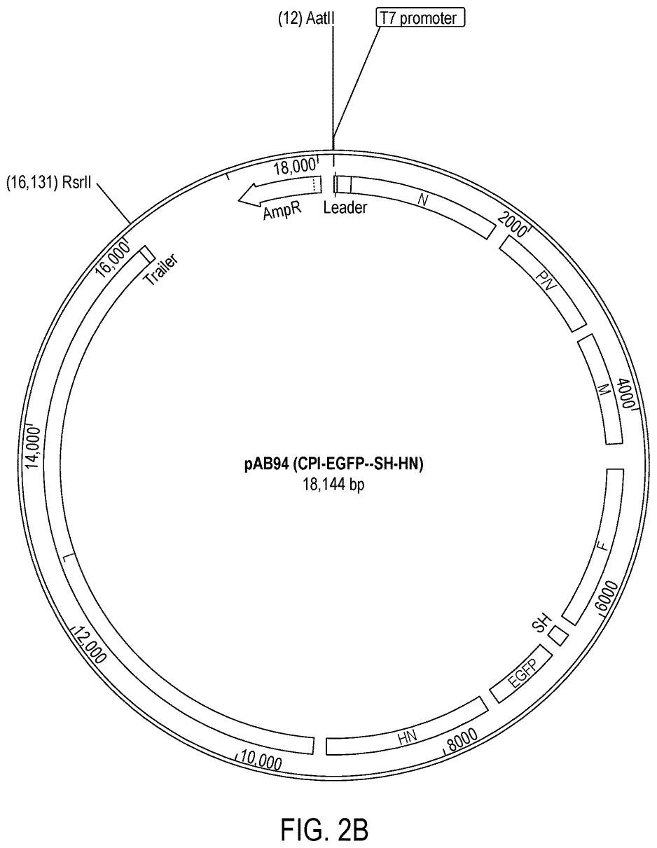

Construction of pCVL41 (CPI-RSV-F) antigenomic cDNA: CPI-RSV-Fopt genome was obtained through RsrII and AatII restriction enzyme digestion of pSP28 plasmid, pSB28 is a low copy plasmid containing the codon optimized RSV F protein gene (Fopt, sequence based on GenBank Accession M74568, RSV A2 strain, codon optimized for humans) inserted between the SH and HN junction of the CPI antigenomic cDNA (FIG. 2A) (3). pAB94 plasmid with the EGFP gene inserted between the SH and HN junction of the CPI antigenomic cDNA is a high copy plasmid (FIG. 2B). The RsrII and AatII restriction enzyme digested Fopt gene from CPI-RSV-Fopt was inserted into the RSrII and AatII digested pAB94 to replace the EGFP gene with the Fopt using T4 DNA ligase.

The ligated cDNA was transformed into TOP10 competent cells, single colonies were grown in LB media containing chloramphenicol.

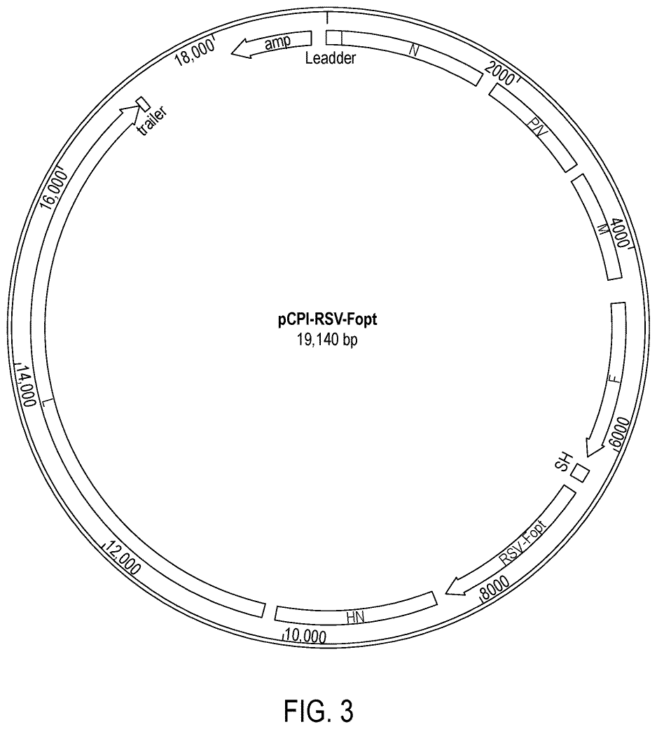

The resultant plasmid DNA (designated pCVL41 or pCPI-RSV-Fopt; FIG. 3) was purified by Qiagen miniprep kit and sequenced. The codon optimized RSV-F gene inserted between the SH and HN gene junction was confirmed to be correct by Sanger sequencing.

### Step 2: Vaccine Vector Rescue

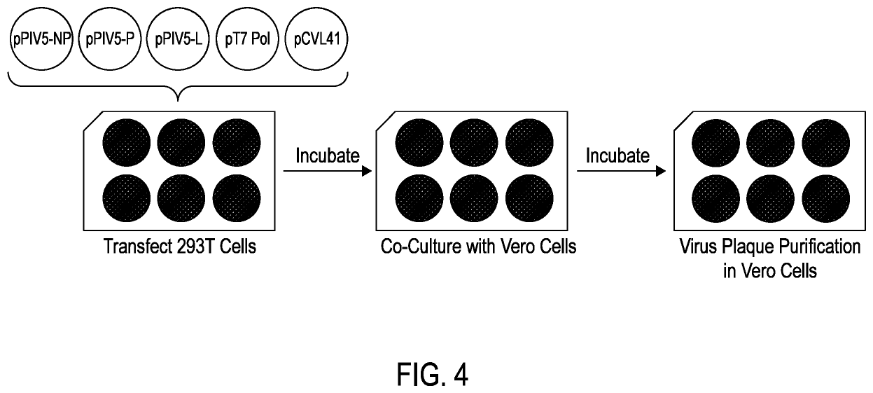

Rescue of the recombinant vector virus used in the production of CPI-RSV-F (CPI-RSV-F) was performed. For the rescue of the recombinant CPI-RSV-F virus, the pCPI-RSV-Fopt plasmid was transfected into serum-free 293T suspension cells(obtained from GenHunter Corporation) together with plasmids encoding the PIV5 NP, P, L proteins and T7 RNA Polymerase allowing for the rescue of recombinant virus from the transfected cell culture (FIG. 4).

The supporting PIV5 plasmid clones encoding the nucleoprotein (N), phosphoprotein (P) or large polymerase protein (L) were described previously (1,2) each gene is under the T7 promoter in pCAGGSvector. The following 5 plasmids were used in the generation of CPI-RSV-F RVS.
1. pCPI-RSV-Fopt (pCVL41-CPI-RSV-F antigenomic cDNA): This plasmid is a high-copy plasmid and it contains an ampicillin resistance gene. It encodes RSV F antigen gene inserted between the SH and HN gene junction of canine parainfluenza virus (CPI).
2. pCAGGS-NP Plasmid: This plasmid contains the PIV5 NP gene under the T7 promoter with anampicillin resistance gene.
3. pCAGGS-P Plasmid: This plasmid contains the PIV5 P gene under the T7 promoter with anampicillin resistance gene.
4. pCAGGS-L Plasmid: This plasmid contains the PIV5 L gene under the T7 promoter with anampicillin resistance gene.
5. pCAGGS-T7 Plasmid (same as pBH437-T7): This plasmid encodes the T7 RNA polymerase gene under the SV40 promoter.

The medium used for virus rescue was CDM4HEK293 medium (Hyclone) with 4 mM GlutaMAX (Gibco).

Following two days of incubation, the 293T cells were co-cultured with serum-free Vero cells (P159) passaged from Vero MCB cells (obtained from CRL: African Green Monkey Kidney (WHO Vero 10-87) CyanVac MCB DOM:19AUG2020-P148). After 4 days of incubation, 2 mL of supernatant containing the rescued virus was obtained and mixed with 10×SPG and stored at −80° C.

### Step 3: Plaque Purification and Expansion of Virus Rescue Seed

Aliquots of the frozen stock with rescued virus were serially diluted to perform a plaque assay on serum-free Vero cells (P155-derived from WHO Vero 10-87 CyanVac MCB DOM:9AUG2(20) in 6-well plates, with the objective of obtaining 6 isolated single plaques. A 1000 μL pipette tip was used to poke a single plaque and re-suspend in VP-SFM media containing 4 mM GlutaMAX. The re-suspended plaque was then used to infect fresh serum-free Vero cells (P155—derived from WHO Vero 10-87 CyanVac MCB DOM:19AUG2020) in a 6-well plate. After 6 days. the supernatant (2 mL) from the 6-well plate infected with single plaque was mixed with 10% 10×SPG and stored at −80° C. (to result in 1×SPG). This material was designated as Lot #CPI-RSV-F-PP1-PQ10-42621, date of manufacture Apr. 6, 2021). Part of the supernatant (140 μL was used to do RNA extraction and RT-PCR to verify viral genomic sequence. The RT-PCR was done with the primers described in Table 5.

TABLE 5
Primers used for amplifying viral cDNAs
for sequencing of CPI-RSV-F
antigenomic cDNA

| | | | SEQ |
|---|---|---|---|
| | | | ID |
| Number | Name | Sequence (5′ to 3′) | NO: |
| 1 | CVL1 | ACCAAGGGGAAAATGAAGTGGTGAC | 2 |
| 2 | CVL6 | GGAGAAGCTCAGATCAGTGGGATC | 3 |
| 3 | CVLS | ACAGTGGATTCCATAAGGTTCCTGC | 4 |
| 4 | CVL11 | CACTGACAGAAATGTCGGAATGAAGAG | 5 |
| 5 | CVL10 | TCCAGGTCACTGTTAGGAAGACATC | 6 |
| 6 | CVL20 | GCTCGATTACCTAGATTGACCGAGAC | 7 |
| 7 | CVL17 | CAAGATGCACCTTCTCTCCAGTG | 8 |
| 8 | CVL154 | ATCACACTCGAGCTGACATCTGTC | 9 |
| 9 | CVL152 | CGTGAGTACCTACATGCTGACA | 10 |
| 10 | CVL25 | CGGATTGATACGCTGAGTTGCTG | 11 |
| 11 | CVL19 | TAACTCTGCAGTCGCTCTACCTC | 12 |
| 12 | CVL27 | CAGTCTGAGTCATGCCATGCTG | 13 |
| 13 | CVL26 | GCATTCAGAATCTCAGCTATGGACTG | 14 |
| 14 | CVL33 | TCAATGATGATGTGAGCTACACGC | 15 |
| 15 | CVL32 | GCTCTGATGGAACTCAGTACGAATC | 16 |
| 16 | CVL37 | GOTTCTCTATTGAACTCAACCCTGAG | 17 |
| 17 | CVL36 | GTGACCGATCCATGAATCTCATTGC | 18 |
| 18 | CVL42 | ACCAAGGGGAAAACCAAGATTAATCC | 19 |

Step 4: Manufacture of prMVS (Lot #210519MCBCHD-PQ10) on Serum Free Vero Cells

An aliquot from Lot #CPI-RSV-F-PP1-PQ10-42621 (plaque purified virus) was used to infect serum-free Vero cells (P159, derived from WHO Vero 10-87 CyanVac MCB DOM:19AUG2020) in T75 flask, incubated at 37° C. for 5 days and the cell culture supernatants were centrifuged at 1,500 rpm for 10 minutes at 4° C. to remove cell debris. The clarified supernatants (20 mL) were mixed with 10% 10×SPG with 10% Arginine (Sigma Aldrich), fast frozen in 1 mL aliquots and stored at −80° C. as pre-MVS stock. The pre-MVS was designated as Lot #210519MCBCHD-PQ10. The pre-MVS was sequenced from the NP gene through the L gene to confirm the viral genomic sequence and proper insertion of the RSV F protein (a single silent mutation was found in AA54 of the NP).

The pre-MVS (Lot #210519MCBCHD-PQ10) prior to use in cGMP production of the MVS was tested at CRL for sterility (direct method), bacteriostasis and fungistasis, mycoplasma, mycobacteria, presence of porcine and bovine circoviruses, and in apparent viruses.

The ability of the CPI-RSV-F vaccine vector construct to infect Vero cells and express functional F-protein including prefusion F protein was assessed for both pre-MVS and MVS stock as detailed below using immunofluorescence assay (IFA). In addition, the pre-MVS and MVS were sequenced by Sanger method to confirm presence of correct F-protein gene insert in the PIV5 viral backbone (sequenced from the leader region to the trailer region).

The following characterization testing was performed on pre-MVS of Lot #210519MCBCHD-PQ10).

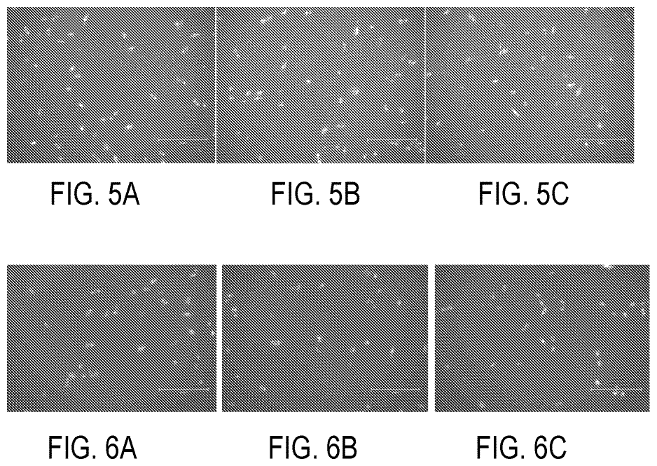

Expression of F-protein by CPI-RSV-F pre-MVS in infected Vero cells by IFA: The expression of F-protein by CPI-RSV-F was assessed by IFA staining for PIV5 vector (HN) and RSV F-protein using Palivizumab, a humanized murine monoclonal antibody that recognizes antigenic site 11 on both the prefusion (pre-F) and post fusion (post-F) conformations of the respiratory syncytial virus (RSV) F glycoprotein. The percentage F expression was determined by calculating number of cells staining positive for PIV5 (HN protein) that also stained positive for F-protein (FIGS. 5A-5C and Table 6).

TABLE 6
Percent Co-Expression CPI-RSV-F pre-MVS.

| Total Red Foci | Total Green Foci | |
|---|---|---|
| (PIV5) | (RSV F) | Percentage RSV F Expression |
| 43.4 | 43.4 | 100% |

Vero-SF cells were infected with CPI-RSV-F pre-MVS virus at dilutions 10-, 100- and 1000-fold. After 1-hour incubation at 37° C., the medium was changed, and cells were incubated for 18 hours at 37° C. Immunostaining was performed using mouse anti-PIV5-HN and human anti-F (Palivizumab) antibodies followed by anti-mouse Cy³ and anti-human FITC secondary antibodies, respectively. FIGS. 5A-5C are representative images of wells infected with CPI-RSV-F pre-MVS showing both red and green cells. Table 6 shows the percent expression of RSV F protein in PIV5-infected cells. Images were taken at 10×.

Expression of F-protein by CPI-RSV-F MVS infected cells by IFA: The expression of F-protein by CPI-RSV-F was assessed by IFA staining for PIV-5 vector (HN) and F-protein as per CVL-protocol-034. The percentage expression was determined by calculating number of cells staining positive for PIV5 (HN protein) that also stained positive for RSV F-protein (FIGS. 6A-6C and Table 7).

TABLE 7
Percent Co-expression of RSV-F and PIV5 for CPI-RSV-F (MVS).

| Total Red Foci | Total Green Foci | |
|---|---|---|
| (PIV5) | (RSV F) | Percentage RSV F Expression |
| 35.4 | 35.2 | 99.4% |

Vero-SF cells were infected with CPI-RSV-F MVS virus at dilutions 100-, 1000-, and 10000-fold. After 1-hour incubation at 37° C., media was changed, and cells were incubated for 18 hours at 37° C. Immunostaining was performed using mouse anti-PIV5-HN and human anti-F (Palivizumab) antibodies followed by anti-mouse Cy3 and anti-human FITC secondary antibodies, respectively. FIGS. 6A-6C are representative images of wells infected with CPI-RSV-F MVS showing both green and red cells. Table 7 shows the percent expression of RSV F protein in PIV5-infected cells. Images were taken at 10×.

### Step 5: Manufacturing Process for MVS

The production of the CPI-RSV-F MVS was performed in adherence to cGMP. The Master Virus Seed stock (MVS) which also serves as the Phase 1 clinical lot material was produced using serum free Vero cells. CyanVac MCB (DOM:19AUG2020, expanded at CRL to passage 152) in adherence to cGMP at CRL August 2021 (CBR-1862).

Vaccine bulk substance manufacture: The Vero cells grown in T225 flasks in serum free media (VP-SFM) were infected with CPI-RSV-F pre-MVS Lot #210519MCBCHD-PQ10 (using 2 vials) at MOI of 0.002 at 37° C. for 7 days. The cell culture fluid of infected cells constitutes the crude vaccine bulk substance (3.0 liter). Samples of the MVS crude bulk virus fluid were taken for testing of: Sterility (direct method), bacteriostasis and fungistasis, in vitro mycoplasma testing (agar cultivable and non-cultivable), tissue culture safety testing (GP-V810.2), in vitro mycobacterium testing, inapparent viruses. PBERT, potency.

The vaccine bulk substance was clarified by centrifugation at 1,500 rpm for 10 min at 2-8° C. resulting in 1.2 L followed by filtration through a 0.45 μM PES filter unit from Thermo Fisher resulting in 1.2 L. The clarified and filtered vaccine bulk substance is immediately formulated with 10×SPG to stabilize the virus prior to fill.There is no hold of the vaccine bulk substance before formulation and fill.

During the virus production process, control harvest fluid was prepared using uninfected Vero cells fromthe same production lot. The control fluids were stored at −60° C. or below. The production control fluid was harvested and tested for the Bacteriostasis and Fungistasis Bulk Product (Direct Method), Sterility, Detection and Quantitation of Residual Vero DNA and Determination of Endotoxin Levels (LAL).

Vaccine drug product manufacture (formulation and fill)/MVS: The filtered viruses were stabilized by formulation with 10% 10×SPG buffer and dispensed using a calibrated repeater pipette in 1 mL volumes into 2 mL cryovials to make the MVS stock/Vaccine Product (total 1281 vials, 1 mL per vial). The dispensed MVS/Vaccine product was flash frozen in dry ice/methanol bath and stored at −60° C. or below.

Clinical lot material: For the proposed initial Phase 1 trial, the Master Viral Seed (MVS), Lot #CPI-RSV-F (CPI-RSV-F-210519PQ10MVS DOM: 26AUG2021), is used as the vaccine drug product.

MVS characterization: In addition, genetic identity was confirmed by sequencing of viral antigenomic cDNA from the NP gene through the L gene region to confirm identity.

### Example 3: Comparative Studies Between PIV5 Vectored Control and CPI-RSV-F Vaccine Candidates in BALB/c Mice

### Materials and Methods

The objective of the two mice studies (CHD03 and CHD04) in this report was to compare the immunogenicity and protective efficacy of two RSV vaccine candidates: W3AΔSH-RSV-F and CPI-RSV-F, in the BALB/c mouse model. In addition, two other vaccine candidates: W3A-RSV-F (SH-HN), and CPIΔSH-RSV-F were included in the studies. Antibody titers were measured by RSV F specific ELISA assay and cellular immune responses were measure by ELISPOT assay. The protection of vaccinated mice against wild type RSV strain challenge infection in the lower respiratory tract was determined by plaque assay of lung homogenates (Table 8).

TABLE 8
Summary of W3AΔSH-RSV-F and CPI-RSV-F data in CHD03 and CHD04 studies.

| | | | | Anti-F antibody | | | | | |
|---|---|---|---|---|---|---|---|---|---|
| | | | | titer | | IFN-γ | | | |
| | | | | (reciprocal | | secreting cells | | | |
| | | | | dilution in | | (per 10⁶ | | RSV titer in lung | |
| | | Number | | Log₁₀/mL) | | splenocytes) | | (Log₁₀ (PFU/g) | |
| | Test | of mice | | 27 dpi | | 27 dpi | | 4 dpc | |
| Group | Articles | CHD03 | CHD04 | CHD03 | CHD04 | CHD03 | CHD04 | CHD03 | CHD04 |
| A-C | PBS | 15 | 15 | 1.3 | 1.3 | 3 | 0 | 3.21 | 3.33 |
| | (Negative | | | | | | | | |
| | Control) | | | | | | | | |
| D-F | RSV_rA2 | 15 | 15 | 2.9 | 3.3 | 10 | 10 | 1.39 | 1.32 |
| | (Positive | | | | | | | | |
| | Control) | | | | | | | | |
| J-L | W3AΔSH- | 15 | 15 | 3.1 | 3.4 | 28 | 35 | 1.35 | 1.37 |
| | RSV-F; | | | | | | | | |
| | 10⁵ PFU. | | | | | | | | |
| M-O | CPI-RSV-F; | 15 | 15 | 3.0 | 3.2 | 23 | 48 | 1.48 | 1.40 |
| | 10⁵ PFU. | | | | | | | | |

dpi = days post immunization; dpc = days post challenge

The critical materials and reagents used are listed in Table 9. The viruses used in the experiment are listed in Table 10.

TABLE 9
Critical Materials and Reagents

| Item | Source |
|---|---|
| Tissue homogenate samples | Biao He Lab, University of |
| | Georgia |
| Vero cells | ATCC, Cat#CCL-81 |
| Human anti-RSV-F mAb (Palivizumab) | MedImmune/AstraZeneca |
| Goat anti-human IgG H&L-HRP | Novex, Cat#A18811 |
| AEC Peroxide Substrate Kit | Vector, Cat#SK-4200 |
| RSV F protein | SinoBiological, 11049-V08B |
| | lot LC120C2910 |
| RSV F Peptide (85-93) | Lot#150909SP |
| ELISPOT Mouse IFN-γ Set | BD, Cat#551083 |
| ELISPOT AEC Substrate Set | BD, Cat#551951 |

TABLE 10
Viruses

| Item | Lot | Titer (PFU/mL) | Source |
|---|---|---|---|
| RSV_rA2 strain | 190204AB | 7.6 × 10⁶ | Biao He Lab, UGA |
| control | 181204CHD | 2.3 × 10⁷ | Biao He Lab, UGA |
| control | 181204CHD | 1.9 × 10⁷ | Biao He Lab, UGA |
| CPI-RSV-F | 170501SP | 2.1 × 10⁶ | Biao He Lab, UGA |
| CPIΔSH-RSV-F | 170501SP | 1.7 × 10⁷ | Biao He Lab, UGA |

All viruses were diluted down to 2×10⁶ PFU/mL with 1×PBS prior to vaccination. RSV_rA2 stock was not diluted prior to challenge.

Experimental groups applicable to the ELISA, ELISPOT, and plaque assay tests are summarized in Table 11 and 12.

TABLE 11
Dosing groups and testing CHD03.

| | | | | ELISPOT | | |
|---|---|---|---|---|---|---|
| | | Number | Dosing | ELISA | Challenge | Necropsy |
| Group | Dose Level | of Mice | Day | Day | Day | Day |
| A-C | Negative | 15 | Day 0 | Day 27 | Day 35 | Day 39 |
| | Control: | | | | | |
| | PBS | | | | | |
| D-F | Positive | 15 | Day 0 | Day 27 | Day 35 | Day 39 |
| | control: | | | | | |
| | RSV_rA2; | | | | | |
| | 10⁵ PFU | | | | | |
| G-I | W3A-RSV-F; | 15 | Day 0 | Day 27 | Day 35 | Day 39 |
| | 10⁵ PFU | | | | | |
| J-L | W3AΔSH- | 15 | Day 0 | Day 27 | Day 35 | Day 39 |
| | RSV-F; | | | | | |
| | 10⁵ PFU | | | | | |
| M-O | CPI-RSV-F; | 15 | Day 0 | Day 27 | Day 35 | Day 39 |
| | 10⁵ PFU | | | | | |
| P-R | CPIΔSH- | 15 | Day 0 | Day 27 | Day 35 | Day 39 |
| | RSV-F; | | | | | |
| | 10⁵ PFU | | | | | |

TABLE 12
Dosing groups and testing CHD04.

| | | | | ELISPOT | | |
|---|---|---|---|---|---|---|
| | | Number | Dosing | ELISA | Challenge | Necropsy |
| Group | Dose Level | of Mice | Day | Day | Day | Day |
| A-C | Negative | 15 | Day 0 | Day 27 | Day 35 | Day 39 |
| | Control: | | | | | |
| | PBS | | | | | |
| D-F | Positive | 15 | Day 0 | Day 27 | Day 35 | Day 39 |
| | control: | | | | | |
| | RSV_rA2; | | | | | |
| | 10⁵ PFU | | | | | |
| G-I | W3A-RSV-F; | 15 | Day 0 | Day 27 | Day 35 | Day 39 |
| | 10⁵ PFU | | | | | |
| J-L | W3AΔSH- | 15 | Day 0 | Day 27 | Day 35 | Day 39 |
| | RSV-F; | | | | | |
| | 10⁵ PFU | | | | | |
| M-O | CPI-RSV-F; | 15 | Day 0 | Day 27 | Day 35 | Day 39 |
| | 10⁵ PFU | | | | | |
| P-R | CPIΔSH- | 15 | Day 0 | Day 27 | Day 35 | Day 39 |
| | RSV-F; | | | | | |
| | 10⁵ PFU | | | | | |

Immunization of mice: Six-to-eight week old female BALB/c mice were anesthetized by intraperitoneal injection of 250 μL of 2, 2, 2-tribromoethanol in tert-amyl alcohol (Avertin). Immunization was performed by intranasal administration of 10⁵ PFU of each vaccine candidate plus RSV_rA2 positive control in a 50 μL (25 μL per nostril) volume. Negative controls were treated intranasally with 50 μL (25 μL per nostril) of PBS. The RSV_rA2 positive control was used to mimic a natural RSV infection prior to vaccination. All animal experiments were performed according to the protocols approved by the Institutional Animal Care and Use Committee at the University of Georgia.

Twenty-seven days (mice in Groups A, D, G, J, M, P for CHD03 and Groups A, E, G, K, M, P for CHD04) or 28 days (Groups B, C, E, F, H, I, K, L, N, O, Q, R for CHD03 and Groups B, C, D, F, H, I, J, L, N, O, Q, R for CHD04) post-immunization, blood was collected via cheek bleed for serological analysis. Spleens from mice in cages A. D. G. J. M. P for CHD03 and cages A, E, G, K, M, P for CHD04 were also collected 27 days post-immunization to perform ELISPOT assays. Thirty-five days post-immunization, mice were anesthetized by intraperitoneal injection of 250 μL of Avertin and Groups B, C, E, F, H, I, K, L, N, O, Q, R for CHD03 and Groups B, C, D, F, H, I, J, L, N, O, Q, R for CHD04 were challenged intranasally with 3.8×10⁵ PFU of RSV A2 in a 50 μL (25 μL per nostril) volume. After challenge, mice were continued to be house together per their vaccination group. Four days later, lungs were collected from 7-10 mice per group to assess viral burden by performing plaque assays on lung homogenates. Experimental timelines are shown in FIGS. 7 and 8.

Anti-F ELISA Assay: Anti-F antibody titer was determined by anti-F ELISA assay per CVL SOP-049. The Immulon 2HB 96 well plates were coated with 50 μL/well of F protein of RSV at 12.5 ng/mL overnight. The serum samples were prediluted by 10-fold followed by 3-fold serial dilution and added to F protein-coated plate (50 μL/well) at room temperature for 1 hour. After plate wash, 50 μL/well goat anti-mouse IgG HRP conjugated antibody at 1:1250 dilution was added and incubated at room temperature for 1 hour. Following wash, 100 μL/well of SureBlue Reserve TMB Microwell Peroxidase Substrate was added for 3-5 min and the reaction was stopped by adding 100 μL/well of IN HCl. The plates were read with plate reader (SpectraMax iD3 Multi-mode Microplate Reader) at 450 nm immediately after adding HCL. Samples that exceed OD₄₅₀ cutoff value of 0.20 are considered positive in at least two consecutive dilutions. Antibody titer is defined as the highest reciprocal dilution with positive signal. This assay was performed at Blue Lake Biotechnology in Los Gatos, CA.

Enzyme-Linked ImmunoSpot Assay (ELISPOT): Levels of IFN-γ secreting cells were measured by ELISPOT assay using BD™ ELISPOT Mouse IFN-γ Set. BD™ ELISPOT plates were coated with purified anti-mouse IFN-γ antibody 24 hours prior to performing assay. Mouse spleens (n=5, per group) were collected 27 days post-immunization and put into 15 mL conical tubes containing 5 mL of HBSS. Splenocytes were prepared by pressing spleens through a 70 μM cell strainer, incubating them with ACK lysis buffer, washing them with HBSS, and re-suspending in complete tumor medium (CTM) to a concentration of 5×10⁶ cells/mL. The capture antibody solution was removed from the plates and then the plates were washed 5-6 times with PBS. The plates were then blocked with CTM for 90 min. The blocking solution was discarded, and 0.1 μg of RSV F peptide (85-93) in 50 μL of CTM were added to the wells. Next, 50 μL of splenocytes (2.5×10⁵ cells/well) were added to plates and incubated at 37° C. 5% CO₂, for 48 h. The spots were immunostained according to the BD™ ELISPOT Set instruction manual and counted using an ImmunoSpot@analyzer (Cell Technology Limited, CTL). Results were presented as mean number of IFN-γ secreting cells per 10⁶ splenocytes. This assay was performed at the University of Georgia in Athens, GA.

Plaque Assay: RSV viral titer in lung homogenate was performed in Vero cells by plaque assay. Briefly, mouse lungs were collected in gentleMACS M tubes containing 3 mL of Opti-MEM with 1% BSA and kept on ice. The lungs were weighted and then homogenized using the Protein_01 program of a gentleMACS Dissociator at 4° C. and then centrifuged for 10 min at 3000×g. Supernatant was used to perform 3-fold serial dilutions at a total volume of 0.6 mL from undiluted to 1:27. Vero cells in a 24-well plate were infected with 100 μL of each dilution in triplicate. After 1 hour adsorption at 3TC, the inoculum was removed, plates were washed 1× with PBS, and approximately 1 mL of methyl cellulose was added to each well. After a 7-day incubation, the methyl cellulose was removed, and cells were fixed with approximately 500 μL of 60% Acetone/40% Methanol for 20 minutes. Cell were washed and then blocked with 400 μL/well of blotto for 30 minutes. After blocking, cells were incubated for 1 hour at room temperature with 200 μL/well of human anti-RSV-F antibody (Palivizumab, 1 mg/mL) diluted 1:1000. After plate wash, 200 μL/well goat anti-human IgG HRP conjugated antibody at 1:1000 dilution was added and incubated at room temperature for 1 hour. Following wash, 200 μL/well of AEC substrate was added for 30 minutes at room temperature. Viral titer was determined by counting plaques at the dilution where range of plaque counting was between 10-100. Results were reported as PFU/g of lung. This assay was performed at the University of Georgia in Athens, GA.

### Results

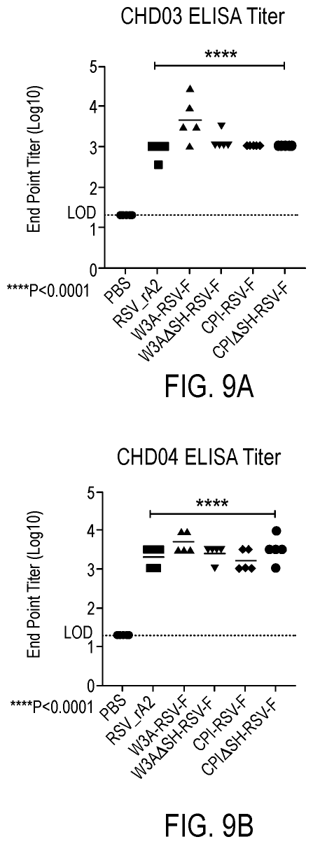

Anti-F antibody titer: Anti-F antibody titers were obtained from animals in Groups A, D, G, J, M, P for CHD03 and Groups A, E, G, K, M, P for CHD04 at Day 27 post vaccination and results are shown in FIG. 9A-9B. Overall, anti-F antibody titers were significantly higher in all vaccinated groups compared to the PBS control group. Anti-F antibody titers in Group J (W3AΔSH-RSV-F) and Group M (CPI-RSV-F) in CHD03 and Group K (W3AΔSH-RSV-F) and Group M (CPI-RSV-F) in CHD04, were very similar. In CHD03, W3AΔSH-RSV-F had a geometric mean titer of 3.1 Log₁₀/mL while CPI-RSV-F had a geometric mean titer of 3.0 Log₁₀/mL. In CHD04, W3AΔSH-RSV-F had a geometric mean titer of 3.4 Log₁₀/mL, while CPI-RSV-F had a geometric mean titer of 3.2 Log₁₀/mL. W3A-RSV-F in both CHD03 and CHD04 had the highest anti-F antibody titer with a geometric mean titer of 3.7 Log₁₀/mL in both experiments. W3AΔSH-RSV-F and CPI-RSV-F groups had similar anti-F antibody titers to RSV_rA2 positive control group in both CHD03 and CHD04. CPIΔSH-RSV-F also elicited comparable serum antibody levels in both studies.

These results indicate that both W3AΔSH-RSV-F and CPI-RSV-F as well as other two vaccine candidates elicit similar levels of anti-F antibodies in BALB/c mice at the dose of 1×10⁵ PFU administered intranasally.

F-specific cellular immune responses: Cellular immune responses induced by the vaccine candidates were measured by levels of IFN-γ secreting cells. In CHD03, Group J (W3AΔSH-RSV-F) and Group M (CPI-RSV-F) had higher levels of IFN-γ secreting cells, with Group J having a geometric mean of 28 IFN-γ secreting cells per 10⁶ splenocytes and Group M having a geometric mean of 23 IFN-γ secreting cells per 10⁶ splenocytes. These values were not statistically significant from the PBS group, but they were also not statistically significant from each other. Group G (W3A-RSV-F) had the highest value compared to the PBS group, with a geometric mean of 57 IFN-γ secreting cells per 10⁶ splenocytes, which was not significantly different from the other groups. Values from W3AΔSH-RSV-F and CPI-RSV-F groups are similar to the levels of IFN-γ secreting cells seen in RSV_rA2 group.

In CHD04, Group K (W3AΔSH-RSV-F) and Group M (CPI-RSV-F) had the highest levels of IFN-γ secreting cells, with Group K having a geometric mean of 35 IFN-γ secreting cells per 10⁶ splenocytes and Group M of 48 cells per 10⁶ splenocytes. These values are statistically significant from the PBS control group, but they were not statistically significant from each other. Out of both vaccine groups of interest, only CPI-RSV-F group had a significantly higher amount of IFN-γ secreting cells than RSV_rA2 positive control group. W3A-RSV-F and CPIΔSH-RSV-F also elicited F specific cellular responses.

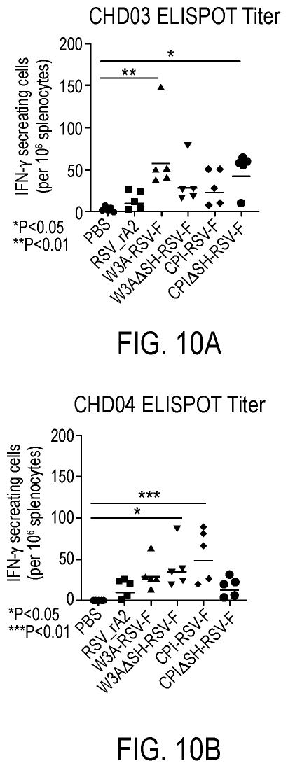

FIGS. 10A-10B indicate that both W3AΔSH-RSV-F and CPI-RSV-F vaccine candidates of interest as well as the other two vaccine candidates (W3A-RSV-F and CPIASH—RSV-F) elicit similar levels of cell-mediated immune responses in BALB/c mice.

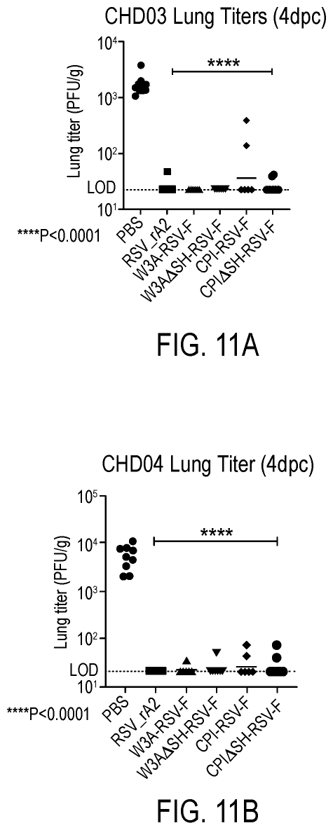

RSV challenge virus lung titers: The four RSV vaccine candidates were evaluated for their protection against RSV_rA2 virus challenge infection in the lower respiratory tract of mice. Overall, all vaccine groups had significantly lower lung viral titers compared to the PBS control in both CHD03 (FIG. 11A) and CHD04 (FIG. 11B), which had geometric mean titers of 3.21 Logic PFU/g and 3.33 Logic PFU/g respectively. In CHD03, RSV challenge virus titers in Groups K and L (W3AΔSH-RSV-F), was 1.35 Log₁₀ PFU/g, while Groups N and O (CPI-RSV-F) had a geometric mean titer of 1.48 Log₁₀ PFU/g. These values are not statistically significant from each other. The other vaccine candidates tested (W3A-RSV-F and CPIΔSH-RSV-F) had geometric mean titers of 1.35 Log₁₀ PFU/g and 1.40 Log₁₀ PFU/g, respectively. These values are also significantly lower than the PBS control. In CHD04, Groups J and L (W3AΔSH-RSV-F) had a geometric mean titer of 1.37 Log₁₀ PFU/g, while Groups N and O (CPI-RSV-F) had a geometric mean titer of 1.40 Log₁₀ PFU/g. Similar to CHD03, these values are not statistically significant from each other, but are significantly lower than the PBS control group. The W3A-RSV-F and CPIΔSH-RSV-F vaccine candidates had geometric mean titers of 1.35 Log₁₀ PFU/g and 1.40 Log₁₀ PFU/g, respectively. These values are the same as experiment CHD03 and are also significantly lower than the PBS control group. Lung RSV viral titers in all vaccine groups were similar to that seen in RSV_rA2 positive control group in both CHD03 and CHD04.

FIGS. 11A-11B indicate that both W3AΔSH-RSV-F and CPI-RSV-F vaccine candidates as well as the other two vaccine candidates protected against RSV challenge virus infection in the lower respiratory tract of BALB/c mice.

### Example 4: Virus Replication in the Upper and Lower Respiratory Tracts of Cotton Rats

### Methods and Materials

Animals: Fifty five (55) inbred, 6-8 weeks-old, Sigmodon hispidus female and male cotton rats (source: Sigmovir Biosystems, Inc. Rockville MD) were maintained and handled under veterinary supervision in accordance with the National Institutes of Health guidelines and Sigmovir Institutional Animal Care and Use Committee's approved animal study protocol (IACUC Protocol #15). Each group of animals included 3 females (the first three animals in each group) and 2 males (the last 2 animals in each group). Cotton rats were housed in clear polycarbonate cages and provided with standard rodent chow (Harlan #7004) and tap water ad lib.

Virus: Respiratory Syncytial Virus strain A/A2 (RSV A/A2) (ATCC, Manassas, VA) was propagated in HEp-2 cells after serial plaque-purification to reduce defective-interfering particles. A pool of virus designated as hRSV A/A2 Lot #092215 SSM containing approximately 3.0×10⁸ pfu/mL in sucrose stabilizing media was used in this in vivo experiment. This stock of virus is stored at −80° C. and has been characterized in vivo in the cotton rat model and validated for upper and lower respiratory tract replication.

Procedure(s): Divide 55 female and male young adult cotton rats (6-8 weeks of age) into 9 groups of 5 animals each (3 females, 2 males), one group of 4 animals (2 females, 2 males), and 2 groups of 3 animals each (2 females and 1 male). Prebleed for serum collection and ear-tag all animals. Immunize or infect animals with 0.1 ml of preparation as indicated in Table 13 below.

TABLE 13
Immunization or Infection of Animals.

| | | | | | | | Day of |
|---|---|---|---|---|---|---|---|
| | | | | | | | harvest |
| | | | TA Dose | Immunization | | Challenge | (post-RSV |
| Group | N | Treatment | (per rat) | days | Route | (pfu/rat) | challenge) |
| A | 4 | PBS (Mock) | n/a | 0.28 | IM | PBS | 5 |
| B | 5 | PBS (Mock) | n/a | 0.28 | IN | RSV 5.0 | 5 |
| | | | | | | Log10 | |
| C | 5 | FI-RSV | 1:100 | 0.28 | IM | RSV 5.0 | 5 |
| | | Lot#100 | in PBS | | | Log10 | |
| D | 5 | RSV/A2 | 5.0 | 0 only | IN | RSV 5.0 | 5 |
| | | Live | Log10 | | | Log10 | |
| E | 5 | CPI-RSV-F | 10⁴ | 0 only | IN | RSV 5.0 | 5 |
| | | | PFU | | | Log10 | |
| F | 5 | CPI-RSV-F | 10⁵ | 0 only | IN | RSV 5.0 | 5 |
| | | | PFU | | | Log10 | |
| G | 5 | CPI-RSV-F | 10⁶ | 0 only | IN | RSV 5.0 | 5 |
| | | | PFU | | | Log10 | |
| H | 5 | W3AΔSH- | 10⁴ | 0 only | IN | RSV 5.0 | 5 |
| | | RSV-F | PFU | | | Log10 | |
| I | 5 | W3AΔSH- | 10⁵ | 0 only | IN | RSV 5.0 | 5 |
| | | RSV-F | PFU | | | Log10 | |
| J | 5 | W3AΔSH- | 10⁶ | 0 only | IN | RSV 5.0 | 5 |
| | | RSV-F | PFU | | | Log10 | |
| K | 3 | CPI-RSV-F | 10⁶ | 0 only | IN | Harvest on | |
| | | | PFU | | | day 4 post- | |
| L | 3 | W3AΔSH- | 10⁶ | 0 only | IN | PIV dosing | |
| | | RSV-F | PFU | | | | |

Sacrifice animals in groups K and L. Harvest the nasal tissue and homogenize in 3 ml of HBSS supplemented with 10% SPG for viral titrations. Harvest the lung en bloc and tri-sect for viral titration (left section, homogenize in 3 ml of HBSS supplemented with 10% SPG), histopathology (right section, inflate with 10% neutral buffered formalin), and qPCR (lingular lobe, flash frozen in liquid nitrogen).

Eyebleed all animals for serum collection. Boost animals in groups A, B, and C with 0.1 ml of preparation as indicated in the table above.

Eyebleed all animals for serum collection. Mock challenge intranasally (IN) group A with 0.1 ml of PBS (pH 7.4). Challenge in animals in groups B thru J with 0.1 ml of RSV/A2 (Lot #092215 SSM) at 10⁵ PFU per animal. Perform back titration on the challenge virus to confirm challenge dose.

Sacrifice all animals. Harvest the nasal tissue and homogenize in 3 ml of HBSS supplemented with 10% SPG for viral titrations. Harvest the lung en bloc and tri-sect for viral titration (left section, homogenize in 3 ml of HBSS supplemented with 10% SPG), histopathology (right section, inflate with 10% neutral buffered formalin), and qPCR (lingular lobe, flash frozen in liquid nitrogen). The animal sacrifice groups are shown in Table 14 and sample collections are shown in Table 15.

TABLE 14
Animal Sacrifice Groups.

| Groups | Animal #s |
|---|---|
| A | 124831-124834 |
| B | 124835-124839 |
| C | 124840-124844 |
| D | 124845-124849 |
| E | 124850-124854 |
| F | 124855-124859 |
| G | 124860-124864 |
| H | 124865-124869 |
| I | 124870-124874 |
| J | 124875-124879 |
| K | 124880-124882 |
| L | 124883-124885 |

TABLE 15
Sample Collections.

| Sample Collections | Day(s) of collection | | # of Sets |
|---|---|---|---|
| Lung and nose homogenates | 4 | (GrK-L) | 3* |
| Lung and nose homogenates | 54 | (GrA-J) | 2 |

| | | |
|---|---|---|
| Lung histology | 4 (GrK-L); 54 (GrA-J) | 1 |
| Serum | 0, 28, and 49 | 2 |
| Lung total RNA | 4 (GrK-L); 54 (GrA-J) | 1 |

Table 16 shows the endpoint assays protocol outline.

TABLE 16
Endpoint Assays Protocol

| Endpoint Assays | Samples | Storage |
|---|---|---|
| RSV viral titrations (GrA-J) | Lung homogenates | −80° C. |
| | Nose homogenates | |
| PIV viral titrations (GrK-L) | Lung homogenates | −80° C. |
| | Nose homogenates | |
| Pulmonary Histopathology (GrA-K) | H&E lung slides | RT |
| RSV Neutralizing Ab (GrA-J) | Serum | −20° C. |
| (RSV/A2) (d 0, 28, and 49) | | |
| RSV/A2 serum IgG ELISA (GrA-J) | Serum | −20° C. |
| (d 0, 28, 49) (4-dilution format) | | |
| qPCR (GrA-J) | Lung RNA | −80° C. |
| RSV/A2 NS-1 | | |
| gene; IL-4, IL-2, | | |
| IFN-γ | | |
| qPCR PIV (GrK, L) | Lung RNA | −80° C. |

Lung and nose RSV viral titration: Lung and nose homogenates are clarified by centrifugation and diluted in EMEM. Confluent HEp-2 monolayers are infected in duplicates with diluted homogenates in 24 well plates. After one hour incubation at 37° C. in a 5% CO₂ incubator, the wells are overlayed with 0.75% Methylcellulose medium. After 4 days of incubation, the overlay is removed and the cells are fixed with 0.1% crystal violet stain for one hour and then rinsed and air dried. Plaques are counted and virus titer is expressed as plaque forming units per gram of tissue. Viral titers are calculated as geometric mean±standard error for all animals in a group at a given time.

Pulmonary histopathology: Lungs are dissected and inflated with 10% neutral buffered formalin to their normal volume, and then immersed in the same fixative solution. Following fixation, the lungs are embedded in paraffin, sectioned and stained with hematoxylin and eosin (H&E). Four parameters of pulmonary inflammation are evaluated: peribronchiolitis (inflammatory cell infiltration around the bronchioles), perivasculitis (inflammatory cell infiltration around the small blood vessels), interstitial pneumonia (inflammatory cell infiltration and thickening of alveolar walls), and alveolitis (cells within the alveolar spaces). Slides are scored blind on a 0-4 severity scale. The scores are subsequently converted to a 0-100% histopathology scale.

RSV neutralizing antibody assay (60% reduction) for preclinical studies: Heat inactivated sera samples are diluted 1:10 with EMEM and serially diluted further 1:4. Diluted sera samples are incubated with RSV (25-50 PFU) for 1 hour at room temperature and inoculated in duplicates onto confluent HEp-2 monolayers in 24 well plates. After one hour incubation at 37° C. in a 5% CO2 incubator, the wells are overlayed with 0.75% Methylcellulose medium. After 4 days of incubation (6 days for RSV B), the overlay is removed and the cells are fixed with 0.1% crystal violet stain for one hour and then rinsed and air dried. The corresponding reciprocal neutralizing antibody titers are determined at the 60% reduction end-point of the virus control using a statistics program. The geometric means±standard error for all animals in a group at a given time are calculated.

RSV IgG ELISA for preclinical studies: Whole RSV inactivated with UV or purified F protein extracted from RSV-infected HEp-2 cells are diluted and coated onto 96 well ELISA plate overnight. The coating antigen is decanted and the plate is incubated in blocking solution for one hour at room temperature and subsequently washed. Diluted sera (1:500 in duplicates) along with the positive and negative controls are added to the wells and incubated at room temperature for one hour. After washing the plate, Rabbit anti Cotton Rat IgG (1:500) is added to all the wells and incubated for one hour at room temperature. This is followed by the incubation with Goat anti Rabbit IgG-HRP (1:6,000) for one hour at room temperature. At the end. TMB substrate is added to all the wells and incubated at room temperature for 15 minutes. TMB-Stop solution is added to all the wells and optical density at 450 nm is recorded. Geometric mean of the optical density (OD₄₅₀) is measured for all triplicate sera samples+standard error for all samples in a group per time-point.

Real-time PCRforpreclinical studies: Total RNA is extracted from homogenized tissue or cells using the RNeasy purification kit (QIAGEN). One μg of total RNA is used to prepare cDNA using Super Script II RT (Invitrogen) and oligo dT primer (1 μl, Invitrogen). For the real-time PCR reactions the Bio-Rad iQ™ SYBR Green Supermix is used in a final volume of 25 μl, with final primer concentrations of 0.5 μM. Reactions are set up in duplicates in 96-well trays. Amplifications are performed on a Bio-Rad iCycler for 1 cycle of 95° C. for 3 min, followed by 40 cycles of 95° C. for 10 seconds (s), 60° C. for 10 s, and 72° C. for 15 s. The baseline cycles and cycle threshold (Ct) are calculated by the iQ5 software in the PCR Base Line Subtracted Curve Fit mode. Relative quantitation of DNA is applied to all samples. The standard curves are developed using serially diluted cDNA sample most enriched in the transcript of interest (e.g., lungs from 6 hours post RSV infection of FI-RSV-immunized animals or PIV5-specific control). The Ct values are plotted against log₁₀ cDNA dilution factor. These curves are used to convert the Ct values obtained for different samples to relative expression units. These relative expression units are then normalized to the level of β-actin mRNA (“housekeeping gene”) expressed in the corresponding sample. For animal studies. mRNA levels are expressed as the geometric mean±SEM for all animals in a group at a given time.

### Results

Confirmation of the replication of vaccine viruses in the cotton rat model: To confirm replication of the PIV5-based vaccines in the respiratory tract of cotton rats, groups of 3 animals were inoculated with 10⁶ PFU of CPI-RSV-F or W3AΔSH-RSV-F intranasally and sacrificed four days later, with samples submitted for evaluation to Blue Lake Biotechnology (BLB). BLB conducted plaque assay on submitted samples. In brief, it was found that both candidate vaccine viruses replicated in the upper and lower respiratory track of cotton rats (Table 17). In CPI-RSV-F vaccinated animals, the vaccine virus replicated similarly in the upper (nose) and lower respiratory tract (lung), 3.9 and 3.5 Log₁₀ PFU/mL, respectively. W3AΔSH-RSV-F replicated at a higher titer in the upper respiratory tract (5.0 Log₁₀ PFU/mL) compared to lower respiratory tract (2.2 Log₁₀ PFU/mL). W3AΔSH-RSV-F vaccine virus replicated to higher levels in the upper respiratory tract compared to CPI-RSV-F, but it replicated to lower levels in the lower respiratory tract compared to CPI-RSV-F. Overall, replication of both vaccine viruses in the cotton rat model was successfully confirmed (Table 17). PGP-42 T

TABLE 17
Assessment of replication of CPI-RSV-F and W3AΔSH-
RSV-F in the respiratory tract of cotton rats.

| | | | | Titer | Average titer |
|---|---|---|---|---|---|
| | Animal | | | (Log₁₀ | (Log₁₀ |
| Group | ID | Tissue | Day | PFU/mL) | PFU/mL) |
| Group K | 124880 | Nose | 4 | 3.7 | 3.9 |
| CPI-RSV-F | 124881 | Nose | 4 | 3.9 | |
| Day 4 | 124882 | Nose | 4 | 4.0 | |
| | 124880 | Lung | 4 | 3.8 | 3.5 |
| | 124881 | Lung | 4 | 3.3 | |
| | 124882 | Lung | 4 | 2.9 | |
| Group L | 124883 | Nose | 4 | 4.5 | 5.0 |
| W3AΔSH-RSV-F | 124884 | Nose | 4 | 4.9 | |
| Day 4 | 124885 | Nose | 4 | 5.2 | |
| | 124883 | Lung | 4 | 2.3 | 2.2 |
| | 124884 | Lung | 4 | 2.2 | |
| | 124885 | Lung | 4 | 1.7 | |

Animals were inoculated with 10⁶ PFU of CPI-RSV-F or W3A:ΔSH-RSV-F intranasally and sacrificed four days later. Lung and nose samples were collected and shipped to BLB for plaque assay.

### Evaluation of Vaccines for Efficacy and Safety in the Cotton Rat Model of RSV A/A2 Infection.

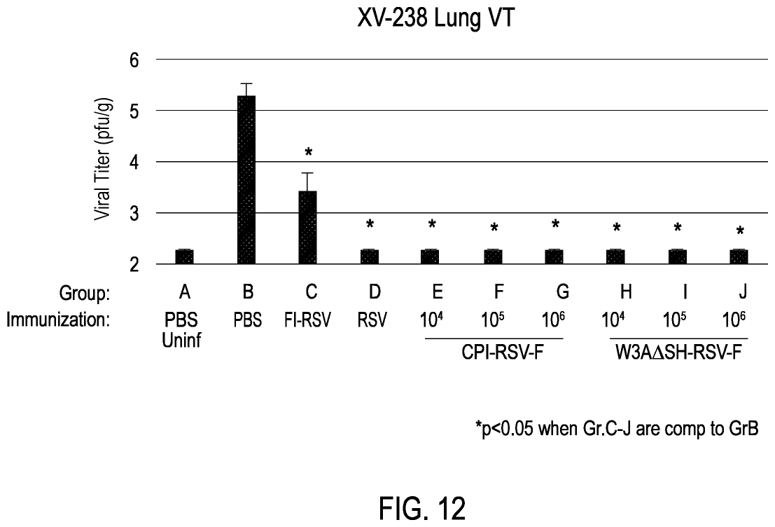

Lung viral titers: RSV A/A2 load in the lungs of cotton rats was evaluated 5 days after intranasal RSV challenge (FIG. 12). RSV-infected animals mock-immunized with PBS (Group B) showed a titer of 5.3 Log₁₀ PFU/g virus in the lungs and were used for comparison to all other RSV-infected groups (Groups C-J). No virus was detectable in the lungs of animals infected with RSV twice (Group D). FI-RSV immunization (Group C) moderately, but significantly reduced lung RSV titer to 3.4 Log₁₀ PFU/g. Immunization with CPI-RSV-F at doses 10⁴, 10⁵, or 10⁶ PFU (Groups E, F, and G, respectively) or with W3AΔSH-RSV-F at doses 10⁴, 10⁵, or 10⁶ PFU (Groups H, I, and J, respectively) reduced RSV load to an undetectable level in all vaccinated animals.

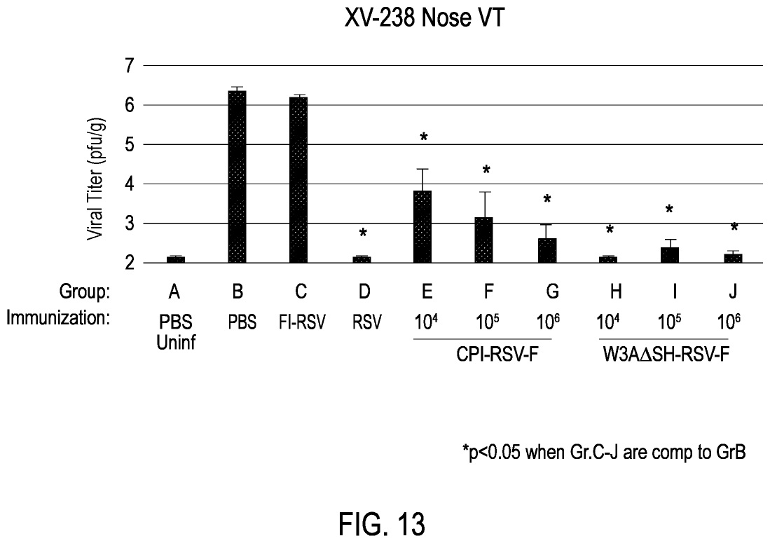

Nose viral titers: RSV A/A2 load in the nose of cotton rats was evaluated 5 days after intranasal RSV challenge (FIG. 13). RSV-infected animals mock-immunized with PBS (Group B) showed a titer of 6.19 Log 10 PFU/g virus in the nose and were used for comparison to all other RSV-infected groups (Groups C-J). No virus was detected in the nose of animals infected with RSV twice (Group D). FI-RSV immunization (Group C) had no effect on RSV load in the nose (6.02 Log 10 PFU/g). Immunization with CPI-RSV-F at doses of 10⁴, 10⁵, or 10⁶ PFU resulted in the dose-dependent reduction in viral load in the nose: 3.65, 3.00, and 2.44 PFU/g in Groups E, F, and G, respectively. Immunization with W3AΔSH-RSV-F at all three doses tested (Groups H-J) reduced RSV replication in the nose to an almost undetectable level, with only single-digit numbers of plaques in some animals in each group (1, 3, and 1 animal in Groups H, I, and J, respectively, had between 1 and 5 plaques).

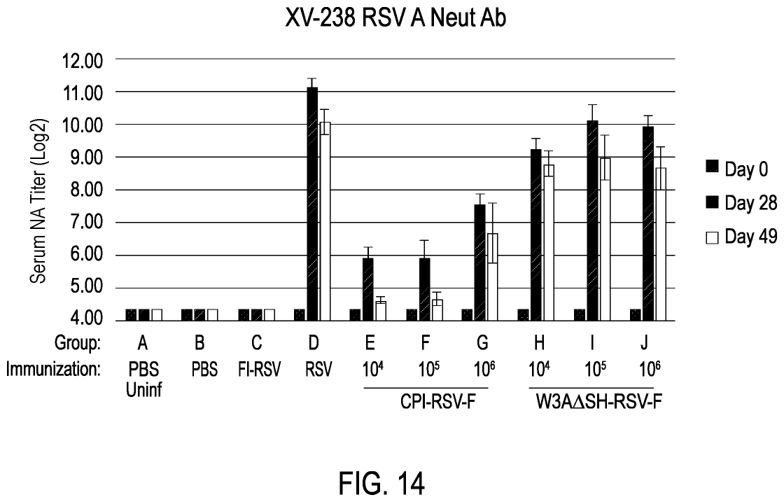

Serum RSV A/A2 Neutralizing Antibodies: Serum neutralizing antibodies against RSV A/A2 were measured in all animals prior to the start of experiment (day 0), 4 weeks after the first immunization (day 28), and three weeks later (day 49) (FIG. 14). Animals infected with RSV A/A2 (Group D) showed high titers of serum RSV A/A2 neutralizing antibodies four weeks after infection (day 28 data, 11.14 Log 2), which remained elevated seven weeks after infection (day 49 data, 10.07 Log 2). Immunization with CPI-RSV-F at doses of 10⁴, 10⁵, or 10⁶ PFU (Groups E, F, and G, respectively) resulted in NA titers of 5.92, 5.87, and 7.55 on day 28.

Animals immunized with the highest dose of vaccine tested (Group G, 10⁶ PFU) retained elevated NA titer by day 49 (6.65 Log 2), while NA levels in Groups E and F declined to 4.6 and 4.62 Log 2, respectively, by that time. Immunization with W3AΔSH-RSV-F at 10⁴, 10⁵, or 10⁶ PFU (Groups H-J) resulted in NA titer of 9.24, 10.11, and 9.91 Log 2 on day 28, respectively. The NA titer in these groups remained strongly elevated on day 49 (8.79, 8.97, 8.64 Log 2, respectively). No other group of animals showed detectable neutralizing antibodies against RSV A/A2.

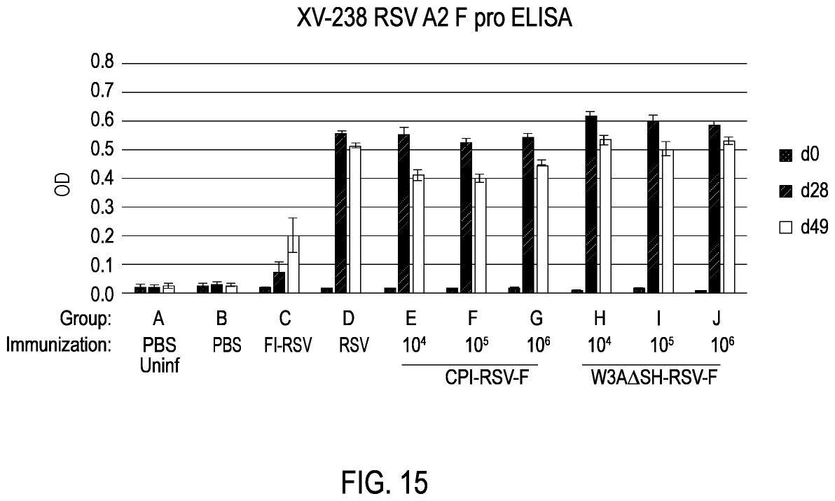

Serum RSV A/A2 Binding IgG Antibodies: Serum binding IgG antibodies against RSV A/A2 F protein were measured in all animals prior to the start of experiment (day 0), 4 weeks after the first immunization (day 28), and three weeks later (day 49) (FIG. 15). A small increase in binding IgG was visible in animals vaccinated with FI-RSV (Group C). Animals infected with RSV A/A2 (Group D) and all animals immunized with CPI-RSV-F (Groups E-G) or W3AΔSH-RSV-F (Groups H-J) had high levels of binding IgG on days 28 and 49 post-inoculation. No dose-dependency of IgG levels on either CPI-RSV-F (Groups E-G) or W3AΔSH-RSV-F (Groups H-J) doses was seen. IgG levels were slightly higher in W3AΔSH-RSV-F-immunized animals compared to animals immunized with CPI-RSV-F (Groups E-G) on both days 28 and 49.

Ling Histopathology: Pulmonary histopathology was evaluated in all animals 5 days after RSV A/A2 challenge (Data not shown). RSV-infected animals mock-immunized with PBS (Group B) or challenged with RSV twice (Group D) had moderate level of pathology. The highest level of pulmonary histopathology was detected in animals immunized with FI-RSV (Group C), with prominent increases in interstitial inflammation and alveolitis. Pathology of animals immunized with CPI-RSV-F in all doses (Groups E-G) did not exceed that seen in animals with secondary RSV infection (Group D). A group of animals immunized with W3AΔSH-RSV-F in the dose of 10⁵ PFU (Group I) had one animal (#124872) with elevated interstitial inflammation and alveolitis. The four other animals in that groups and all animals immunized with the other two doses of vaccines (Groups H and J) did not exhibit interstitial inflammation and alveolitis.

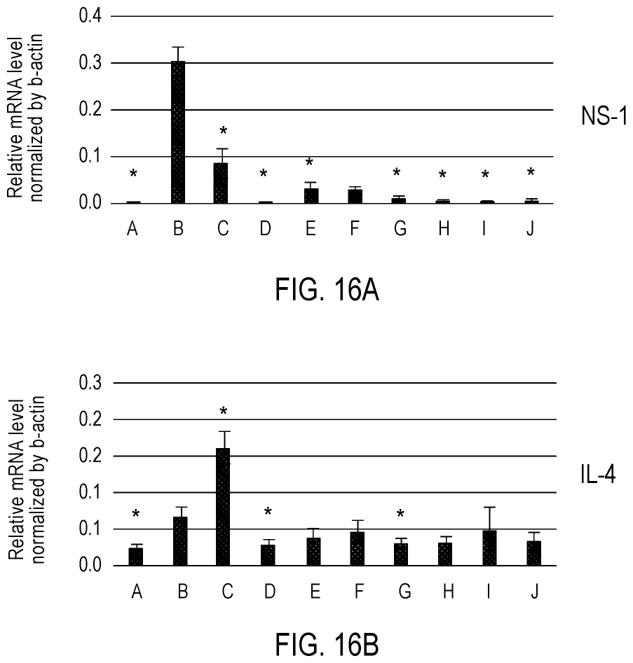

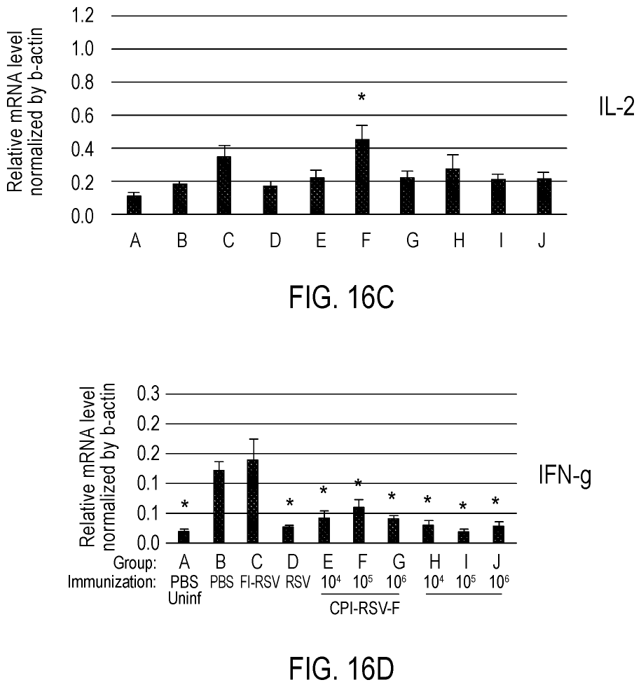

qPCR Results: Expression of RSV NS1, IL-4, IL-2, and IFN-γ mRNA was evaluated in lung samples collected on day 5 after RSV A/A2 challenge and normalized by the level of β-actin mRNA in each sample (FIG. 16A-16D). Expression of NS-1 mRNA was strongly reduced by both vaccines, with W3AΔSH-RSV-F being slightly more efficacious at reducing lung NS1 mRNA compared to CPI-RSV-F administered at the two lower doses (10⁴ and 10⁵ PFU). FI-RSV immunization (Groups C) resulted in moderate reduction in lung RSV NS1 mRNA level, but significantly increased IL-4 mRNA level. IL-4, IL-2, and IFN-γ mRNA levels in animals immunized with CPI-RSV-F (Groups E-G) or W3AΔSH-RSV-F (Groups H-J) overall did not exceed that seen in animals with primary RSV infections (Group B). Several animals in the group immunized with the intermediate dose of CPI-RSV-F (Group F, 10⁵ PFU dose) showed slightly elevated IL-2 and IFN-γ mRNA levels compared to animals with secondary RSV infection (Group D). One animal in the group immunized with the intermediate dose of W3AΔSH-RSV-F (Group I, 10⁵ PFU dose, animal ##124872) had elevated IL-4 mRNA. Coincidentally, this same animal showed elevated alveolitis and interstitial inflammation in histopathology evaluation.

### Example 5: Phase I Clinical Trial of PIV5-RSV Vaccine—RSV Antibody and CMI Response Rates

### Materials and Methods

Participants and study conduct: A total of 30 subjects were enrolled in this phase 1 trial and all received a single intranasal dose of the PIV5-RSV vaccine at 10^{7.5} PFU. The vaccine was administrated as a 0.25 mL spray to each nostril (total volume 0.5 mL) using a MAD Nasal™ Intranasal Mucosal Atomization Device (Teleflex MAD300). The mean ages in Group 1 (planned participant age 18-59 years and actual enrollee age 33-59 years) and Group 2 (planned participant age 60-75 years and actual enrollee age 61-75 years) were 45 years and 67 years, respectively (Table 18). The majority of participants were female (70%). Most participants in the two groups were White (73% and 80% in Groups 1 and 2, respectively). All except one participant completed the study; and this participant in Group 1 was lost to follow-up after Day 7.

TABLE 18
Trial cohort demographics

| Group | Group 1 | Group 2 |
|---|---|---|
| N | 15* | 15 |
| Mean age (range) | 44.9 (33, 59) | 66.8 (61, 75) |
| Male/female | 4/11 | 5/10 |
| Race/Ethnicity | 11 White (73%), others- | 12 White (80%), others- |
| | Black/African American | Black/African American |
| | and American Indian | and Asian |

*One participant dropped out of the study on Day 8 (lost to follow-up).

Study design and vaccination: The phase 1 clinical trial of PIV5-RSV vaccine (also referred to as BLB201; Clinical Trials NCT05281263) was approved by the Advarra central IRB and conducted at two study sites in the US. Participants were recruited to two study cohorts, healthy young adults (Group 1, 33-59 years old) and healthy older adults (Group 2, 61-75 years old). Participants of childbearing potential were required to practice contraceptive measurement to prevent pregnancy. Exclusion criteria included any live vaccine within the 30 days prior to trial vaccine, any prior receipt of any investigational RSV vaccine or any PIV5-based vaccine (CVXGA1) that was actively enrolling during the trial period and known infection with human immunodeficiency virus, hepatitis B virus, or hepatitis C virus. A full list of inclusion and exclusion criteria can be found on the clinical trials website. Participants were not prescreened for their RSV serum antibody levels. Eligible participants were administered on Day 1, a single dose at a concentration of 10^{7.5} plaque forming unit (PFU) of PIV5-RSV vaccine, as a 0.25 mL spray to each nostril (total volume 0.5 mL) using a MAD Nasal™ Intranasal Mucosal Atomization Device (Teleflex MAD300) and observed for 30 minutes immediately after dosing. In addition, subjects were asked to maintain a memory aid for solicited systemic AEs and local reactions during the week after vaccination. Four sentinel participants were dosed first in each group, and their safety data was reviewed by the safety monitory committee (SMC) before the remainder of the participants in the group were enrolled.

Primary outcome measures included (i) solicited AEs (Day 1-8), and (ii) unsolicited AEs (Days 1-29). Secondary outcome measures included (i) serum IgG titers to RSV protein (Days 15 and 29) (ii) SAEs (Days 1-181) and (iii) AEs of special interest (AESIs) including x-onset chronic medical conditions, and medically-attended AEs (Days 1-181).

Immunogenicity assessment: Blood and nasal samples were collected at baseline (Day 1, pre vaccination), Day 15, and Day 29 post-vaccination. To reduce sample variation, samples from the same participant were run on the same plate in a blinded fashion in all the assays. Serum RSV nAb levels were determined by a qualified RSV A2 microneutralization (MN) assay based on RSV-A2-rLuc reporter virus (45). Briefly, serially 2-fold diluted serum samples in quadruplicate from a starting dilution of 1:100 were incubated with 175±75 PFU RSV-rLuc for 1 hr, and infected Vero cells on 96-well, white-walled, clear bottom plates. After 20 to 24 hr incubation, the cells were lysed using Renilla-Glo Luciferase assay (Promega) and luciferase signals were read on a SpectraMax iD3 multi-mode microplate reader (Molecular Devices). RSV nAb titer was defined as reciprocal dilution that inhibited at least 50% signal of the virus control as determined by 5PL curve fitting using Prism (Version 9.5.1 for macOS, GraphPad Software). The RSV nAb titer was converted to international units based on the standard serum (16/284) obtained from NIBSC (London, UK).

RSV F specific serum IgG and IgA antibody, and nasal IgA antibody levels were determined by ELISA assay using 25 ng/well of purified RSV F protein (SinoBiological, cat #11049-V08B) and 2-fold serial dilutions in blocking buffer (5% milk/0.5% BSA in 1×KPL wash buffer (Seracare) in duplicate. End point titer was calculated by 4PL curve fitting using Prism and reported as reciprocal dilution. PIV5-specific IgG and nAb titers were determined by ELISA assay using PIV5 virus coated plates and by PIV5-rLuc-based MN assay in Vero cells, respectively. The PIV5 IgG end point titer was calculated by 4PL curve fitting and reported as reciprocal dilution. The PIV5 MN assay was performed similarly to the RSV-rLuc based MN assay, except in duplicate instead of quadruplicate. Analysis of PIV5 MN was identical to the RSV-rLuc based MN assay and the nAb titer was defined as the reciprocal dilution that inhibited at least 50% signal of the virus control.

Antigen-specific T-cell frequencies were evaluated by intracellular cytokine staining assay using cryopreserved peripheral blood mononuclear cells (PBMCs) isolated from whole blood on Day 1 (pre-vaccine dosing), Day 15, and Day 29. One million cryopreserved PBMCs were thawed in complete 10% FBS RPMI medium and rested overnight at 37° C. before being incubated with an RSV F peptide pool from GenScript at a final concentration of 1 μg/mL in the presence of 1 μg/mL anti-CD28 ECD (Beckman Coulter, clone CD28.2), anti-CD107a FITC (BD Biosciences, clone H4A3) and anti-CD49d (BD Biosciences, clone 9F10). In addition, PBMCs were stimulated with 1 μL of complete medium with 0.5% dimethyl sulfoxide (DMSO, negative control corresponding to the DMSO concentration of the RSV-F peptide pool) or 1 μL of PMA/ionomycin (25 ng/mL PMA and 1 μg/mL ionomycin) for negative and positive controls, respectively. After 2 hrs incubation at 37° C., 10 μg/mL brefeldin A (BD Biosciences) were added and the cells were incubated for another 4 hrs. The cells were washed with PBS and incubated with Aqua-Viability dye (Invitrogen) at room temperature for 15 mins. The cells were washed with PBS supplemented with 2% fetal bovine serum (FBS), and surface stained at 4° C. for 30 mins with anti-CD3 Alexa700 (BD Biosciences, clone SP34-2), anti-CD4 BV605 (BD Biosciences, clone L200), anti-CD8 BV450 (BD Biosciences, clone RPA-T8), and anti-CD95 PE-Cy5 (BD Biosciences, clone DX2). The cells were washed with PBS with 2% FBS, fixed with Cytofix/Cytoperm (BD Biosciences), permeabilized with 1×Perm/Wash (BD Biosciences), and incubated with anti-IFN-γ PE-Cy7 (BD Biosciences, clone B27), anti-TNF-α APC-Cy7 (BioLegend, clone Mab11), anti-IL-13 PE (Miltenyi Biotec, clone JES10-5A2.2) and anti-MIP-1β APC (eBioscience, clone FL34Z3L) antibodies at 4° C. for 30 mins. The cells were washed with 1×Perm/Wash and PBS with 2% FBS then resuspended in PBS/2% formaldehyde for acquisition on a BD FACSAria Fusion cell sorter. CD3+ cells were gated for CD4⁺ and CD8⁺ T-cells and separated into memory and naïve cells with CD28 and CD95. The net percentage of cytokine-secreting cells was determined by subtraction of the values obtained with DMSO-stimulated samples (negative control). T cell frequencies were considered positive if the detected frequency of cytokine positive CD4⁺ or CD8⁺ T cells was >0.1% after subtraction of baseline. Data were analyzed using FlowJo software (version 10). A Boolean combination and SPICE software were used to determine polyfunctional responses of CD4⁺ and CD8⁺ T cells producing two or more cytokines.

Statistical analysis: Statistical analysis was performed using GraphPad Prism software (Version 9). Given the small sample size, statistical analysis was mostly descriptive and summative. P values were used to show difference of potential significance at a 0.05 significance level. Two-group comparisons of RSV-specific CMI responses and antibody responses were evaluated by the Wilcoxon matched-pairs signed rank test (Day 15 or Day 29 vs Day 1, or Day 15 vs Day 29). Antibody titer rises of 1.5-fold post-vaccination vs baseline was considered meaningful because assay characteristics showed a change of 1.2-1.3-fold (95% confidence) to be a notable change when samples from a single participant were tested on the same plate.

### Results

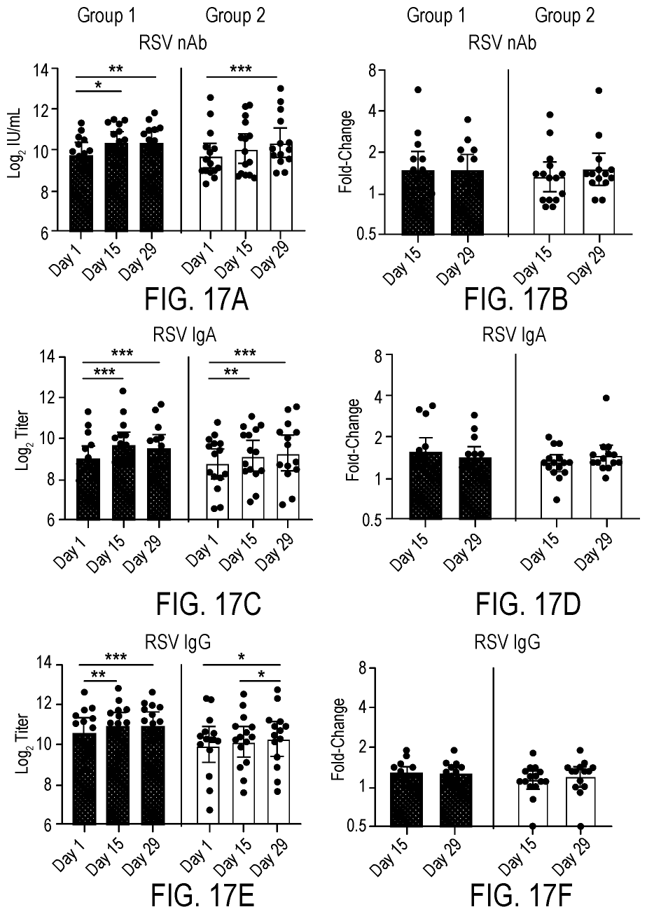

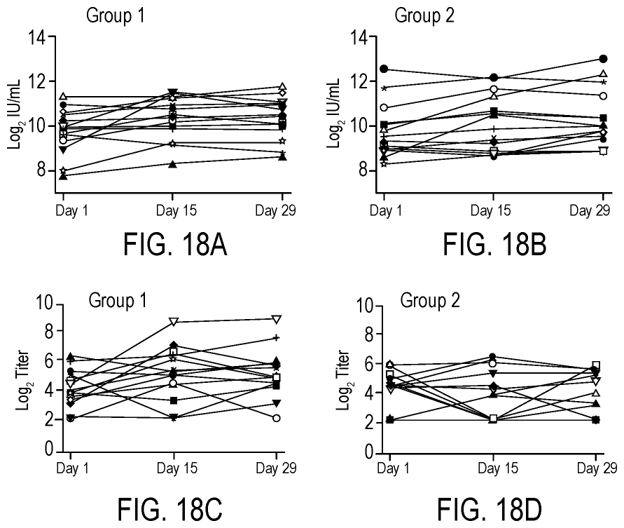

Serum antibody titers: All participants were seropositive for RSV neutralizing Abs (nAbs) at baseline and the titer changes are presented in FIGS. 17A-17F and FIGS. 18A-18B. In Group 1, the nAbs geometric mean titer (GMT) was 880 (9.8 log₂) at baseline and increased to 1316 (10.4 log₂) at 2-weeks and 1312 (10.4 log₂) at 4-weeks after vaccination (P<0.05), reflecting a geometric mean fold rise (GMFR) of 1.5. RSV-nAb seroresponses (≥1.5-fold rise) were identified in 7/14 (50%) participants. In Group 2, the nAb GMT was 850 (9.7 log₂) at baseline, similar to that in Group 1, increased to 1103 (10.1 log₂) at 2-weeks (P<0.05), and 1372 (10.4 log₂) at 4-weeks after vaccination (P<0.05), reflecting geometric mean fold rise (GMFRs) of 1.3 and 1.5, respectively. RSV-nAb seroresponses were identified in 6/15 (40%) participants in this group (Table 19) (individual nAb titer changes were shown in FIGS. 18A-18D). Notably, RSV-nAb seroresponses were identified in all 5 participants positive for vaccine-virus shedding, suggesting a potential correlation between replication and systemic antibody responses.

TABLE 19
Summary of RSV antibody and CMI response rates

| | | Group 1 | Group 2 |
|---|---|---|---|
| | | responders | responders |
| | Immune | n/N | n/N |
| Sample | parameter | (% responder) | (% responder) |
| Serum | RSV nAb | 7/14 (50%) | 6/15 (40%) |
| | RSV F IgA | 6/14 (43%) | 5/15 (36%) |
| | RSV F IgG | 3/14 (21%) | 1/15 (7%) |
| | PIV5 nAb | 7/14 (50%) | 10/15 (67%) |
| | PIV5 IgG | 12/14 (86%) | 13/15 (87%) |
| Nasal Swab | Nasal RSV F IgA | 9/14 (64%) | 5/15 (33%) |
| PBMC | Total CMI | 13/14 (93%) | 15/15 (100%) |
| | CD8 CTL | 12/14 (86%) | 13/15 (87%) |

A positive seroresponse was defined as ≥1.5-fold rise from baseline for RSV F IgG and IgG. A positive nasal IgA antibody response was defined as ≥2-fold rise over baseline. CMI response was defined as >0.1% rise of sum of individual T cell secreting >1 cytokine after subtraction of baseline (pre-vaccination). CTL response was defined by the change in INF-γ producing CD8⁺ cells. Abbreviations: CMI, cell-mediated immunity; CTL, cytotoxic T lymphocyte: PBMC, peripheral blood mononuclear cell; PIV5, parainfluenza virus type 5: RSV, respiratory syncytial virus.

All participants were also seropositive for RSV-F-specific serum IgA and IgG Abs, at baseline (FIGS. 17A-17F). In Group 1. F-specific serum IgA GMT increased from 530 (9.0 log₂) at baseline to 821 (9.7 log₂) and 756 (9.6 log₂) at 2- and 4-weeks after vaccination, respectively. F-specific serum IgG GMT increased from 1640 (10.7 log₂) at baseline, to 2049 (11.0 log₂) at 2-weeks and 2055 (11.0 log₂) at 4-weeks after vaccination. F-specific serum IgA and IgG seroresponses (≥1.5-fold) were identified in 6/14 (43%) and 3/14 (21%) participants, respectively (Table 19). In Group 2. F-specific serum IgA GMT increased from 445 (8.8 log₂) at baseline, to 582 (9.2 log₂) and 641 (9.3 log₂) at 2- and 4-weeks after vaccination, respectively. F-specific serum IgG GMT increased from 1088 (10.1 log₂) at baseline, to 1198 (10.2 log₂) and 1325 (10.4 log₂) at 2- and 4-weeks after vaccination. F-specific IgA and IgG seroresponses were identified in 5/15 (33%) and 1/15 (7%) participants, respectively (Table 19).

Overall, the results suggested the PIV5-RSV vaccine boosted RSV-specific serum Ab levels in young adults (33-59-year old) and elderly (61-75-year old), though with greater magnitude in adults vs elderly. Interestingly, the fold increases in RSV-specific Ab titers inversely correlated with baseline titers. It is possible that the higher baseline titers were close to maximum levels.

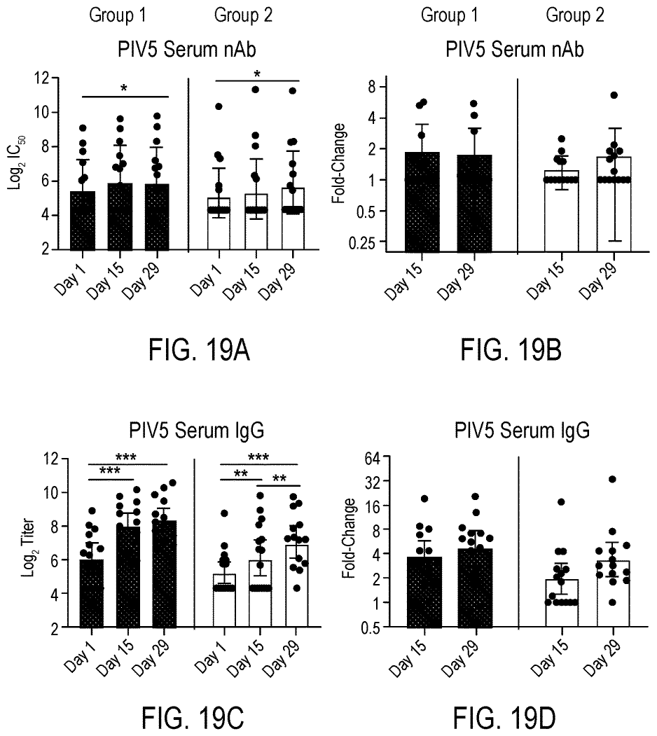

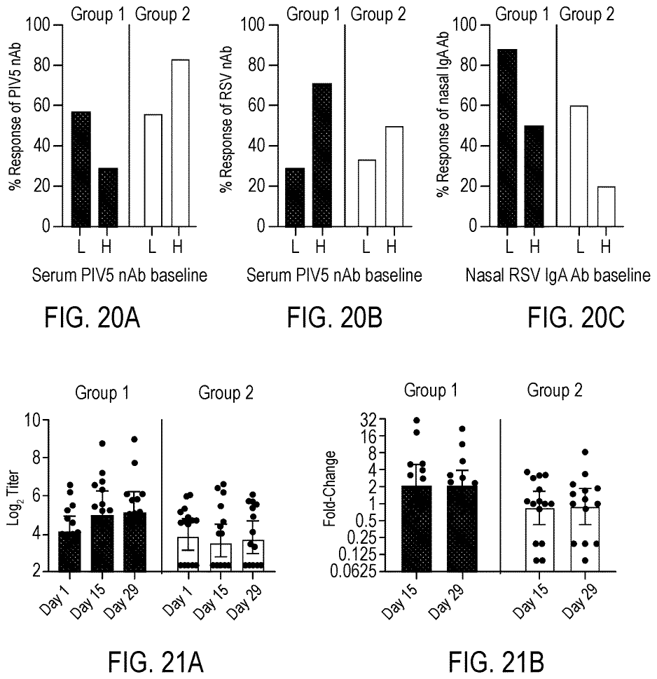

We next examined vector-specific immune responses. For PIV5 nAbs at baseline, 15/29 (52%) participants were seropositive as defined by a titer of 1:10 (FIGS. 19A-19B). This level of background seropositivity likely reflects passive exposure to PIV5 through exposure to dogs receiving the kennel cough vaccine. In Group 1, the PIV5-nAb GMT increased from 26 (4.7 log₂) at baseline, to 48 (5.6 log₂) and 51 (5.7 log₂) at 2- and 4-weeks after vaccination, respectively, reflecting a geometric mean fold rise of 1.8 and 2.0 respectively. PIV5-nAb seroresponses (≥1.5-fold rise) were identified in 7/14 (50%) participants (Table 19). In Group 2, the PIV5-nAb GMT increased from 19 (4.2 log₂) at baseline, to 34 (5.1 log₂) and 44 (5.5 log₂) at 2- and 4-weeks after vaccination, respectively, reflecting a GMFRs of 1.8 and 2.2 respectively. PIV5-nAb seroresponses (≥1.5-fold rise) were identified in 10/15 (67%) participants. Similar kinetics were observed with PIV5-specific IgG titers (FIGS. 19C-19D), but with greater GMFRs (≤4.6 fold in Group 1 and ≤3.4 fold in Group 2) and seroresponses (86% in Group 1 and 87% in Group 2). By comparing participants with low PIV5 nAb titers with those with high PIV5 nAb titers, baseline PIV5 nAb titers had no clear effect on PIV5-nAb seroresponse rate and appeared to not inhibit RSV-nAb seroresponse rate (FIGS. 20A-20C).

RSV F-specific IgA antibody titers in nasal swabs: At baseline, RSV F-specific IgA antibody (Ab) titers in nasal swabs were variable ranging from below the LOD to 514 (9.0 log₂) (FIG. 21A). In Group 1, the F-specific nasal IgA geometric mean titer (GMT) was 19 (4.2 log₂) at baseline and increased to 40 (5.3 log₂) at 2- and 4-weeks after vaccination (P<0.05), reflecting a GMFR of 2.1 (FIG. 21B). F-specific nasal IgA responses (22-fold rise) were identified in 9/14 (64%) participants (Table 19). In Group 2, the baseline F-specific nasal IgA GMT of 18 (4.2 log₂) was similar to that in Group 1, but GMTs were not boosted (P>0.05) 2-weeks and 4-weeks after vaccination (FIGS. 21A and 21B). However, F-specific nasal IgA responses were identified in 5/15 (33%) participants in Group 2 (individual's nasal IgA antibody changes were shown in FIGS. 18C-18D). In Groups 1 and 2, F-specific nasal IgA responses were identified in 4/5 participants positive for vaccine-virus shedding.

Overall, the results suggested that the PIV5-RSV vaccine boosted RSV-specific nasal IgA levels in adults and elderly, but to a lesser degree in elderly. Similar to results seen with systemic antibody responses, the fold increases in F-specific nasal IgA titers in both groups inversely correlated with baseline titers (FIGS. 20A-20C).

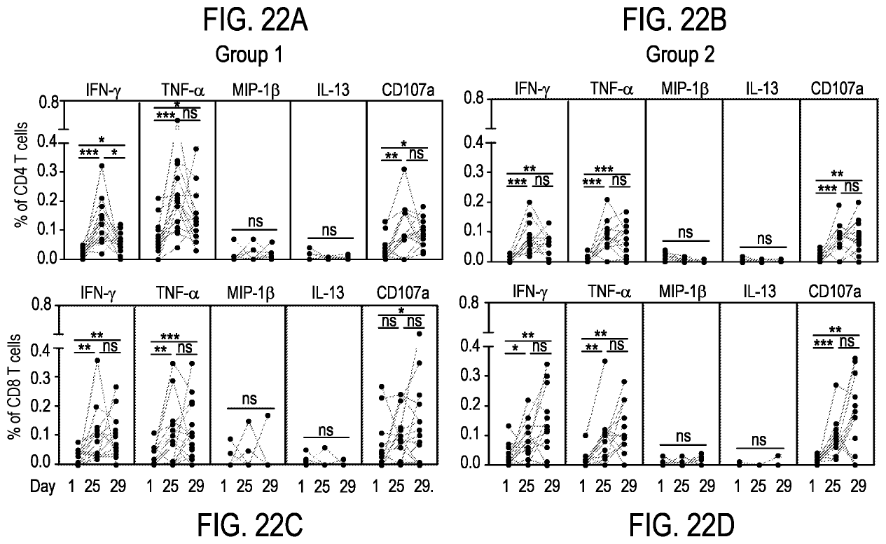

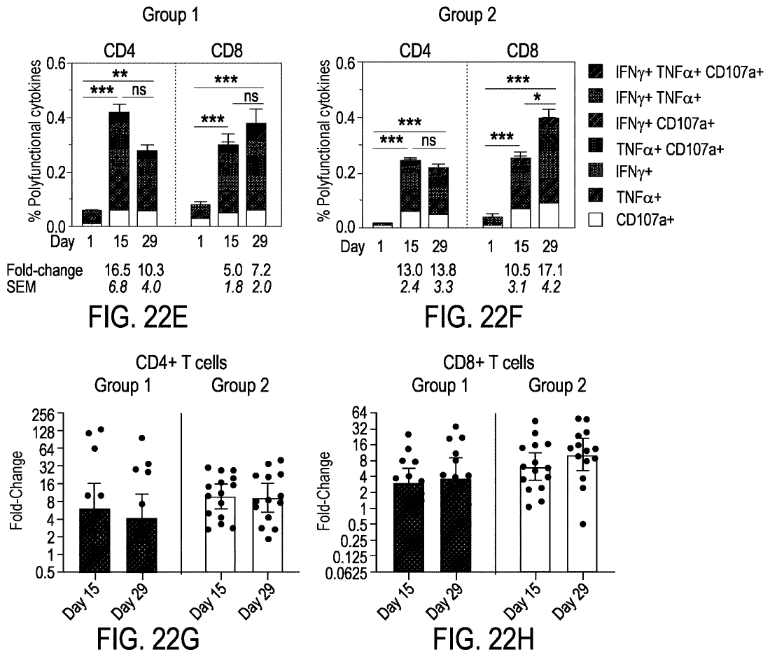

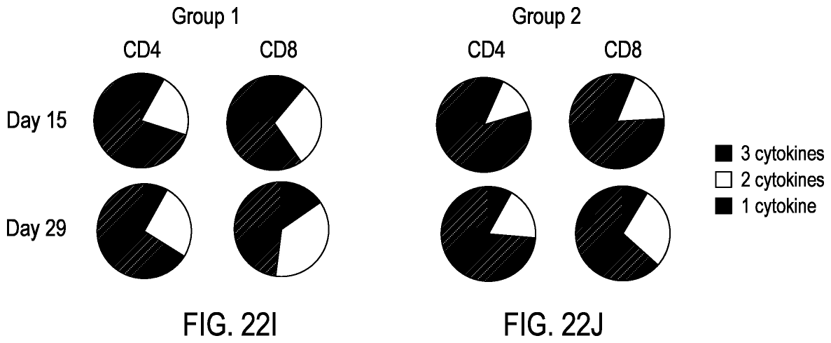

Cell mediated immunity at baseline. RSV F-specific CD4⁺ and CD8⁺ T cells were detected, based on the expression of Th1 or cytotoxic T-cell cytokines/markers IFN-γ, TNF-α, MIP-1β and CD107a, and on the expression of Th2 cytokine IL-13 (FIG. 22A-22D). In Group 1, the mean percentages of F-specific CD4⁺ T cells expressing at least one of the Th1/cytotoxic markers increased from 0.06% at baseline to a maximum of 0.42% 2-weeks after vaccination (P<0.001; FIG. 22E-22F), and the mean percentages of the same phenotypes of CD8⁺ T cells increased from 0.08% at baseline to a maximum of 0.38% 4-weeks after vaccination (P<0.001). In Group 2, the mean percentages of the same phenotypes of CD4⁺ T cells increased from 0.02 to a maximum of 0.26%, 2-weeks after vaccination (P<0.001), and CD8⁺ T cells increased from 0.04 to a maximum of 0.40% 4-weeks after vaccination (P<0.001). In Groups 1 and 2, F-specific cell-mediated immunity (CMI) responses to the PIV5-RSV vaccine (defined as an increase from baseline of >0.1% of T cells expressing at least 2 Th1/cytotoxic biomarkers) were identified in 13/14 (93%) and 15/15 (100%) participants, respectively (Table 19). And F-specific cytotoxic T-lymphocyte (CTL) responses to the PIV5-RSV vaccine (defined as an increase from baseline of >0.1% of CD8⁺ T cells expressing IFN-γ) were identified in 12/14 (86%) and 13/15 (87%) participants, respectively. This suggested that the PIV5-RSV vaccine enhanced the production of F-specific Th1 (CD4⁺) and cytotoxic (CD8⁺) T cells in nearly all participants. Even though numbers of CD4⁺ and CD8⁺ T cells were similar after immunization in both groups, geometric mean of CD4⁺ T cells rose to 6.1- and 9.1-fold by day 15 for Group 1 and 2 respectively, and geometric mean of CD8⁺ T cells rose to 3.6- and 10-fold by day 29 for Group 1 and 2 respectively (FIG. 22G-22H), reflecting lower baseline for Group 2 than for Group 1. This result suggests that in contrast to nasal IgA response, similar levels of CMI in adults and elderly was induced by the PIV5-RSV vaccine.

In both Group 1 and Group 2, T cells expressing a single Th1/cytotoxic biomarker were more frequent than those expressing at least two Th1/cytotoxic markers (FIG. 22I-22J). However, T-cells co-expressing at least two Th1/cytotoxic biomarkers tended to be more frequent in Group 1 vs Group 2, suggesting that the PIV5-RSV vaccine may induce better quality of antigen specific effector/memory T cells in the younger age group, based on polyfunctional Th1/cytotoxic responses.

In contrast to Th1 and cytotoxic phenotypes, the percentages of F-specific CD4⁺ T cells and CD8⁺ T cells expressing IL-13 did not increase after vaccination in either Group 1 or Group 2 (FIG. 22A-22D), indicating that there was no Th2-biased response to the PIV5-RSV vaccine, which has been identified as a potential risk factor for vaccine-associated enhanced RSV disease (F. P. Polack, et al., J Exp Med 196, 859-865 (2002); B. Bagga, et al., J Infect Dis 212, 1719-1725 (2015)).

The complete disclosure of all patents, patent applications, and publications, and electronically available material (including, for instance, nucleotide sequence submissions in. e.g., GenBank and RefSeq, and amino acid sequence submissions in. e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### REFERENCES

1. Phan S I, Zengel J R, Wei H, et al. Parainfluenza virus 5 expressing wild-type of prefusion respiratory syncytial virus (RSV) fusion protein protects mice and cotton rats from RSV challenge. J of Virology 2017:91(19); e00560-17.
2. Wang D, Phan S, DiStephano D J, et al. A single-dose recombinant parainfluenza virus 5-vectored vaccine expressing respiratory syncytial virus (RSV) F or G protein protected Cotton rats and African green monkeys from RSV challenge. J of Virology 2017:91(11); e00066-17.
3. Phan S I, Chen Z, Xu P. et al. A respiratory syncytial virus (RSV) vaccine based on parainfluenza virus 5 (PIV5). Vaccine 2014:32; 3050-3057.
4. Young D. Wignall-Fleming E B, Busse D C, et al. The switch between acute and persistent paramyxovirus infection caused by single amino acid substitutions in the RNA polymerase P subunit. PLoS Pathogens 2019 Feb. 11:15(2):e1007561. doi: 10.1371/journal.ppat.100756.
5. Seth et al. Mutations in the cytoplasmic domain of a paramyxoviruses fusion glycoprotein rescue syncytium formation and eliminate the HN requirement for membrane fusion. J. of Virology 2003, 167-178.
6. Tong et al. Regulation of Fusion Activity by the Cytoplasmic Domain of a Paramyxovirus F Protein. J. of Virology 2002, 301:322-333.
7. Schmitt, A. P., Leser, G. P., Waning, D. L. & Lamb, R. A. Requirements for Budding of Paramyxovirus Simian Virus 5 Virus-Like Particles. Journal of Virology 76, 3952-3964, doi:10.1128/jvi.76.8.3952-3964.2002 (2002).
8. Waning, D. L., Schmitt, A. P., Leser, G. P. & Lamb, R. A. Roles for the cytoplasmic tails of the fusion and hemagglutinin-neuraminidase proteins in budding of the paramyxovirus simian virus 5. JVirol 76, 9284-9297 (2002).
9. F. P. Polack, M. N. Teng, P. L. Collins, G. A. Prince, M. Exner, H. Regele, D. D. Lirman, R. Rabold, S. J. Hoffman. C. L. Karp, S. R. Kleeberger. M. Wills-Karp, R. A. Karron, A role for immune complexes in enhanced respiratory syncytial virus disease. J Exp Med 1%, 859-865 (2002).
10. B. Bagga, J. E. Cehelsky, A. Vaishnaw, T. Wilkinson, R. Meyers, L. M. Harrison, P. L. Roddam, E. E. Walsh, J. P. DeVincenzo, Effect of Preexisting Serum and Mucosal Antibody on Experimental Respiratory Syncytial Virus (RSV) Challenge and Infection of Adults. J Infect Dis 212, 1719-1725 (2015).

What is claimed is:

1. A viral expression vector comprising a parainfluenza virus 5 (PIV5) genome having a heterologous nucleic acid sequence with at least 95% sequence identity to SEQ ID NO: 1, wherein the viral expression vector expresses a heterologous polypeptide comprising a live recombinant canine parainfluenza (CPI) vector backbone engineered to express a RSV F protein as target antigen.

2. The viral expression vector of claim 1, wherein the RSV F protein is encoded by a wildtype or mutated RSV F protein gene.

3. The viral expression vector of claim 1, wherein the RSV F protein gene is codon optimized for expression in a human subject.

4. The viral expression vector of claim 1, wherein the RSV F protein gene is inserted between a SH and HN junction of a CPI antigenomic cDNA.

5. The viral expression vector of claim 4, wherein the CPI antigenomic cDNA is sequenced with primers having a nucleic acid sequence with at least 95% sequence identity to SEQ ID NO: 2, 3, 4, 5, 6, 7, 8, 9, 10, 11, 12, 13, 14, 15, 16, 17, 19, or 19.

6. The viral expression vector of claim 1, wherein the parainfluenza (CPI) vector backbone engineered to express the RSV F protein comprises a 22 amino acid extension as part of its cytoplasmic tail.

7. A pharmaceutical composition comprising a parainfluenza virus 5 (PIV5) viral expression vector having a heterologous nucleic acid sequence with at least 95% sequence identity to SEQ ID NO: 1, wherein the viral expression vector expresses a heterologous polypeptide comprising a live recombinant canine parainfluenza (CPI) vector backbone engineered to express a RSV F protein as a target antigen.

8. The pharmaceutical composition of claim 7, wherein the RSV F protein is encoded by a wildtype or mutated RSV F protein gene.

9. The pharmaceutical composition of claim 7, wherein the RSV F protein gene is codon optimized for expression in a human subject.

10. The pharmaceutical composition of claim 7, wherein the RSV F protein gene is inserted between a SH and HN junction of a CPI antigenomic cDNA.

11. The pharmaceutical composition of claim 10, wherein the CPI antigenomic cDNA is sequenced with primers having a nucleic acid sequence with at least 95% sequence identity to SEQ ID NO: 2, 3, 4, 5, 6, 7, 8, 9, 10, 11, 12, 13, 14, 15, 16, 17, 19, or 19.

12. The pharmaceutical composition of claim 7, wherein the parainfluena (CPI) vector backbone engineered to express the RSV F protein comprises a 22 amino acid extension as part of its cytoplasmic tail.

13. The pharmaceutical composition of claim 7, wherein the live recombinant canine parainfluenza (CPI) vector backbone engineered to express the RSV F protein is a prophylactic vaccine against RSV infection.

14. A method of inducing an immune response in a subject having RSV comprising administering a prophylactic vaccine against RSV infection, wherein the vaccine comprises a the pharmaceutical composition of claim 7.

15. The method of claim 14, wherein the vaccine induces RSV F-protein specific serum antibodies and cell mediated responses.

16. The method of claim 15, wherein the RSV specific cell mediate response comprises an increased expression of CD4⁺ T and CD8⁺ T cells.

17. The method of claim 15, wherein the RSV-F specific serum antibodies and cell mediated responses are associated with a reduced incidence of an RSV-induced pathological lung response compared to an immune response obtained by administering a formalin-inactivated RSV (FI-RSV).

18. The method of claim 17, wherein the pathological lung response is selected from a group consisting of: peribronchiolitis, perivasculitis, interstitial pneumonia, and alveolitis.

19. The method of claim 14, wherein the vaccine is administered intranasally, intramuscularly, topically, or orally.

20. The method of claim 14, wherein the vaccine is administered in a single or multiple dose regimen.


SEQUENCE LISTING (19 sequences)

SEQ ID NO: 1 - DNA, 17076 bp, synthetic construct
  misc_feature 1..17076: note = Synthetic: CPI-RSV-F 5' to 3'nucleic acid sequence
  source 1..17076: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattttgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatggagctgctgatcctgaaagccaacgctattaccacaatcctgaccgccgtgacattctgctttgcttccggacagaatatcaccgaggagttctaccagtcaacatgtagcgccgtgagcaaggggtacctgagtgcactgcgaaccggatggtatacatccgtgattactatcgagctgtctaacatcaagaaaaacaaatgcaatggcactgacgccaagatcaagctgatcaagcaggagctggataagtacaaaaatgctgtgaccgaactgcagctgctgatgcagagcactccagctaccaacaatcgcgcacggagagaactgccccggttcatgaactataccctgaacaatgcaaagaaaacaaatgtgactctgtccaagaaacgcaagaggcgcttcctgggatttctgctgggcgtggggtctgcaatcgccagtggagtggccgtctctaaagtcctgcacctggagggcgaagtgaacaagatcaaatcagcactgctgagcacaaacaaggccgtggtcagtctgtcaaatggcgtgtccgtcctgacttctaaggtgctggacctgaaaaattatattgataagcagctgctgcctatcgtcaacaaacagagctgttccatttctaatatcgagaccgtgatcgagttccagcagaagaacaatagacttctagagattacaagggaattttctgtgaacgctggcgtcactacccccgtgagtacctacatgctgacaaatagtgagctgctgtcactgattaacgacatgcctatcaccaatgatcagaagaaactgatgagtaacaatgtgcagatcgtcagacagcagagttactcaatcatgtcaatcattaaggaggaagtcctggcctacgtggtccagctgccactgtatggcgtgatcgacactccctgctggaaactgcatacctcacctctgtgcacaactaacaccaaggaggggagcaatatctgcctgacacgaactgaccggggatggtactgtgataacgccggcagcgtgtccttctttccccaggctgagacctgcaaggtccagtccaacagggtgttctgtgacactatgaatagcctgaccctgccttccgaagtcaacctgtgcaatgtggacatctttaatccaaagtacgattgtaagatcatgaccagcaagacagatgtcagctcgagtgtgattacatcactgggggctatcgtgagctgctacggaaagactaaatgtaccgcaagcaacaaaaatcgcggcatcattaagaccttcagtaacgggtgcgactatgtctcaaacaagggcgtggatacagtgagcgtcgggaacactctgtactatgtcaataagcaggagggaaaatccctgtacgtgaagggcgaaccaatcattaacttctatgaccccctggtgttcccttccgacgagtttgatgcctctattagtcaggtgaacgaaaaaatcaatcagagcctggcttttattcggaagtccgatgagctgctgcacaacgtgaatgcagtcaagtctaccacaaacattatgatcactaccatcattatcgtgattatcgtcattctgctgtccctgatcgcagtggggctgctgctgtactgtaaagccagatctacacccgtgactctgtctaaggatcagctgagtggaattaacaatatcgcctttagcaactgataggctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 2 - DNA, 25 bp, synthetic construct
  misc_feature 1..25: note = Synthetic primer: CVL1
  source 1..25: mol_type = other DNA
accaaggggaaaatgaagtggtgac

SEQ ID NO: 3 - DNA, 24 bp, synthetic construct
  misc_feature 1..24: note = Synthetic primer: CVL6
  source 1..24: mol_type = other DNA
ggagaagctcagatcagtgggatc

SEQ ID NO: 4 - DNA, 25 bp, synthetic construct
  misc_feature 1..25: note = Synthetic primer: CVL5
  source 1..25: mol_type = other DNA
acagtggattccataaggttcctgc

SEQ ID NO: 5 - DNA, 27 bp, synthetic construct
  misc_feature 1..27: note = Synthetic primer: CVL11
  source 1..27: mol_type = other DNA
cactgacagaaatgtcggaatgaagag

SEQ ID NO: 6 - DNA, 25 bp, synthetic construct
  misc_feature 1..25: note = Synthetic primer: CVL10
  source 1..25: mol_type = other DNA
tccaggtcactgttaggaagacatc

SEQ ID NO: 7 - DNA, 26 bp, synthetic construct
  misc_feature 1..26: note = Synthetic primer: CVL20
  source 1..26: mol_type = other DNA
gctcgattacctagattgaccgagac

SEQ ID NO: 8 - DNA, 23 bp, synthetic construct
  misc_feature 1..23: note = Synthetic primer: CVL17
  source 1..23: mol_type = other DNA
caagatgcaccttctctccagtg

SEQ ID NO: 9 - DNA, 24 bp, synthetic construct
  misc_feature 1..24: note = Synthetic primer: CVL154
  source 1..24: mol_type = other DNA
atcacactcgagctgacatctgtc

SEQ ID NO: 10 - DNA, 22 bp, synthetic construct
  misc_feature 1..22: note = Synthetic primer: CVL152
  source 1..22: mol_type = other DNA
cgtgagtacctacatgctgaca

SEQ ID NO: 11 - DNA, 23 bp, synthetic construct
  misc_feature 1..23: note = Synthetic primer: CVL25
  source 1..23: mol_type = other DNA
cggattgatacgctgagttgctg

SEQ ID NO: 12 - DNA, 23 bp, synthetic construct
  misc_feature 1..23: note = Synthetic primer: CVL19
  source 1..23: mol_type = other DNA
taactctgcagtcgctctacctc

SEQ ID NO: 13 - DNA, 22 bp, synthetic construct
  misc_feature 1..22: note = Synthetic primer: CVL27
  source 1..22: mol_type = other DNA
cagtctgagtcatgccatgctg

SEQ ID NO: 14 - DNA, 26 bp, synthetic construct
  misc_feature 1..26: note = Synthetic primer: CVL26
  source 1..26: mol_type = other DNA
gcattcagaatctcagctatggactg

SEQ ID NO: 15 - DNA, 24 bp, synthetic construct
  misc_feature 1..24: note = Synthetic primer: CVL33
  source 1..24: mol_type = other DNA
tcaatgatgatgtgagctacacgc

SEQ ID NO: 16 - DNA, 25 bp, synthetic construct
  misc_feature 1..25: note = Synthetic primer: CVL32
  source 1..25: mol_type = other DNA
gctctgatggaactcagtacgaatc

SEQ ID NO: 17 - DNA, 26 bp, synthetic construct
  misc_feature 1..26: note = Synthetic primer: CVL37
  source 1..26: mol_type = other DNA
ggttctctattgaactcaaccctgag

SEQ ID NO: 18 - DNA, 25 bp, synthetic construct
  misc_feature 1..25: note = Synthetic primer: CVL36
  source 1..25: mol_type = other DNA
gtgaccgatccatgaatctcattgc

SEQ ID NO: 19 - DNA, 26 bp, synthetic construct
  misc_feature 1..26: note = Synthetic primer: CVL42
  source 1..26: mol_type = other DNA
accaaggggaaaaccaagattaatcc